(19) United States
(12) Patent Application Publication
(10) Pub. No.: US 2025/0382384 A1
(43) Pub. Date: Dec. 18, 2025

(54) MUC1 Antibodies and Methods of Use

(71) Applicant: BeOne Medicines I GmbH, Basel (CH)

(72) Inventors: Qi Liu, Beijing (CN); Xin Chen, Beijing (CN); Yun Chen, Shanghai (CN); Liu Xue, Beijing (CN); Renke Li, Shanghai (CN); Peiqi Li, Shanghai (CN); Ting Shao, Shanghai (CN); Wenjie Wang, Beijing (CN); Jie Pan, Shanghai (CN); Zhuo Li, Shanghai (CN); Xiaoyan Tang, Beijing (CN); Chichi Huang, Cambridge, MA (US)

(73) Assignee: BeOne Medicines I GmbH, Basel (CH)

(21) Appl. No.: 19/316,085

(22) Filed: Sep. 2, 2025

Related U.S. Application Data
(63) Continuation of application No. PCT/CN2024/079596, filed on Mar. 1, 2024.

(30) Foreign Application Priority Data
Mar. 3, 2023 (WO) PCT/CN2023/079507
Mar. 12, 2023 (WO) PCT/CN2023/080961
Jul. 17, 2023 (WO) PCT/CN2023/107724

(51) Int. Cl. C07K 16/30

(52) U.S. Cl. CPC C07K 16/3092; C07K 2317/24; C07K 2317/34; C07K 2317/622; C07K 2317/77; C07K 2317/92; C07K 2317/94

(57) ABSTRACT

The present disclosure provides for antibodies and antigen-binding fragments thereof that bind to human MUC1 and pharmaceutical compositions comprising said antibodies.

### CROSS-REFERENCE TO RELATED APPLICATION

This application is a continuation of International Application No. PCT/CN2024/079596, filed Mar. 1, 2024, entitled “MUC1 Antibodies and Methods of Use”, which claims the benefit of priority of PCT Application No. PCT Application No. PCT/CN2023/079507, filed Mar. 3, 2023 entitled “MUC1 Antibodies and Methods of Use,” PCT/CN2023/080961, filed Mar. 12, 2023, entitled “MUC1 Antibodies and Methods of Use,” and PCT Application No. PCT/CN2023/107724, filed Jul. 17, 2023, entitled “MUC1 and CD16A Antibodies and Methods of Use,” which are hereby incorporated by reference in their entireties.

### SEQUENCE LISTING

The present application is filed with a Sequence Listing in electronic format. The Sequence Listing is provided as a file entitled “01368-0023-00PCT_SL.xml” created on Feb. 27, 2024, which is 121,339 bytes in size. The information in the electronic format of the sequence listing is incorporated herein by reference in its entirety.

### FIELD OF THE DISCLOSURE

Disclosed herein are antibodies or antigen-binding fragments thereof that bind to human MUC1 and methods of use thereof.

### BACKGROUND

Mucin 1 (MUC1; also known as CA15-3, EMA, MCD, PEM, PUM, KL-6, MAM6, MCKD, PEMT, CD227, H23AG, MCKD1, ADMCKD, ADTKD2) is a single-pass transmembrane glycoprotein belonging to the mucin family. MUC1 is heavily glycosylated and is expressed on the apical surface of epithelial cells that line the mucosal surfaces of various normal tissues including lung, mammary gland, stomach, pancreas, and uterus. MUC1 plays an essential role in forming protective mucous barriers on these epithelial surfaces.

MUC1 is translated as a single polypeptide, whose extracellular portion undergoes autocleavage into two subunits at the SEA (sea-urchin sperm protein, enterokinase and agrin) domain. The cleaved extracellular membrane-distal subunit, containing 20-125 repeats of a 20-amino acid sequence (variable number tandem repeat, VNTR), forms a heterodimeric complex with the membrane-proximal subunit via a strong non-covalent interaction; the membrane-proximal subunit consists of a 58-amino acid (aa) extracellular domain, a 28-aa transmembrane domain, and a 72-aa cytoplasmic tail which intersects with multiple oncogenic signaling pathways.

MUC1 is aberrantly overexpressed in a variety of high-incidence human cancers, including lung, breast, colon, gastric, esophageal, and ovarian cancers. In addition to upregulated expression, MUC1 hypo-glycosylation and changes in cellular localization have also been associated with cancers. Moreover, the cleaved membrane-distal subunit has a tendency to be shed from the surface of cancer cells in association with increased plasma levels.

Considering its overexpression in diverse human carcinomas, MUC1 is an attractive tumor associated antigen. However, previous attempts targeting the membrane-distal (MUC1 N-terminal) subunit, such as AS1402 (huHMFG-1) and BrevaRex (AR-20.5), have been unsuccessful. This is in part due to the circulating pools of shed MUC1 that preclude targeting of antibodies to the surface of MUC1-expressing tumor cells. In contrast, the non-shed MUC1 membrane-proximal (MUC1 C-terminal) subunit, which functions as an oncoprotein, represents an attractive target for the development of antibody-based therapeutics.

The current disclosure provides for anti-MUC1 antibodies that target the MUC1 membrane-proximal region (specifically the MUC1-SEA domain, which is adjacent to the transmembrane region at the MUC1-C terminal) with minimal interference by shed MUC1.

### SUMMARY OF THE DISCLOSURE

The present disclosure is directed to anti-MUC1 antibodies and antigen-binding fragments thereof.

In embodiments, the present disclosure is directed to anti-MUC1 antibodies and antigen-binding fragments thereof, which specifically bind to cancer cells and do not bind to normal cells.

In embodiments, the present disclosure is directed to an anti-MUC1 antibody or antigen-binding fragment thereof, comprising an antibody or binding fragment thereof that specifically binds to the SEA domain of human MUC1 (SEQ ID NO: 1) at amino acids 1036 to 1155 (SEQ ID NO: 2).

In embodiments, the present disclosure is directed to an anti-MUC1 antibody or antigen-binding fragment that specifically binds to human MUC1, comprising:
(i). a heavy chain variable region that comprises (a) a HCDR1 (Heavy Chain Complementarity Determining Region 1) of SEQ ID NO: 4, (b) a HCDR2 of SEQ ID NO: 5, and (c) a HCDR3 of SEQ ID NO: 6, and a light chain variable region that comprises: (d) a LCDR1 (Light Chain Complementarity Determining Region 1) of SEQ ID NO: 7, (e) a LCDR2 of SEQ ID NO: 8, and (f) a LCDR3 of SEQ ID NO: 9;
(ii). a heavy chain variable region that comprises (a) a HCDR1 of SEQ ID NO: 4, (b) a HCDR2 of SEQ ID NO: 5, and (c) a HCDR3 of SEQ ID NO: 6, and a light chain variable region that comprises: (d) a LCDR1 of SEQ ID NO: 7, (e) a LCDR2 of SEQ ID NO: 56, and (f) a LCDR3 of SEQ ID NO: 9;
(iii). a heavy chain variable region that comprises (a) a HCDR1 of SEQ ID NO: 14, (b) a HCDR2 of SEQ ID NO: 15, and (c) a HCDR3 of SEQ ID NO: 16, and a light chain variable region that comprises: (d) a LCDR1 of SEQ ID NO: 17, (e) a LCDR2 of SEQ ID NO: 18, and (f) a LCDR3 of SEQ ID NO: 19;
(iv). a heavy chain variable region that comprises (a) a HCDR1 of SEQ ID NO: 78, (b) a HCDR2 of SEQ ID NO: 79, and (c) a HCDR3 of SEQ ID NO: 16, and a light chain variable region that comprises: (d) a LCDR1 of SEQ ID NO: 17, (e) a LCDR2 of SEQ ID NO: 18, and (f) a LCDR3 of SEQ ID NO: 19;
(v). a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (e) a LCDR2 of SEQ ID NO: 28, and (f) a LCDR3 of SEQ ID NO: 29;
(vi). a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (e) a LCDR2 of SEQ ID NO: 65, and (f) a LCDR3 of SEQ ID NO: 29; or
(vii). a heavy chain variable region that comprises (a) a HCDR1 of SEQ ID NO: 24, (b) a HCDR2 of SEQ ID NO: 74, and (c) a HCDR3 of SEQ ID NO: 26, and a light chain variable region that comprises: (d) a LCDR1 of SEQ ID NO: 27, (e) a LCDR2 of SEQ ID NO: 65, and (f) a LCDR3 of SEQ ID NO: 29.

In embodiments, the anti-MUC1 antibody or antigen-binding fragment thereof, comprises:
(i). a heavy chain variable region (VH) comprising an amino acid sequence at least 90, 91, 92, 93, 94, 95, 96, 97, 98, or 99% identical to SEQ ID NO: 10, and a light chain variable region (VL) comprising an amino acid sequence at least 90, 91, 92, 93, 94, 95, 96, 97, 98, or 99% identical to SEQ ID NO: 11;
(ii). a heavy chain variable region (VH) comprising an amino acid sequence at least 90, 91, 92, 93, 94, 95, 96, 97, 98, or 99% identical to SEQ ID NO: 20, and a light chain variable region (VL) comprising an amino acid sequence at least 90, 91, 92, 93, 94, 95, 96, 97, 98, or 99% identical to SEQ ID NO: 21;
(iii). a heavy chain variable region (VH) comprising an amino acid sequence at least 90, 91, 92, 93, 94, 95, 96, 97, 98, or 99% identical to SEQ ID NO: 30, and a light chain variable region (VL) comprising an amino acid sequence at least 90, 91, 92, 93, 94, 95, 96, 97, 98, or 99% identical to SEQ ID NO: 31;
(iv). a heavy chain variable region (VH) comprising an amino acid sequence at least 90, 91, 92, 93, 94, 95, 96, 97, 98, or 99% identical to SEQ ID NO: 57, and a light chain variable region (VL) comprising an amino acid sequence at least 90, 91, 92, 93, 94, 95, 96, 97, 98, or 99% identical to SEQ ID NO: 59;
(v). a heavy chain variable region (VH) comprising an amino acid sequence at least 90, 91, 92, 93, 94, 95, 96, 97, 98, or 99% identical to SEQ ID NO: 57, and a light chain variable region (VL) comprising an amino acid sequence at least 90, 91, 92, 93, 94, 95, 96, 97, 98, or 99% identical to SEQ ID NO: 60;
(vi). a heavy chain variable region (VH) comprising an amino acid sequence at least 90, 91, 92, 93, 94, 95, 96, 97, 98, or 99% identical to SEQ ID NO: 58, and a light chain variable region (VL) comprising an amino acid sequence at least 90, 91, 92, 93, 94, 95, 96, 97, 98, or 99% identical to SEQ ID NO: 59;
(vii). a heavy chain variable region (VH) comprising an amino acid sequence at least 90, 91, 92, 93, 94, 95, 96, 97, 98, or 99% identical to SEQ ID NO: 58, and a light chain variable region (VL) comprising an amino acid sequence at least 90, 91, 92, 93, 94, 95, 96, 97, 98, or 99% identical to SEQ ID NO: 60;
(viii). a heavy chain variable region (VH) comprising an amino acid sequence at least 90, 91, 92, 93, 94, 95, 96, 97, 98, or 99% identical to SEQ ID NO: 61, and a light chain variable region (VL) comprising an amino acid sequence at least 90, 91, 92, 93, 94, 95, 96, 97, 98, or 99% identical to SEQ ID NO: 62;
(ix). a heavy chain variable region (VH) comprising an amino acid sequence at least 90, 91, 92, 93, 94, 95, 96, 97, 98, or 99% identical to SEQ ID NO: 61, and a light chain variable region (VL) comprising an amino acid sequence at least 90, 91, 92, 93, 94, 95, 96, 97, 98, or 99% identical to SEQ ID NO: 66;
(x). a heavy chain variable region (VH) comprising an amino acid sequence at least 90, 91, 92, 93, 94, 95, 96, 97, 98, or 99% identical to SEQ ID NO: 61, and a light chain variable region (VL) comprising an amino acid sequence at least 90, 91, 92, 93, 94, 95, 96, 97, 98, or 99% identical to SEQ ID NO: 68;
(xi). a heavy chain variable region (VH) comprising an amino acid sequence at least 90, 91, 92, 93, 94, 95, 96, 97, 98, or 99% identical to SEQ ID NO: 71, and a light chain variable region (VL) comprising an amino acid sequence at least 90, 91, 92, 93, 94, 95, 96, 97, 98, or 99% identical to SEQ ID NO: 72;
(xii). a heavy chain variable region (VH) comprising an amino acid sequence at least 90, 91, 92, 93, 94, 95, 96, 97, 98, or 99% identical to SEQ ID NO: 75, and a light chain variable region (VL) comprising an amino acid sequence at least 90, 91, 92, 93, 94, 95, 96, 97, 98, or 99% identical to SEQ ID NO: 76; or
(xiii). a heavy chain variable region (VH) comprising an amino acid sequence at least 90, 91, 92, 93, 94, 95, 96, 97, 98, or 99% identical to SEQ ID NO: 80, and a light chain variable region (VL) comprising an amino acid sequence at least 90, 91, 92, 93, 94, 95, 96, 97, 98, or 99% identical to SEQ ID NO: 81.

In embodiments, the anti-MUC1 antibody or antigen-binding fragment thereof comprises:
(i). a heavy chain variable region (VH) that comprises SEQ ID NO: 10, and a light chain variable region (VL) that comprises SEQ ID NO: 11;
(ii). a heavy chain variable region (VH) that comprises SEQ ID NO: 20, and a light chain variable region (VL) that comprises SEQ ID NO: 21;
(iii). a heavy chain variable region (VH) that comprises SEQ ID NO: 30, and a light chain variable region (VL) that comprises SEQ ID NO: 31;
(iv). a heavy chain variable region (VH) that comprises SEQ ID NO: 57, and a light chain variable region (VL) that comprises SEQ ID NO: 59;
(v). a heavy chain variable region (VH) that comprises SEQ ID NO: 57, and a light chain variable region (VL) that comprises SEQ ID NO: 60;
(vi). a heavy chain variable region (VH) that comprises SEQ ID NO: 58, and a light chain variable region (VL) that comprises SEQ ID NO: 59;
(vii). a heavy chain variable region (VH) that comprises SEQ ID NO: 58, and a light chain variable region (VL) that comprises SEQ ID NO: 60;
(viii). a heavy chain variable region (VH) that comprises SEQ ID NO: 61, and a light chain variable region (VL) that comprises SEQ ID NO: 62;
(ix). a heavy chain variable region (VH) that comprises SEQ ID NO: 61, and a light chain variable region (VL) that comprises SEQ ID NO: 66;
(x). a heavy chain variable region (VH) that comprises SEQ ID NO: 61, and a light chain variable region (VL) that comprises SEQ ID NO: 68;
(xi). a heavy chain variable region (VH) that comprises SEQ ID NO: 71, and a light chain variable region (VL) that comprises SEQ ID NO: 72;
(xii). a heavy chain variable region (VH) that comprises SEQ ID NO: 75, and a light chain variable region (VL) that comprises SEQ ID NO: 76; or
(xiii). a heavy chain variable region (VH) that comprises SEQ ID NO: 80, and a light chain variable region (VL) that comprises SEQ ID NO: 81.

In embodiments, in the anti-MUC1 antibody or antigen-binding fragment thereof, one, two, three, four, five, six, seven, eight, nine, or ten amino acids within SEQ ID NO: 10, 11, 20, 21, 30, 31, 57, 58, 59, 60, 61, 62, 66, 68, 71, 72, 75, 76, 80, or 81 have been inserted, deleted, or substituted.

In embodiments, the anti-MUC1 antibody or antigen-binding fragment thereof is a monoclonal antibody, a chimeric antibody, a humanized antibody, a human engineered antibody, a single chain antibody (scFv), a Fab fragment, a Fab′ fragment, or a F(ab′)₂ fragment.

In embodiments, the antibody or antigen-binding fragment thereof comprises a scFv comprising a heavy chain variable region (VH) comprising an amino acid sequence at least 90, 91, 92, 93, 94, 95, 96, 97, 98, or 99% identical to the amino acid of SEQ ID NO: 57, SEQ ID NO: 71, SEQ ID NO: 75, or SEQ ID NO: 80, and a VL comprising an amino acid sequence at least 90, 91, 92, 93, 94, 95, 96, 97, 98, or 99% identical to SEQ ID NO: 60, SEQ ID NO: 72, SEQ ID NO: 76, or SEQ ID NO: 81.

In embodiments, the antibody or antigen-binding fragment thereof comprises a scFv comprising a VH having the amino acid sequence of SEQ ID NO: 57, SEQ ID NO: 71, SEQ ID NO: 75, or SEQ ID NO: 80, and a VL having an amino acid of SEQ ID NO: 60, SEQ ID NO: 72, SEQ ID NO: 76, or SEQ ID NO: 81.

In embodiments, the antibody or antigen-binding fragment thereof comprises a scFv comprising:
(i). a VH comprising an amino acid sequence at least 90, 91, 92, 93, 94, 95, 96, 97, 98, or 99% identical to SEQ ID NO: 57, and a VL comprising an amino acid sequence at least 90, 91, 92, 93, 94, 95, 96, 97, 98, or 99% identical to SEQ ID NO: 60;
(ii). a VH comprising an amino acid sequence at least 90, 91, 92, 93, 94, 95, 96, 97, 98, or 99% identical to SEQ ID NO: 71, and a VL comprising an amino acid sequence at least 90, 91, 92, 93, 94, 95, 96, 97, 98, or 99% identical to SEQ ID NO: 72;
(iii). a VH comprising an amino acid sequence at least 90, 91, 92, 93, 94, 95, 96, 97, 98, or 99% identical to SEQ ID NO: 75, and a VL comprising an amino acid sequence at least 90, 91, 92, 93, 94, 95, 96, 97, 98, or 99% identical to SEQ ID NO: 76; or
(iv). a VH comprising an amino acid sequence at least 90, 91, 92, 93, 94, 95, 96, 97, 98, or 99% identical to SEQ ID NO: 80, and a VL comprising an amino acid sequence at least 90, 91, 92, 93, 94, 95, 96, 97, 98, or 99% identical to SEQ ID NO: 81.

In embodiments, the antibody or antigen-binding fragment thereof comprises a scFv comprising:
(i). a VH having the amino acid sequence of SEQ ID NO: 57, and a VL having the amino acid sequence of SEQ ID NO: 60;
(ii). a VH having the amino acid sequence of SEQ ID NO: 71, and a VL having the amino acid sequence of SEQ ID NO: 72;
(iii). a VH having the amino acid sequence of SEQ ID NO: 75, and a VL having the amino acid sequence of SEQ ID NO: 76; or
(iv). a VH having the amino acid sequence of SEQ ID NO: 80, and a VL having the amino acid sequence of SEQ ID NO: 81.

In embodiments, in the anti-MUC1 antibody or antigen-binding fragment thereof, one, two, three, four, five, six, seven, eight, nine, or ten amino acids within SEQ ID NO: 57, 60, 71, 72, 75, 76, 80, or 81 have been inserted, deleted, or substituted.

In embodiments, the VH and VL of a scFv of the anti-MUC1 antibody or antigen-binding fragment thereof are connected via an amino acid linker. The amino acid linker may have the amino acid sequence of SEQ ID NO: 83 or 84.

In embodiments, the antibody or antigen-binding fragment thereof comprises a scFv having the amino acid sequence of SEQ ID NO: 70, SEQ ID NO: 73, SEQ ID NO: 77, or SEQ ID NO: 82.

In embodiments, the antibody or antigen-binding fragment thereof comprises a scFv having an amino acid sequence at least 90, 91, 92, 93, 94, 95, 96, 97, 98, or 99% identical to SEQ ID NO: 70, 73, 77, or 82.

In embodiments, in the anti-MUC1 antibody or antigen-binding fragment thereof, one, two, three, four, five, six, seven, eight, nine, or ten amino acids within SEQ ID NO: 70, 73, 77, or 82 have been inserted, deleted, or substituted.

In embodiments, the anti-MUC1 antibody or antigen-binding fragment thereof exhibit antibody dependent cellular cytotoxicity (ADCC) or complement dependent cytotoxicity (CDC). In embodiments, the ADCC is against MUC1 expressing target cells.

In embodiments, the anti-MUC1 antibody or antigen-binding fragment thereof has reduced glycosylation or no glycosylation or is hypofucosylated.

In embodiments, the anti-MUC1 antibody or antigen-binding fragment thereof comprises increased bisecting GlcNac structures.

In embodiments, the anti-MUC1 antibody or antigen-binding fragment thereof is of IgG1, IgG2, IgG3, or IgG4 isotype. In embodiments, the antibody or antigen-binding fragment thereof comprises a heavy chain constant region of the subclass of IgG1, IgG2, IgG3, or IgG4, and/or a light chain constant region of the type of kappa or lambda. In one embodiment, the antibody or antigen-binding fragment thereof comprises an Fc domain of wild-type human IgG1 (also referred as human IgG1wt or huIgG1) or IgG2. In one embodiment, antibody or antigen-binding fragment thereof comprises an IgG1 Fc domain.

In embodiments, the present disclosure is directed to a pharmaceutical composition comprising the anti-MUC1 antibody or antigen-binding fragment thereof and a pharmaceutically acceptable carrier.

In embodiments, the present disclosure is directed to an isolated nucleic acid that encodes the anti-MUC1 antibody or antigen-binding fragment thereof disclosed herein.

In embodiments, the present disclosure is directed to a vector comprising the nucleic acid disclosed herein.

In embodiments, the present disclosure is directed to a host cell comprising the nucleic acid disclosed herein or the vector disclosed herein.

In embodiments, the present disclosure is directed to a process for producing an anti-MUC1 antibody or antigen-binding fragment thereof comprising cultivating the host cell disclosed herein and recovering the antibody or antigen-binding fragment from the culture.

In one embodiment, the anti-MUC1 antibody or an antigen-binding fragment thereof comprises one or more complementarity determining regions (CDRs) comprising an amino acid sequence selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 56, SEQ ID NO: 14, SEQ ID NO: 15, SEQ ID NO: 16, SEQ ID NO: 17, SEQ ID NO: 18, SEQ ID NO: 19, SEQ ID NO: 78, SEQ ID NO: 79, SEQ ID NO: 24, SEQ ID NO: 25, SEQ ID NO: 26, SEQ ID NO: 27, SEQ ID NO: 28, SEQ ID NO: 29, and SEQ ID NO: 65.

In another embodiment, the anti-MUC1 antibody or an antigen-binding fragment thereof comprises: (a) a heavy chain variable region comprising one or more complementarity determining regions (HCDRs) comprising an amino acid sequence selected from the group consisting of SEQ ID NO: 4, SEQ ID NO: 5, SEQ ID NO: 6, SEQ ID NO: 14, SEQ ID NO: 15, SEQ ID NO: 16, SEQ ID NO: 24, SEQ ID NO: 25, SEQ ID NO: 26, SEQ ID NO: 74, SEQ ID NO: 78, or SEQ ID NO: 79; and/or (b) a light chain variable region comprising one or more complementarity determining regions (LCDRs) having an amino acid sequence selected from the group consisting of SEQ ID NO: 7, SEQ ID NO: 8, SEQ ID NO: 9, SEQ ID NO: 56, SEQ ID NO: 17, SEQ ID NO: 18, SEQ ID NO: 19, SEQ ID NO: 27, SEQ ID NO: 28, SEQ ID NO: 29, and SEQ ID NO: 65.

In another embodiment, the anti-MUC1 antibody or an antigen-binding fragment thereof comprises: (a) a heavy chain variable region comprising three complementarity determining regions (HCDRs) which are HCDR1 comprising an amino acid sequence of SEQ ID NO: 4; SEQ ID NO: 14, SEQ ID NO: 24, or SEQ ID NO: 78; HCDR2 comprising an amino acid sequence of SEQ ID NO: 5, SEQ ID NO: 15, SEQ ID NO: 25, SEQ ID NO: 74, or SEQ ID NO: 79; and HCDR3 comprising an amino acid sequence of SEQ ID NO: 6, SEQ ID NO: 16, or SEQ ID NO: 26, and/or (b) a light chain variable region comprising three complementarity determining regions (LCDRs) which are LCDR1 comprising an amino acid sequence of SEQ ID NO: 7, SEQ ID NO: 17, or SEQ ID NO: 27; LCDR2 comprising an amino acid sequence of SEQ ID NO: 8, SEQ ID NO: 18, SEQ ID NO: 28, SEQ ID NO: 56, or SEQ ID NO: 65; and LCDR3 comprising an amino acid sequence of SEQ ID NO: 9, SEQ ID NO: 19, or SEQ ID NO: 29.

In another embodiment, the anti-MUC1 antibody or an antigen-binding fragment thereof comprises:
(a) a heavy chain variable region comprising three complementarity determining regions (HCDRs) which are
HCDR1 comprising the amino acid sequence of SEQ ID NO: 4,
HCDR2 comprising the amino acid sequence of SEQ ID NO: 5, and
HCDR3 comprising the amino acid sequence of SEQ ID NO: 6; or
HCDR1 comprising the amino acid sequence of SEQ ID NO: 14,
HCDR2 comprising the amino acid sequence of SEQ ID NO: 15, and
HCDR3 comprising the amino acid sequence of SEQ ID NO: 16; or
HCDR1 comprising the amino acid sequence of SEQ ID NO: 24,
HCDR2 comprising the amino acid sequence of SEQ ID NO: 25, and
HCDR3 comprising the amino acid sequence of SEQ ID NO: 26; or
HCDR1 comprising the amino acid sequence of SEQ ID NO: 24,
HCDR2 comprising the amino acid sequence of SEQ ID NO: 74, and
HCDR3 comprising the amino acid sequence of SEQ ID NO: 26; or
HCDR1 comprising the amino acid sequence of SEQ ID NO: 78,
HCDR2 comprising the amino acid sequence of SEQ ID NO: 79, and
HCDR3 comprising the amino acid sequence of SEQ ID NO: 16; and/or
(b) a light chain variable region comprising three complementarity determining regions (LCDRs) which are
LCDR1 comprising the amino acid sequence of SEQ ID NO: 7,
LCDR2 comprising the amino acid sequence of SEQ ID NO: 8, and
LCDR3 comprising the amino acid sequence of SEQ ID NO: 9; or
LCDR1 comprising the amino acid sequence of SEQ ID NO: 17,
LCDR2 comprising the amino acid sequence of SEQ ID NO: 18, and
LCDR3 comprising the amino acid sequence of SEQ ID NO: 19; or
LCDR1 comprising the amino acid sequence of SEQ ID NO: 27,
LCDR2 comprising the amino acid sequence of SEQ ID NO: 28, and
LCDR3 comprising the amino acid sequence of SEQ ID NO: 29; or
LCDR1 comprising the amino acid sequence of SEQ ID NO: 7,
LCDR2 comprising the amino acid sequence of SEQ ID NO: 56, and
LCDR3 comprising the amino acid sequence of SEQ ID NO: 9; or
LCDR1 comprising the amino acid sequence of SEQ ID NO: 27,
LCDR2 comprising the amino acid sequence of SEQ ID NO: 65, and
LCDR3 comprising the amino acid sequence of SEQ ID NO: 29.

In another embodiment, the anti-MUC1 antibody or the antigen-binding fragment thereof comprises: a heavy chain variable region that comprises (a) a HCDR1 of SEQ ID NO: 4, (b) a HCDR2 of SEQ ID NO: 5, and (c) a HCDR3 of SEQ ID NO: 6, and a light chain variable region that comprises: (d) a LCDR1 of SEQ ID NO: 7, (e) a LCDR2 of SEQ ID NO: 8, and (f) a LCDR3 of SEQ ID NO: 9.

In another embodiment, the anti-MUC1 antibody or the antigen-binding fragment thereof comprises: a heavy chain variable region that comprises (a) a HCDR1 of SEQ ID NO: 4, (b) a HCDR2 of SEQ ID NO: 5, and (c) a HCDR3 of SEQ ID NO: 6, and a light chain variable region that comprises: (d) a LCDR1 of SEQ ID NO: 7, (e) a LCDR2 of SEQ ID NO: 56, and (f) a LCDR3 of SEQ ID NO: 9.

In another embodiment, the anti-MUC1 antibody or the antigen-binding fragment thereof comprises: a heavy chain variable region that comprises (a) a HCDR1 of SEQ ID NO: 14, (b) a HCDR2 of SEQ ID NO: 15, and (c) a HCDR3 of SEQ ID NO: 16, and a light chain variable region that comprises: (d) a LCDR1 of SEQ ID NO: 17, (e) a LCDR2 of SEQ ID NO: 18, and (f) a LCDR3 of SEQ ID NO: 19.

In another embodiment, the anti-MUC1 antibody or the antigen-binding fragment thereof comprises: a heavy chain variable region that comprises (a) a HCDR1 of SEQ ID NO: 78, (b) a HCDR2 of SEQ ID NO: 79, and (c) a HCDR3 of SEQ ID NO: 16, and a light chain variable region that comprises: (d) a LCDR1 of SEQ ID NO: 17, (e) a LCDR2 of SEQ ID NO: 18, and (f) a LCDR3 of SEQ ID NO: 19.

In another embodiment, the anti-MUC1 antibody or the antigen-binding fragment thereof comprises: a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (e) a LCDR2 of SEQ ID NO: 28, and (f) a LCDR3 of SEQ ID NO: 29.

In another embodiment, the anti-MUC1 antibody or the antigen-binding fragment thereof comprises: a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (e) a LCDR2 of SEQ ID NO: 65, and (f) a LCDR3 of SEQ ID NO: 29.

In another embodiment, the anti-MUC1 antibody or the antigen-binding fragment thereof comprises: a heavy chain variable region that comprises (a) a HCDR1 of SEQ ID NO: 24, (b) a HCDR2 of SEQ ID NO: 74, and (c) a HCDR3 of SEQ ID NO: 26, and a light chain variable region that comprises: (d) a LCDR1 of SEQ ID NO: 27, (e) a LCDR2 of SEQ ID NO: 65, and (f) a LCDR3 of SEQ ID NO: 29.

In one embodiment, the anti-MUC1 antibody or antigen-binding fragment thereof comprises: (a) a heavy chain variable region having an amino acid sequence of SEQ ID NO: 10, SEQ ID NO: 20, SEQ ID NO: 30, SEQ ID NO: 57, SEQ ID NO: 58, SEQ ID NO: 61, SEQ ID NO: 71, SEQ ID NO: 75, or SEQ ID NO: 80, or an amino acid sequence at least 95%, 96%, 97%, 98%, or 99% identical to any one of SEQ ID NO: 10, SEQ ID NO: 20, SEQ ID NO: 30, SEQ ID NO: 57, SEQ ID NO: 58, SEQ ID NO: 61, SEQ ID NO: 71, SEQ ID NO: 75, or SEQ ID NO: 80, and/or (b) a light chain variable region comprising an amino acid sequence of SEQ ID NO: 11, SEQ ID NO: 21, SEQ ID NO: 31, SEQ ID NO: 59, SEQ ID NO: 60, SEQ ID NO: 62, SEQ ID NO: 66, SEQ ID NO: 68, SEQ ID NO: 72, SEQ ID NO: 76, or SEQ ID NO: 81, or an amino acid sequence at least 95%, 96%, 97%, 98%, or 99% identical to any one of SEQ ID NO: 11, SEQ ID NO: 21, SEQ ID NO: 31, SEQ ID NO: 59, SEQ ID NO: 60, SEQ ID NO: 62, SEQ ID NO: 66, SEQ ID NO: 68, SEQ ID NO: 72, SEQ ID NO: 76, or SEQ ID NO: 81.

In another embodiment, the anti-MUC1 antibody or an antigen-binding fragment thereof comprises: (a) a heavy chain variable region comprising an amino acid sequence of SEQ ID NO: 10, SEQ ID NO: 20, SEQ ID NO: 30, SEQ ID NO: 57, SEQ ID NO: 58, SEQ ID NO: 61, SEQ ID NO: 71, SEQ ID NO: 75, or SEQ ID NO: 80, or an amino acid sequence comprising one, two, or three amino acid substitutions in the amino acid sequence of SEQ ID NO: 10, SEQ ID NO: 20, SEQ ID NO: 30, SEQ ID NO: 57, SEQ ID NO: 58, SEQ ID NO: 61, SEQ ID NO: 71, SEQ ID NO: 75, or SEQ ID NO: 80, and/or (b) a light chain variable region comprising an amino acid sequence of SEQ ID NO: 11, SEQ ID NO: 21, SEQ ID NO: 31, SEQ ID NO: 59, SEQ ID NO: 60, SEQ ID NO: 62, SEQ ID NO: 66, SEQ ID NO: 68, SEQ ID NO: 72, SEQ ID NO: 76, or SEQ ID NO: 81, or an amino acid sequence comprising one, two, three, four, or five amino acid substitutions in the amino acid of SEQ ID NO: 11, SEQ ID NO: 21, SEQ ID NO: 31, SEQ ID NO: 59, SEQ ID NO: 60, SEQ ID NO: 62, SEQ ID NO: 66, SEQ ID NO: 68, SEQ ID NO: 72, SEQ ID NO: 76, or SEQ ID NO: 81. In another embodiment, the amino acid substitutions are conservative amino acid substitutions.

In one embodiment, the anti-MUC1 antibody binds to MUC1 with a binding affinity (K_{D}) of from 1×10⁻⁶ M to 1×10⁻¹⁰ M, or to 1×10⁻¹¹ M. In another embodiment, the anti-MUC1 antibody binds to MUC1 with a K_{D} of about 1×10⁻⁶ M, about 1×10⁻⁷ M, about 1×10⁻⁸ M, about 1×10⁻⁹ M, about 1×10⁻¹⁰ M, or about 1×10⁻¹¹ M.

In another embodiment, the anti-human MUC1 antibody or antigen-binding fragment thereof shows a cross-species binding activity to cynomolgus MUC1.

In some embodiments, the present disclosure relates to isolated nucleic acids comprising nucleotide sequences encoding the amino acid sequence of the anti-MUC1 antibody or antigen-binding fragment. In one embodiment, the isolated nucleic acid comprises a VH nucleotide sequence of SEQ ID NO: 12, SEQ ID NO: 22, SEQ ID NO: 32, or SEQ ID NO: 63, or a nucleotide sequence comprising at least 95%, 96%, 97%, 98%, or 99% identity to SEQ ID NO: 12, SEQ ID NO: 22, SEQ ID NO: 32, or SEQ ID NO: 63, and encodes the VH region of the antibody or an antigen-binding fragment of the present disclosure. In one embodiment, the isolated nucleic acid comprises a VL nucleotide sequence of SEQ ID NO: 13, SEQ ID NO: 23, SEQ ID NO: 33, SEQ ID NO: 64, SEQ ID NO: 67, or SEQ ID NO: 69, or a nucleotide sequence comprising at least 95%, 96%, 97%, 98%, or 99% identity to SEQ ID NO: 13, SEQ ID NO: 23, SEQ ID NO: 33, SEQ ID NO: 64, SEQ ID NO: 67, or SEQ ID NO: 69, and encodes the VL region of the antibody or an antigen-binding fragment of the present disclosure.

In some embodiments, the present disclosure provides anti-human MUC1 antibodies or antigen-binding fragments thereof the exhibit specific binding to and high affinity for human MUC1.

### BRIEF DESCRIPTION OF THE DRAWINGS

FIG. 1 is a schematic diagram of MUC1-SEA-mIgG2a (top) and MUC1-SEA-huIgG1 (bottom), in which “N” is the N-terminus and “C” is the C-terminus.

FIGS. 2A-2F show the binding affinity of purified MUC1 antibodies with human and cyno MUC1 overexpression cells by FACS assay with human IgG1 as a negative control. FIGS. 2A and 2B show chimeric anti-MUC1 monoclonal antibody BG138P binding affinity with human and cyno cells that overexpress MUC1. FIGS. 2C and 2D show chimeric BG346P binding affinity with human and cyno cells that overexpress MUC1. FIGS. 2E and 2F show chimeric BG219P binding affinity with human and cyno cells that overexpress MUC1.

FIGS. 3A-3C show epitope binning determination by competitive SPR assays, in which purified human MUC1-mFc antigen was flowed over a chip surface and captured by anti-mouse IgG antibody.

FIGS. 4A-4F show the effect of soluble MUC1 on MUC1 antibodies binding to MUC1 expressing cells. FIGS. 4A-C show the binding profiles of chBG138P and chBG219P at different concentrations (30, 3, and 0.3 μg/ml, respectively) in the presence of soluble MUC1, with HMFG1 as a positive control, and mIgG and hIgG1 as negative controls. FIGS. 4D-F show the binding profiles of chBG138P and chBG346P at different concentrations (30, 3, and 0.3 μg/ml, respectively) in the presence of soluble MUC1, with HMFG1 as a positive control, and mIgG and hIgG1 as negative controls.

FIGS. 5A-5C show that anti-MUC1 monoclonal antibody chBG138P, which targets the MUC1 membrane proximal region, binds to MUC1-positive cancer cell lines. FIGS. 5A-5C, show that chBG138P binds to MUC1-expressing tumor cell lines HCC827 (FIG. 5A), H1975 (FIG. 5B), and T-47D (FIG. 5C) in a dose dependent manner (with human IgG1 as a negative control).

FIG. 6 shows the schema of a FACS gating strategy for a T cell binding assay. The dashed boxes show the proportion of antibody-bound T cells.

FIGS. 7A-7H show that chimeric anti-MUC1 monoclonal antibodies targeting the MUC1 membrane proximal region, chBG138P (FIGS. 7A, 7B, 7E, and 7F), chBG219P (FIGS. 7E and 7F), or chBG346P (FIGS. 7E and 7F) do not bind to activated T cells, while MUC1 membrane distal portion targeting antibody HMFG1 (FIGS. 7C and 7D) and 16A (FIGS. 7G and 7H) can bind to normal T cells.

FIGS. 8A-8D depicts that chBP138P and humanized MUC1 antibodies, BG138P-hz2 and BG138P-hz4 (FIGS. 8A and 8B), do not bind to normal T cells, while MUC1 membrane distal portion targeting antibody HMFG1 (FIGS. 8C and 8D) binds to normal activated T cells.

FIGS. 9A-9C are bar graphs showing chBG138P-induced internalization of MUC1 in cancer cell lines.

FIGS. 10A and 10B show FACS binding of chimeric BG138P and its humanized antibodies huBG138P-Hz1 to huBG138P-Hz4 to human and cyno cells overexpressing MUC1.

FIG. 11 shows that humanized antibodies huBG219P-Bz0, −E39, and −E43 bind comparably to MUC1-overexpressing cell line ZR-75-1 comparably to compared with chimeric antibody chBG219P.

FIG. 12 shows the results of a binding competition assay between scFv1-78P, scFv2 (scFv2-14P, scFv2-57P), and scFv3-20P.

### DEFINITIONS

Unless specifically defined below or elsewhere in this document, all other technical and scientific terms used herein have the meaning commonly understood by one of ordinary skill in the art.

As used herein, including in the appended claims, the singular forms of words such as “a,” “an,” and “the” include their corresponding plural references unless the context clearly dictates otherwise.

The term “or” is used to mean, and is used interchangeably with, the term “and/or” unless the context clearly dictates otherwise.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within one or more than one standard deviation per the practice in the art. “About” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” should be assumed to be within an acceptable error range for that particular value or composition.

The term “MUC1” or “Mucin 1,” also known as CA 15-3, EMA, MCD, PEM, PUM, KL-6, MAM6, MCKD, PEMT, CD227, H23AG, MCKD1, ADMCKD, or ADTKD2, is one member of the mucin family. The amino acid sequence of human MUC1 is listed as SEQ ID NO: 1 and can also be found at accession number P15941.

The terms “administration,” and “administering,” as used herein, when applied to an animal, human, subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell.

The term “subject” or “patient” herein includes any organism, preferably an animal, more preferably a mammal (e.g., rat, mouse, dog, cat, rabbit, primate) and most preferably a human (e.g., a patient comprising, or at risk of having, a disorder described herein).

“Treating” any disease or disorder refers in one aspect to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically (e.g., stabilization of a discernible symptom), physiologically (e.g., stabilization of a physical parameter), or both.

The term “affinity” as used herein refers to the strength of interaction between antibody and antigen. Within the antigen, the variable regions of the antibody interact through non-covalent forces with the antigen at numerous sites. In general, the more interactions, the stronger the affinity.

The term “antibody,” (“Ab”), as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL or Vκ) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997); Lefranc, M.-P., The Immunologist, 7, 132-136 (1999); Lefranc, M.-P. et al., Dev. Comp. Immunol., 27, 55-77 (2003)).

The term “antibody” includes, but is not limited to, monoclonal antibodies, human antibodies, humanized antibodies, chimeric antibodies, anti-idiotypic (anti-Id) antibodies, and human engineered antibodies. The antibodies can be of any isotype/class (e.g., IgG, IgE, IgM, IgD, IgA and IgY), or subclass (e.g., IgG1, IgG2, IgG3, IgG4, IgA1 and IgA2).

The term “chimeric antibody” means molecules made up of domains from different species, i.e., fusing the variable domain of an antibody from one host species (e.g. mouse, rabbit, llama, etc.) with the constant domain of an antibody from a different species (e.g. human).

In some embodiments, the anti-MUC1 antibodies comprise at least one antigen-binding site. In some embodiments, the anti-MUC1 antibodies comprise an antigen-binding fragment from an MUC1 antibody described herein. In some embodiments, the anti-MUC1 antibody is isolated or recombinant. In some embodiments, the anti-MUC1 antibodies also encompass multi-specific antibodies targeting MUC1 as at least one arm and targeting other antigen(s) as another arm(s).

The term “monoclonal antibody” or “mAb” or “Mab” herein means a population of substantially homogeneous antibodies, i.e., the antibody molecules in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively.

Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (FR3), CDR-3 (CDR3), and FR-4 (FR4). The positions of the CDRs and framework regions can be determined using various well known definitions in the art, e.g., Kabat, Chothia, AbM, and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997) ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2), and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2, and LCDR3 in the light chain variable domain and HCDR1, HCDR2, and HCDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but are not limited to, Fab, Fab′, F(ab′)2, and Fv fragments; diabodies; linear antibodies; single-chain antibody molecules, e.g., single chain Fv (ScFv); nanobodies (or VHH antibody); multi-specific antibodies formed from antibody fragments; and bicyclic peptides (Hurov, K. et al., 2021. Journal for ImmunoTherapy of Cancer, 9(11)).

As used herein, an antibody or antigen-binding antibody fragment “specifically binds” to an antigen (e.g., a protein), meaning the antibody exhibits preferential binding to that target as compared to other proteins, but this specificity does not require absolute binding specificity. A “specific” or “selective” binding reaction is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten times when compared to the background level of binding and does not specifically bind in a significant amount to other antigens present in the sample.

“Antigen-binding domain” as used herein, comprises at least six CDRs (or three CDRs in terms of single domain antibody) and specifically binds to an epitope. An “antigen-binding domain” of a multi-specific antibody (e.g., a bispecific antibody) comprises a first antigen binding domain that specifically binds to a first epitope and a second antigen binding domain that specifically binds to a second epitope. Multi-specific antibodies can be bispecific, trispecific, tetraspecific, etc., with antigen binding domains directed to each specific epitope. Multi-specific antibodies can be multivalent (e.g., a bispecific tetravalent antibody) that comprises multiple antigen binding domains, for example, 2, 3, 4, or more antigen binding domains that specifically bind to a first epitope and 2, 3, 4, or more antigen binding domains that specifically bind to a second epitope.

The term “human antibody” herein means an antibody that comprises only human immunoglobulin protein sequences. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine, rabbit, llama, etc.) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental (e.g., rodent) antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable region, or other combinations of sequences or subsequences. Sequence identity can be determined using the methods described herein, for example, aligning two sequences using BLAST, ALIGN, or another alignment algorithm known in the art. The corresponding human germline nucleic acid or amino acid sequence can have at least about 90%, 91%, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant” or “K_{D} ” or “M” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The term “cancer” or “tumor” used herein has the broadest meaning as understood in the art and refers to the physiological condition in mammals that is typically characterized by unregulated cell growth. In the context of the present disclosure, the cancer is not limited to a certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g., its binding affinity to MUC1. Common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H}I interfaces or VH/VL interfaces of antibodies (see, e.g., US 2011/0287009, US2007/0178552, WO 96/027011, WO 98/050431, and Zhu et al., 1997, Protein Science, 6:781-788). In some embodiments, knob-into-holes ensure the correct pairing of two different heavy chains together during the manufacture of multi-specific antibodies. For example, multi-specific antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also ensure correct pairing.

Examples of algorithms that are suitable for determining percent sequence identity and sequence similarity are the BLAST algorithms, which are described in Altschul et al, Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89: 10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, (1988), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include anti-MUC1 antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination components.

The term “combination therapy” refers to the administration of two or more therapeutic agents to treat a therapeutic condition or disorder. Such administration encompasses co-administration of these therapeutic agents in a substantially simultaneous manner. Such administration also encompasses co-administration in multiple or in separate containers or formulations (e.g., capsules, powders, and liquids) for each active ingredient. Powders and/or liquids can be reconstituted or diluted to a desired dose prior to administration. In addition, “combination therapy” encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-MUC1 antibody is administered to the subject at the same time as, just before, or just after administration of an additional therapeutic agent. In certain embodiments, an anti-MUC1 antibody is administered as a co-formulation with an additional therapeutic agent.

### DETAILED DESCRIPTION

The present disclosure provides for anti-human MUC1 antibodies and antigen-binding fragments thereof. The present disclosure also provides for antibodies that have desirable binding affinity, desirable cellular internalization, and other desirable attributes. The anti-human MUC1 antibodies can be used to construct multi-specific antibodies with additional functionalities, such as binding a second human tumor associated antigen (TAA), immune checkpoint inhibition, or immune stimulation, to construct antibody drug conjugates (ADC), or to fuse with other domains to form fusion proteins. Furthermore, the anti-human MUC1 antibodies and the constructs thereof, or pharmaceutical compositions comprising the same, could be used for treating MUC1-expressing cancers and associated disorders.

### Anti-MUC1 Antibodies

The present disclosure provides for antibodies or antigen-binding fragments thereof that specifically bind to human MUC1. In embodiments, the antibodies or antigen-binding fragments thereof specifically bind to the SEA domain, which is a membrane proximal portion, of human MUC1 protein. In embodiments, the antibodies or antigen-binding fragments thereof have no or significantly reduced interference by soluble MUC1 compared with antibodies targeting the N terminal of MUC1. Antibodies or antigen-binding fragments of the present disclosure include, but are not limited to, the antibodies or antigen-binding fragments thereof produced by the methods described below.

In embodiments, the antibodies or antigen-binding fragments disclosed herein specifically bind to MUC1, and comprise a VH domain having an amino acid sequence of SEQ ID NO: 10, 20, 30, 57, 58, 61, 71, 75, or 80. In embodiments, the antibodies or antigen-binding fragments specifically bind to MUC1, and comprise a HCDR having an amino acid sequence of any one of the HCDRs listed in Tables 2, 7, 14, 16, 22 and 23, infra. In one aspect, the antibodies or antigen-binding fragments specifically bind to MUC1 and the antibodies comprise (or alternatively, consist of) one, two, three, or more HCDRs having an amino acid sequence of any of the HCDRs listed in Tables 2, 7, 14, 16, 22, and 23 herein.

In embodiments, the antibodies or antigen-binding fragments disclosed herein specifically bind to MUC1, and comprise a VL domain having an amino acid sequence of SEQ ID No: 11, 21, 31, 59, 60, 62, 66, 68, 72, 76, or 81. In embodiments, the antibodies or antigen-binding fragments specifically bind to MUC1, and comprise a LCDR having an amino acid sequence of any one of the LCDRs listed in Tables 2, 7, 14, 16, 22, and 23. In embodiments, the antibodies or antigen-binding fragments specifically bind to MUC1, and comprise (or alternatively, consist of) one, two, three, or more LCDRs having an amino acid sequence of any of the LCDRs listed in Tables 2, 7, 14, 16, 22, and 23.

In embodiments, the antibodies or antigen-binding fragments disclosed herein include amino acids that have been changed, yet have at least 60%, 70%, 80%, 90%, 95%, or 99% identity in the CDR regions with the CDR regions disclosed in Tables 2, 7, 14, 16, 22, and 23. In some aspects, it includes amino acid changes wherein no more than 1, 2, 3, 4, or 5 amino acids have been changed in the CDR regions when compared with the CDR regions depicted in the sequences in Tables 2, 7, 14, 16, 22, and 23.

In embodiments, the antibodies or antigen-binding fragments disclosed herein include those in which the amino acids or nucleic acids encoding the amino acids have been changed, yet have at least 60%, 70%, 80%, 90%, 95%, or 99% identity to the sequences disclosed in Tables 2, 7, 14, 16, 22, and 23. In some aspects, it includes changes in the amino acid sequences wherein no more than 1, 2, 3, 4, or 5 amino acids have been changed in the variable regions when compared with the variable regions depicted in the sequences disclosed in Tables 2, 7, 14, 16, 22, and 23, while retaining substantially the same therapeutic activity.

The present disclosure also provides nucleic acid sequences that encode VH, VL, the full length heavy chain, and the full length light chain of the antibodies that specifically bind to MUC1. Such nucleic acid sequences can be optimized for expression in mammalian cells.

In embodiments, the antibodies or antigen-binding fragments thereof disclosed herein have cross reactivity to both human and cynomolgus MUC1 (cyno MUC1, SEQ ID NO: 3). In embodiments, the antibodies or antigen-binding fragments target MUC1 membrane-proximal region with minimal interference from shed MUC1. In embodiments, the antibodies or antigen-binding fragments may be useful in the treatment of MUC1-expressing cancers.

The present disclosure also provides for antibodies and antigen-binding fragments thereof that bind to the same epitope as do the anti-MUC1 antibodies described in Tables 2, 7, 14, 16, 22, and 23. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to MUC1 demonstrates that the test antibody can compete with that antibody or antigen-binding fragments thereof for binding to MUC1. Such an antibody can, without being bound to any one theory, bind to the same or a related (e.g., a structurally similar or spatially proximal) epitope on MUC1 as the antibody or antigen-binding fragments thereof with which it competes. In a certain aspect, the antibody that binds to the same epitope on MUC1 as the antibodies or antigen-binding fragments thereof of the present disclosure is a human or humanized monoclonal antibody. Such human or humanized monoclonal antibodies can be prepared and isolated as described herein.

In one embodiment, the anti-MUC1 antibodies or antigen-binding fragments thereof as disclosed herein can be used to construct multi-specific antibodies with additional functionalities, such as binding a second human tumor associated antigen (TAA), immune checkpoint inhibition, or immune stimulation.

In some embodiments, the antibody or antigen-binding fragment thereof is a monoclonal antibody, a human engineered antibody, a single chain antibody (scFv), a Fab fragment, a Fab′ fragment, or a F(ab′)2 fragment.

In one embodiment, the antibody or antigen-binding fragment thereof is in a scFv format comprising VH-VL in the direction of N-terminal to C-terminal, or VL-VH in the direction of N-terminal to C-terminal. In some embodiments, the VH and VL are connected via an amino acid linker, such as an amino acid linker described herein. In some embodiments, the VH or VL is any one of VHs or VLs described in Tables 2, 7, 14, 16, 22, and 23. Other scFv of the present disclosure include amino acids that have been changed, yet have at least 60%, 70%, 80%, 90%, 95%, or 99% identity in the CDR regions with the CDR regions disclosed in Tables 2, 7, 14, 16, 22, and 23. In some aspects, it includes amino acid changes wherein no more than 1, 2, 3, 4, or 5 amino acids have been changed in the CDR regions when compared with the CDR regions depicted in the sequences in Tables 2, 7, 14, 16, 22, and 23.

### Amino Acid Linkers

In embodiments, the anti-MUC1 antibodies or antigen-binding fragments thereof disclosed herein are used to construct multi-specific antibodies, which may be, for example, bispecific tetravalent antibodies. The domains and/or regions of the polypeptide chains of bispecific tetravalent antibodies may be separated by linker regions of various lengths. In some embodiments, the antigen binding domains are separated from each other, a CL, CH1, hinge, CH2, CH3, or the entire Fc region by a linker region. For example, the polypeptide chains may include the sequence VL1-CL-(linker) VH2-CH1, VH-linker-VL. Such linker regions may comprise a random assortment of amino acids or a restricted set of amino acids. Such linker regions can be flexible or rigid (see US2009/0155275).

Multi-specific antibodies have been constructed by genetically fusing two single chain Fv (scFv) or Fab fragments with or without the use of flexible linkers (Mallender et al., J. Biol. Chem. 1994 269:199-206; Macket al., Proc. Natl. Acad. Sci. USA. 1995 92:7021-5; Zapata et al., Protein Eng. 1995 8.1057-62); via a dimerization device such as leucine zipper (Kostelny et al., J. Immunol. 1992148:1547-53; de Kruifetal J. Biol. Chem. 1996 271:7630-4) and Ig C/CH1 domains (Muller et al., FEBS Lett. 422:259-64); by diabody (Holliger et al., (1993) Proc. Nat. Acad. Sci. USA. 1998 90:6444-8; Zhu et al., Bio/Technology (NY) 1996 14:192-6); Fab-scFv fusion (Schoonjans et al., J. Immunol. 2000 165:7050-7); and mini antibody formats (Pack et al., Biochemistry 1992.31:1579-84; Pack et al., Bio/Technology 1993 11:1271-7).

### Dimerization-Specific Amino Acids

In one embodiment, the multivalent antibody comprises at least one dimerization-specific amino acid change. The dimerization-specific amino acid change may result in “knob-into-hole” interactions, and may increase the likelihood of correct assembly of desired multivalent antibodies. The dimerization-specific amino acids may be within the CH1 domain or the CL domain or combinations thereof. Suitable dimerization-specific amino acids used to pair CH1 domains with other CH1 domains (CH1-CH1) and CL domains with other CL domains (CL-CL) may be found at least in the disclosures of WO2014082179, WO2015181805, and WO2017059551. The dimerization-specific amino acids can also be within the Fc domain and can be in combination with dimerization-specific amino acids within the CH1 or CL domains. In one embodiment, the present disclosure provides a bispecific antibody comprising at least one dimerization-specific amino acid pair.

### Further Alteration of the Framework of Fc Region

In aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII, and FcRn have been mapped and variants with improved binding have been described (see Shields et al., J. Biol. Chem. 276:6591-6604, 2001).

In still another aspect, the glycosylation of the antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for an antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lec13 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures, which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G L, et al., 2010 MAbs, 2:181-189). However, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of an antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel S M, et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al, 2000 Nature Medicine, 6:443-446; Arnold J. N., 2007 Annu Rev immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate antibodies with low ADCC and CDC but with good stability, it is possible to modify the hinge and Fc region of human IgG4 and introduce a number of alterations. These modified IgG4 Fc molecules can be found in SEQ ID NOs: 83-88, U.S. Pat. No. 8,735,553 to Li et al.

### Antibody Production

Antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The present disclosure further provides polynucleotides encoding the antibodies described herein, e.g., polynucleotides encoding heavy or light chain variable regions or segments comprising the complementarity determining regions as described herein.

The polynucleotides of the present disclosure can encode the variable region sequence of an anti-MUC1 antibody. They can also encode both a variable region and a constant region of the antibody. Some of the sequences encode a polypeptide that comprises variable regions of both the heavy chain and the light chain of the exemplified anti-MUC1 antibodies.

Also provided in the present disclosure are expression vectors and host cells for producing the anti-MUC1 antibodies. The choice of expression vector depends on the intended host cells in which the vector is to be expressed. The expression vectors may contain a promoter and other regulatory sequences (e.g., enhancers) that are operably linked to the polynucleotides encoding an anti-MUC1 antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be included for efficient expression of an anti-MUC1 antibody or antigen-binding fragment thereof. These elements may include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-MUC1 antibody vectors can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters may be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-MUC1 antibodies. Insect cells in combination with baculovirus vectors can also be used.

In other aspects, mammalian host cells are used to express and produce the anti-MUC1 antibodies of the present disclosure. Examples include a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Methods of Detection and Diagnosis

The antibodies or antigen-binding fragments of the present disclosure are useful in a variety of applications including, but not limited to, methods for the detection of MUC1. In one aspect, the antibodies or antigen-binding fragments are useful for detecting the presence of MUC1 in a biological sample. The term “detecting” as used herein includes quantitative or qualitative detection. In certain aspects, a biological sample comprises a cell or tissue. In other aspects, such tissues include normal and/or cancerous tissues that express MUC1 at higher levels relative to other tissues.

In one aspect, the present disclosure provides a method of detecting the presence of MUC1 in a biological sample. In certain aspects, the method comprises contacting the biological sample with an anti-MUC1 antibody under conditions permissive for binding of the antibody to an antigen and detecting whether a complex is formed between the antibody and the antigen. The biological sample can include, without limitation, urine, tissue, sputum, or blood.

Also included is a method of diagnosing a disorder associated with expression of MUC1. In certain aspects, the method comprises contacting a test cell with an anti-MUC1 antibody; determining the level of expression (either quantitatively or qualitatively) of MUC1 expressed by the test cell by detecting binding of the anti-MUC1 antibody to the MUC1 polypeptide; and comparing the level of expression by the test cell with the level of MUC1 expression in a control cell (e.g., a normal cell of the same tissue origin as the test cell or a non-MUC1 expressing cell), wherein a higher level of MUC1 expression in the test cell as compared to the control cell indicates the presence of a disorder associated with expression of MUC1.

### Pharmaceutical Compositions and Formulations

Also provided are compositions, including pharmaceutical formulations, comprising an anti-MUC1 antibody or antigen-binding fragment thereof, or polynucleotides comprising sequences encoding an anti-MUC1 antibody or antigen-binding fragment. These compositions can further comprise suitable carriers, such as pharmaceutically acceptable excipients including buffers, which are well known in the art.

Pharmaceutical formulations of an anti-MUC1 antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Nos. U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility can be readily accomplished, e.g., by filtration through sterile filtration membranes.

### Equivalents

It is to be understood that, while the anti-human 4Ig-B7H3 antibodies and antigen binding fragments thereof have been described in conjunction with a detailed description thereof, the foregoing description is intended to illustrate and not limit the scope of the invention, which is defined by the scope of the appended claims. Other aspects, advantages, and modifications are within the scope of the claims below.

It is to be understood that one, some, any, or all of the features of the various embodiments disclosed herein may be combined to form further embodiments of the present disclosure. These and other aspects of the present disclosure will be apparent to those skilled in the art.

### EXAMPLES

### Example 1. Generation of Anti-MUC1 Monoclonal Antibodies Targeting MUC1 Membrane-Proximal Region

### MUC1 Recombinant Protein for Immunization and Binding Assays

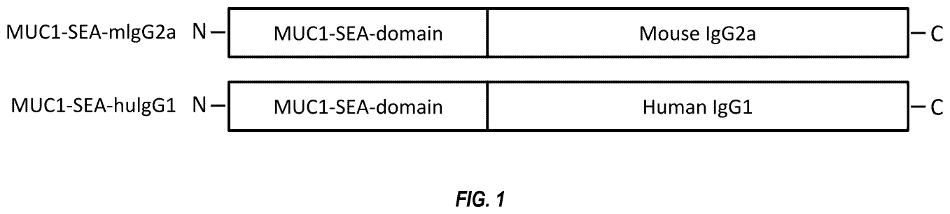

The cDNA coding for the full-length human MUC1 (SEQ ID NO: 1) was synthesized by and purchased from Genewiz (Suzhou, China) based on its Uniprot sequence (UniProtKB: P15941). The coding region of the SEA domain (Sea-urchin Sperm Protein, Enterokinase and Agrin) (SEQ ID NO: 2) of the full-length human MUC1, which consisted of amino acids (AA) 1036-1155 of the full-length MUC1, was PCR-amplified and cloned into pcDNA3.4-based expression vector (Invitrogen, Carlsbad, CA, USA) with the C-terminus fused either to the Fc domain of mouse IgG2a or to the Fc domain of human IgG1 heavy chain, which resulted in two recombinant fusion protein expression plasmids, MUC1-SEA-mIgG2a and MUC1-SEA-huIgG1, respectively. A schematic depiction of MUC1 fusion proteins is shown in FIG. 1. For the recombinant fusion protein production, MUC1-SEA-mIgG2a and MUC1-SEA-huIgG1 plasmids were transiently transfected into Expi293 cells (Thermo Fisher, Waltham, MA, USA) and cultured for 6 days in a CO₂ incubator equipped with a rotating shaker. The supernatant containing the recombinant proteins was collected and cleared by centrifugation. MUC1-SEA-mIgG2a and MUC1-SEA-huIgG1 were purified using a Protein A column (Cat.: 17549852, Cytiva Life Sciences) followed by HiLoad 16/600 Superdex 200 pg size exclusion column (Cat.: 28989335, Cytiva Life Sciences). Both MUC1-SEA-mIgG2a and MUC1-SEA-huIgG1 proteins were dialyzed against phosphate buffered saline (PBS) and stored in a −80° C. freezer in small aliquots.

TABLE 1
Sequences of MUC1

| | SEQ ID NO. | SEQUENCE |
|---|---|---|
| Human | SEQ ID NO: 1 | MTPGTQSPFFLLLLLTVLTVVTGSGHASSTPGGEKETS |
| MUC1 | | ATQRSSVPSSTEKNAVSMTSSVLSSHSPGSGSSTTQGQ |
| (full | | DVTLAPATEPASGSAATWGQDVTSVPVTRPALGSTTPP |
| length) | | AHDVTSAPDNKPAPGSTAPPAHGVTSAPDTRPAPGSTA |
| | | PPAHGVTSAPDTRPAPGSTAPPAHGVTSAPDTRPAPGS |
| | | TAPPAHGVTSAPDTRPAPGSTAPPAHGVTSAPDTRPAP |
| | | GSTAPPAHGVTSAPDTRPAPGSTAPPAHGVTSAPDTRP |
| | | APGSTAPPAHGVTSAPDTRPAPGSTAPPAHGVTSAPDT |
| | | RPAPGSTAPPAHGVTSAPDTRPAPGSTAPPAHGVTSAP |
| | | DTRPAPGSTAPPAHGVTSAPDTRPAPGSTAPPAHGVTS |
| | | APDTRPAPGSTAPPAHGVTSAPDTRPAPGSTAPPAHGV |
| | | TSAPDTRPAPGSTAPPAHGVTSAPDTRPAPGSTAPPAH |
| | | GVTSAPDTRPAPGSTAPPAHGVTSAPDTRPAPGSTAPP |
| | | AHGVTSAPDTRPAPGSTAPPAHGVTSAPDTRPAPGSTA |
| | | PPAHGVTSAPDTRPAPGSTAPPAHGVTSAPDTRPAPGS |
| | | TAPPAHGVTSAPDTRPAPGSTAPPAHGVTSAPDTRPAP |
| | | GSTAPPAHGVTSAPDTRPAPGSTAPPAHGVTSAPDTRP |
| | | APGSTAPPAHGVTSAPDTRPAPGSTAPPAHGVTSAPDT |
| | | RPAPGSTAPPAHGVTSAPDTRPAPGSTAPPAHGVTSAP |
| | | DTRPAPGSTAPPAHGVTSAPDTRPAPGSTAPPAHGVTS |
| | | APDTRPAPGSTAPPAHGVTSAPDTRPAPGSTAPPAHGV |
| | | TSAPDTRPAPGSTAPPAHGVTSAPDTRPAPGSTAPPAH |
| | | GVTSAPDTRPAPGSTAPPAHGVTSAPDTRPAPGSTAPP |
| | | AHGVTSAPDTRPAPGSTAPPAHGVTSAPDTRPAPGSTA |
| | | PPAHGVTSAPDTRPAPGSTAPPAHGVTSAPDNRPALGS |
| | | TAPPVHNVTSASGSASGSASTLVHNGTSARATTTPASK |
| | | STPFSIPSHHSDTPTTLASHSTKTDASSTHHSSVPPLTSS |
| | | NHSTSPQLSTGVSFFFLSFHISNLQFNSSLEDPSTDYYQ |
| | | ELQRDISEMFLQIYKQGGFLGLSNIKFRPGSVVVQLTL |
| | | AFREGTINVHDVETQFNQYKTEAASRYNLTISDVSVS |
| | | DVPFPFSAQSGAGVPGWGIALLVLVCVLVALAIVYLIA |
| | | LAVCQCRRKNYGQLDIFPARDTYHPMSEYPTYHTHGR |
| | | YVPPSSTDRSPYEKVSAGNGGSSLSYTNPAVAATSANL |
| Human | SEQ ID NO: 2 | LSTGVSFFFLSFHISNLQFNSSLEDPSTDYYQELQRDIS |
| MUC1 | | EMFLQIYKQGGFLGLSNIKFRPGSVVVQLTLAFREGTI |
| SEA | | NVHDVETQFNQYKTEAASRYNLTISDVSVSDVPFPFS |
| domain | | AQSGAG |
| Cyno | SEQ ID NO: 3 | MTPGTQSPFFLLLILTVLTAATVPEPTTVVTGSGHTNST |
| MUC1 | | PGGEKETSATQRSSMPISTKNAVSMTSRLSSHSPVSGS |
| (Full | | STTQGQDVTLALAMESATGSATTLGHVVTSAPDTSAA |
| length) | | PGSTGPPAHVVTSAPDTSAAPGSTGPPAHVVTSAPDTV |
| | | TSASDSASGSASTLVHSTTSARATTTPASKSTPFSIPSH |
| | | HSDTPTTLASHSTKTDASSTHHSTVPPFTSSNHSTSPQL |
| | | SLGVSFFFLSFHISNLQFNSSLEDPSTNYYQQLQRDISE |
| | | LFLQIYKQGDFLGLSNIMFRPGSVVVQSTLVFREGTTN |
| | | VHDVETQFNQRKTEAASRYNLTISDISVRDVPFPFSAQ |
| | | TGAGVPGWGIALLVLVCVLVVMAIVYFIALAVCQCRQ |
| | | KNYRQLDIFPARDAYHPMSEYPTYHTHGRYAPAGGTN |
| | | RSPYEEVSAGNGGSSLSYTNPAVAATSANL |

### Cell Lines Stably Expressing Human or Cyno MUC1 Were Generated for Antibody Generation, Screening, and Validation

Cell lines stably expressing human MUC1, including PT67/human MUC1 cell line (an internally generated cell line), HEK293/human MUC1 cell line (HEK293 is obtained from ATCC, CRL-1573), and HCT116/human MUC1 cell line (ATCC CCL-247) were generated and validated.

Cell lines stably expressing cyno MUC1 (SEQ ID NO: 3), including HEK293/cyno MUC1, L929/cyno MUC1 cell line (L929 was obtained from ATCC, CCL-1), HCT116/cyno MUC1 cell line, and Daudi/cyno MUC1 cell line (Daudi was obtained from ATCC, CCL-213), were generated and validated.

To generate cell lines that stably express human or cyno MUC1, ectotrophic vectors were constructed using retroviral construct PFBneo (STRATAGENE, cat. #217561-51). Retroviral construct transfection into PLAT-E cells (Cyagen, Cat. #IPMPC-01001) was performed using Lipofectamine 2000 (Invitrogen, REF #52758) according to the manufacturer's instructions. Viral supernatants were collected 24, 48 and 72 hours after transfection and filtered (0.45 μm) before use. The ectotrophic viruses produced above were used to transduce the dual tropic packaging cell line PT67 in the presence of polybrene (final concentration: 8 μg/ml). After 3 rounds of transduction, PT67 cells were selected in G418 (final concentration: 1 mg/ml) for 7 days. For collection of dual tropic viruses generated from PT67 cells, when cells became 100% confluent, medium were changed to fresh DMEM complete medium without G418. Viruses were collected once a day for 3 days. Cell lines were infected with virus that contain human or cyno MUC1. After 3 rounds of transduction, infected cells were selected in G418 (final concentration: 1 mg/ml) for 7 days.

### Immunization

To generate antibodies against MUC1, cohorts of 30 BALB/C, MRL strains of inbred mice were immunized with different MUC1 antigens with each cohort being subjected to an immunization strategy comprising a unique combination of MUC1 antigens (including the protein and cell line from Example 1), dose, injection route, adjuvant, and immunization timing. A total of 5 animals in 6 cohorts were immunized. Animals received immunizations over varying periods between 0 and 90 days. To monitor immune responses, titrated serum was screened by ELISA and FACS, typically after 30-90 days of 2-6 immunizations. Serum was screened for antibody binding to MUC1 antigens. MUC1-specific antibody responses were measured in each animal, and animals with sufficient titers of anti-MUC1 Ig were selected for final boost 4 days.

### Hybridoma Fusion and Screening

Lymphoid organs, including spleens and lymph nodes, were isolated from mice immunized as described above. Hybridomas were generated by fusions with immortalized mouse myeloma cells derived from the SP2/0 by PEG-based fusion. The resulting cells were plated in 96-well cell culture plates using regular 1640 medium supplemented with HAT for selection of hybridomas. After 10-13 days of culture and growth media replacement, hybridoma culture supernatants were collected from individual wells and screened to identify wells with secreted MUC1-specific antibodies. All supernatants were initially screened against recombinant proteins huMUC1-SEA-huIgG1 (from Example 1). Antibody binding on recombinant proteins huMUC1-SEA-huIgG1 were measured through ELISA. Supernatants from culture wells in 3 hybridoma fusions were screened for MUC1 antibodies. Briefly, 2 μg/mL huMUC1-SEA-huIgG1 was coated in the 96 well ELISA plates, and 50 μl of hybridoma culture supernatant were co-incubated for 30-60 min, washed, and incubated with anti-mouse IgG Fc secondary Ab conjugated to HRP. After incubation and washing, the plates were developed with HRP substrate and absorbance was measured.

Hybridomas from positive wells were transferred to 24-well plates with fresh culture media to grow for 2-3 days before screening again by flow cytometry to confirm antibodies binding to human MUC1 and cyno MUC1 overexpression cell lines.

Antibodies (Abs) binding to human MUC1 and cyno MUC1 overexpression cell lines were measured through FACS. Briefly, 100 μl of hybridoma culture supernatant and human MUC1 overexpression cells or cyno MUC1 overexpression cells were co-incubated for 30-60 min, washed, and incubated with anti-mouse IgG Fc secondary Ab conjugated to APC. After incubation and washing, fluorescence was measured by flow cytometry.

### Subcloning and Sequence Analysis

Selected anti-MUC1 Ab-secreting hybridomas were subcloned once or twice to ensure monoclonality. Briefly, the positive hybridoma clones were sub-cloned by limiting dilution. After 7-10 days, culture supernatant was screened by ELISA and flow cytometry as previously described to confirm human and cyno MUC1 Ab binding. Stable hybridoma subclones were cultured in vitro for cell cryopreservation and antibody VH and VL gene cloning and sequencing.

The anti-MUC1 Ab-secreting hybridomas after subcloning were lysed by lysis buffer. The mRNA-containing lysates were subsequently transferred to 96-well deep well plates for mRNA isolation, cDNA synthesis, and DNA sequencing by standard sequencing techniques (Sanger sequencing). In general, total RNA of the cell lysates was prepared and the cDNA was generated by reverse transcription of the mRNA using the Super Script III first-strand synthesis SuperMix (Invitrogen) according to the manufacturer's instructions. The sequences of the BG138P antibody are listed in Table 2.

### Single B Screening

Immunized mice were sacrificed and spleen were harvested. Enriched plasma cells were loaded onto 14K chip. hMUC1 beads, cynoMUC1 beads and HEK293-cynoMUC1 cells were used for on-chip screening. Hits were selected and exported into lysis buffer. Single cell RNA was purified and Ig sequence were recovered using BLI cDNA Synthesis Kit (BERKELEY LIGHTS) according to the manufacturer's instructions. The sequences of BG219P and BG346P are listed in Table 2.

TABLE 2
Sequences for murine antibodies against human MUC1

| Antibody | SEQ ID NO | Note | Sequence |
|---|---|---|---|
| BG138P | SEQ ID NO: 4 | HCDR1 | TYTMH |
| | | (Kabat) | |
| | SEQ ID NO: 5 | HCDR2 | YINPGSDYTHYNQKFKD |
| | | (Kabat) | |
| | SEQ ID NO: 6 | HCDR3 | QGNYAAYYFDY |
| | | (Kabat) | |
| | SEQ ID NO: 7 | LCDR1 | RASGNIHNFLV |
| | | (Kabat) | |
| | SEQ ID NO: 8 | LCDR2 | NAKTLTD |
| | | (Kabat) | |
| | SEQ ID NO: 9 | LCDR3 | QNFWNVPFT |
| | | (Kabat) | |
| | SEQ ID NO: 10 | VH AA | QVQLLQSGAELARPGASVKMSCKAS |
| | | | GYTFTTYTMHWIKQRPGQGLEWIGYI |
| | | | NPGSDYTHYNQKFKDKATLTADKSSS |
| | | | TAYMHLYSLTSEDSAVYYCARQGNYA |
| | | | AYYFDYWGQGTTLTVSS |
| | SEQ ID NO: 11 | VL AA | DIQMTQSPASLSASVGETVTITCRASG |
| | | | NIHNFLVWYQQKQGKSPQLLVYNAK |
| | | | TLTDGVPSRFSGSGSGTQYSLKIKSLQ |
| | | | PEDFGTYYCQNFWNVPFTFGSGTKLE |
| | | | IK |
| | SEQ ID NO: 12 | VH DNA | CAGGTCCAGTTGTTACAGTCTGGGG |
| | | | CTGAACTGGCAAGACCTGGGGCCTC |
| | | | AGTGAAGATGTCCTGCAAGGCTTCT |
| | | | GGTTACACCTTTACTACCTACACGAT |
| | | | GCACTGGATAAAACAGAGGCCTGGG |
| | | | CAGGGTCTGGAATGGATTGGATACAT |
| | | | TAATCCTGGCAGTGATTATACTCATTA |
| | | | CAATCAGAAATTCAAAGACAAGGCC |
| | | | ACATTGACTGCAGACAAATCCTCCA |
| | | | GCACAGCCTACATGCACCTGTACAG |
| | | | CCTGACATCTGAGGACTCTGCAGTC |
| | | | TATTACTGTGCAAGACAGGGCAACT |
| | | | ATGCCGCGTACTACTTTGACTACTGG |
| | | | GGCCAAGGCACCACTCTCACAGTCT |
| | | | CCTCA |
| | SEQ ID NO: 13 | VL DNA | GACATCCAGATGACTCAGTCTCCAG |
| | | | CCTCCCTATCTGCATCTGTGGGAGAA |
| | | | ACTGTCACCATCACATGTCGAGCAA |
| | | | GTGGGAATATTCACAATTTTTTAGTAT |
| | | | GGTATCAGCAGAAACAGGGAAAATC |
| | | | TCCTCAGCTCCTGGTCTATAATGCAA |
| | | | AAACCTTAACAGATGGTGTGCCATC |
| | | | AAGGTTCAGTGGCAGTGGATCAGGA |
| | | | ACACAATATTCTCTCAAGATCAAGAG |
| | | | CCTGCAGCCTGAAGATTTTGGGACT |
| | | | TATTACTGTCAAAATTTTTGGAATGT |
| | | | TCCATTCACGTTCGGCTCGGGGACA |
| | | | AAGTTGGAAATAAAA |
| BG346P | SEQ ID NO: 14 | HCDR1 | NYWMN |
| | | (Kabat) | |
| | SEQ ID NO: 15 | HCDR2 | EIDPSDSYTNNNQKFKD |
| | | (Kabat) | |
| | SEQ ID NO: 16 | HCDR3 | GRRTGFAY |
| | | (Kabat) | |
| | SEQ ID NO: 17 | LCDR1 | RASENIYSNLA |
| | | (Kabat) | |
| | SEQ ID NO: 18 | LCDR2 | AATNLAD |
| | | (Kabat) | |
| | SEQ ID NO: 19 | LCDR3 | QHFWGTPYT |
| | | (Kabat) | |
| | SEQ ID NO: 20 | VH AA | EVQLQQSGAELVKPGASVKISCKASG |
| | | | YTFTNYWMNWVRQRPGQGLEWIGEI |
| | | | DPSDSYTNNNQKFKDKATLTVDKSSS |
| | | | TAYMQLSSLTSEDSAVYYCARGRRTG |
| | | | FAYWGQGTLVTVSS |
| | SEQ ID NO: 21 | VL AA | DIQMTQSPASLSVSVGETVTITCRASE |
| | | | NIYSNLAWYQKKQGKSPQLLVYAATN |
| | | | LADGVPSRFSGSGSGTQYSLKINSLQS |
| | | | EDFGNYYCQHFWGTPYTFGGGTKLEI |
| | | | K |
| | SEQ ID NO: 22 | VH DNA | GAGGTCCAGCTTCAGCAGTCTGGGG |
| | | | CTGAGCTGGTGAAGCCTGGGGCTTC |
| | | | AGTGAAGATATCCTGCAAGGCGTCT |
| | | | GGCTACACGTTCACCAACTACTGGAT |
| | | | GAACTGGGTGAGGCAGAGGCCTGG |
| | | | ACAAGGCCTTGAGTGGATCGGAGAG |
| | | | ATCGATCCTTCTGATAGTTATACTAAC |
| | | | AACAATCAAAAGTTCAAGGACAAGG |
| | | | CCACATTGACTGTAGACAAATCCTCC |
| | | | AGCACAGCCTACATGCAGCTCAGCA |
| | | | GCCTGACATCTGAGGACTCTGCGGT |
| | | | CTATTACTGTGCAAGAGGGAGGAGG |
| | | | ACGGGGTTTGCTTACTGGGGCCAAG |
| | | | GGACTCTGGTCACTGTCTCGAGC |
| | SEQ ID NO: 23 | VL DNA | GACATCCAGATGACCCAGTCTCCAG |
| | | | CCTCCCTATCTGTATCTGTGGGAGAA |
| | | | ACTGTCACCATCACATGTCGAGCAA |
| | | | GTGAGAATATTTACAGTAATTTAGCA |
| | | | TGGTATCAGAAGAAACAGGGAAAAT |
| | | | CTCCTCAGCTCCTGGTCTATGCTGCA |
| | | | ACAAACTTAGCAGATGGTGTGCCAT |
| | | | CAAGGTTCAGTGGCAGTGGATCAGG |
| | | | CACACAGTATTCCCTCAAGATCAAC |
| | | | AGCCTGCAGTCTGAAGATTTTGGGA |
| | | | ATTATTACTGTCAACATTTTTGGGGT |
| | | | ACTCCGTACACGTTCGGAGGGGGGA |
| | | | CCAAGCTGGAAATAAAA |
| BG219P | SEQ ID NO: 24 | HCDR1 | DYIMS |
| | | (Kabat) | |
| | SEQ ID NO: 25 | HCDR2 | TVSSGGDNTYYPDSVKG |
| | | (Kabat) | |
| | SEQ ID NO: 26 | HCDR3 | YGGLGAMDY |
| | | (Kabat) | |
| | SEQ ID NO: 27 | LCDR1 | RASGNIHNYLT |
| | | (Kabat) | |
| | SEQ ID NO: 28 | LCDR2 | NAKTLAD |
| | | (Kabat) | |
| | SEQ ID NO: 29 | LCDR3 | QHFWSVPYT |
| | | (Kabat) | |
| | SEQ ID NO: 30 | VH AA | EVQLVESGGGLVKPGGSLKLSCAASG |
| | | | FTFNDYIMSWVRQTPEKRLEWVATVS |
| | | | SGGDNTYYPDSVKGRFTISRDNAKNN |
| | | | LYLQMSSLRSEDTALYYCTRYGGLGA |
| | | | MDYWGQGTSVTVSS |
| | SEQ ID NO: 31 | VL AA | DIVMTQSPASLSASVGETVTITCRASG |
| | | | NIHNYLTWYQQKQGKSPQLLVYNAK |
| | | | TLADGVPSRFSGSGSGPQYSLKINSLQ |
| | | | PEDFGSYYCQHFWSVPYTFGGGTRLE |
| | | | IK |
| | SEQ ID NO: 32 | VH DNA | GAAGTGCAGCTGGTGGAGTCTGGGG |
| | | | GAGGCTTAGTGAAGCCTGGAGGGTC |
| | | | CCTGAAACTCTCCTGTGCAGCCTCT |
| | | | GGATTCACTTTCAATGACTATATCATG |
| | | | TCTTGGGTTCGCCAGACTCCGGAGA |
| | | | AGAGGCTGGAGTGGGTCGCAACCGT |
| | | | TAGTAGTGGTGGTGATAACACCTACT |
| | | | ATCCAGATAGTGTGAAGGGTCGATTC |
| | | | ACCATCTCCAGAGACAATGCCAAGA |
| | | | ACAACCTGTACCTGCAAATGAGCAG |
| | | | TCTGAGGTCTGAGGACACGGCCTTG |
| | | | TATTACTGTACAAGATATGGGGGGTT |
| | | | AGGGGCTATGGACTACTGGGGTCAA |
| | | | GGAACCTCAGTCACCGTCTC |
| | SEQ ID NO: 33 | VL DNA | GACATTGTGATGACCCAGTCTCCAG |
| | | | CCTCCCTATCTGCATCTGTGGGAGAA |
| | | | ACTGTCACCATCACATGTCGAGCAA |
| | | | GTGGGAATATTCACAATTATTTAACA |
| | | | TGGTATCAGCAGAAACAGGGAAAAT |
| | | | CTCCTCAGCTCCTGGTCTATAATGCA |
| | | | AAAACCTTAGCAGATGGTGTGCCAT |
| | | | CAAGGTTCAGTGGCAGTGGATCAGG |
| | | | ACCACAATATTCTCTCAAGATCAACA |
| | | | GCCTGCAGCCTGAAGATTTTGGGAG |
| | | | TTATTACTGTCAACATTTTTGGAGTG |
| | | | TTCCGTACACGTTCGGAGGGGGGAC |
| | | | CAGACTGGAAATAAAA |

### Large Scale Expression and Purification of Chimeric BG219P, BG138P, and BG346P

The chimeric antibodies chBG219P, chBG138P, and chBG346P were produced by transient transfection of in-house generated heavy and light chain containing plasmids to ExpiCHO-s cells. The conditioned media was harvested and the antibodies were purified using a MabSelect SuRe column (Cytiva) followed by a POROS™ 50 HS column (Thermofisher Scientific) and a G-25 desalting column (Cytiva). All the purified antibodies were stored in −80° C. freezer in small aliquots.

### Example 2. Binding Kinetics and Affinity Determination of Anti-MUC1 Antibodies by SPR

Chimeric anti-MUC1 antibodies were characterized for binding kinetics by SPR assays using BIAcore™ T-200 (GE Life Sciences). Briefly, mouse anti-human IgG Fc antibody was immobilized on an activated CM5 biosensor chip (Cat. No. BR100530, GE Life Sciences). Purified chimeric anti-MUC1 antibodies were flowed over the chip surface and captured by anti-human IgG antibody. Then a serial dilution of human or cyno MUC1-SEA protein was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding affinity profiles of chimeric anti-MUC1 antibodies chBG138P, chBG346P, and chBG219P are shown below in Table 3. ChBG138P, chBG346P, and chBG219P displayed high affinity towards both human and cyno MUC1-SEA.

TABLE 3
Antigen binding affinity of chBG138P, chBG346P, and chBG219P

| Ab # | Antigen | ka (1/Ms) | kd (1/s) | KD (M) |
|---|---|---|---|---|
| chBG138P | HuMUC1-SEA | 2.37E+05 | 4.37E−04 | 1.85E−09 |
| | CynoMUC1-SEA | 6.38E+05 | 9.75E−03 | 1.53E−08 |
| chBG346P | HuMUC1-SEA | 7.97E+05 | 7.59E−04 | 9.53E−10 |
| | CynoMUC1-SEA | 2.67E+05 | 3.35E−04 | 1.26E−09 |
| chBG219P | HuMUC1-SEA | 3.39E+05 | 1.25E−04 | 3.69E−10 |
| | CynoMUC1-SEA | 3.52E+05 | 3.29E−04 | 9.34E−10 |

### Example 3. Determining the Binding Affinity of Anti-MUC1 Antibodies to MUC1 Expressed in Stable Expression Cell Lines

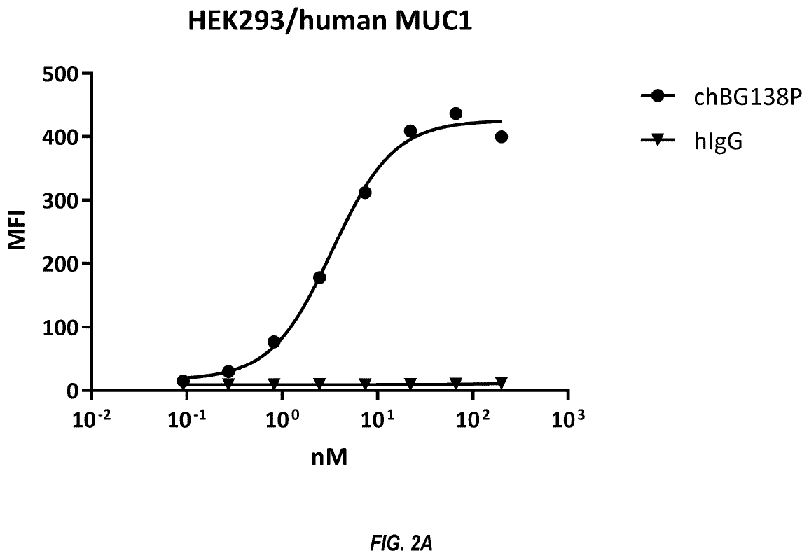

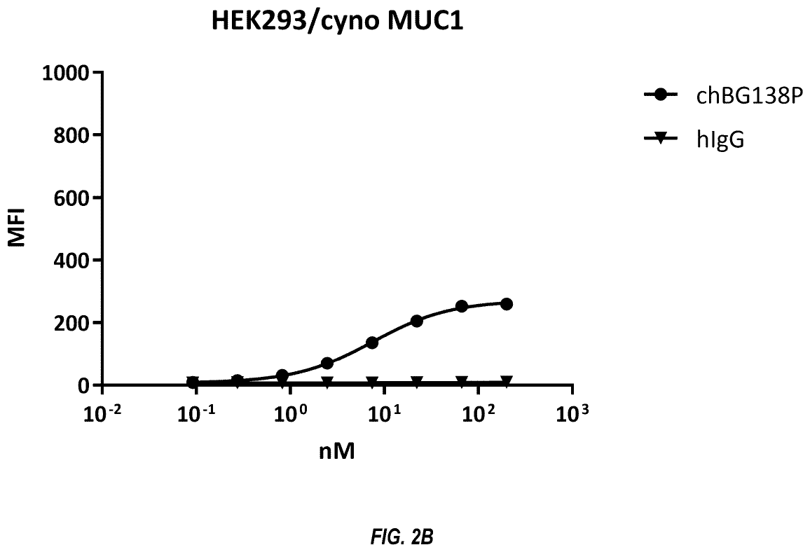

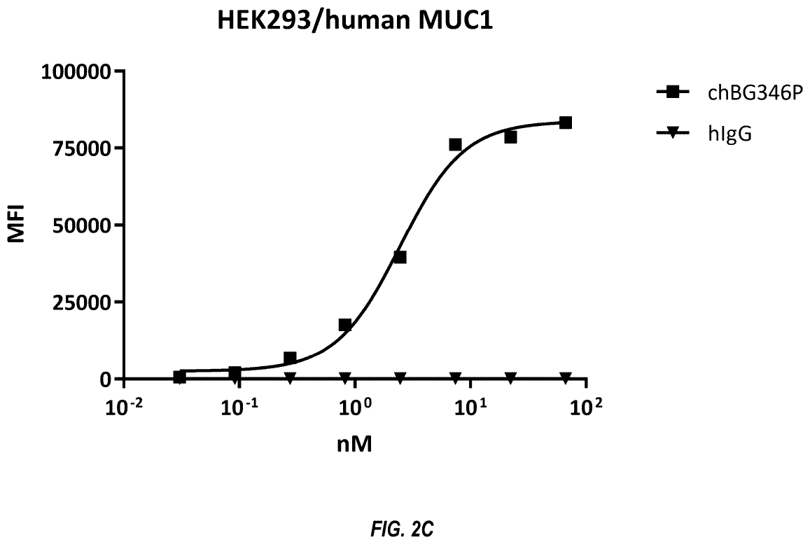

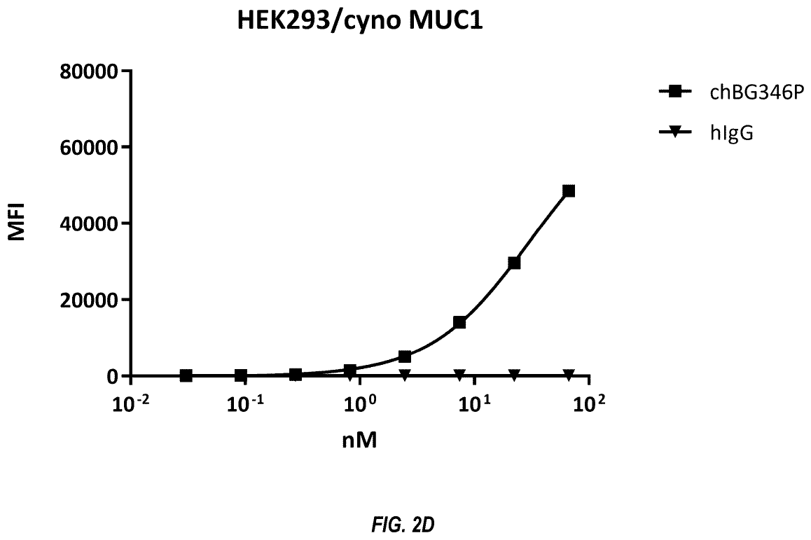

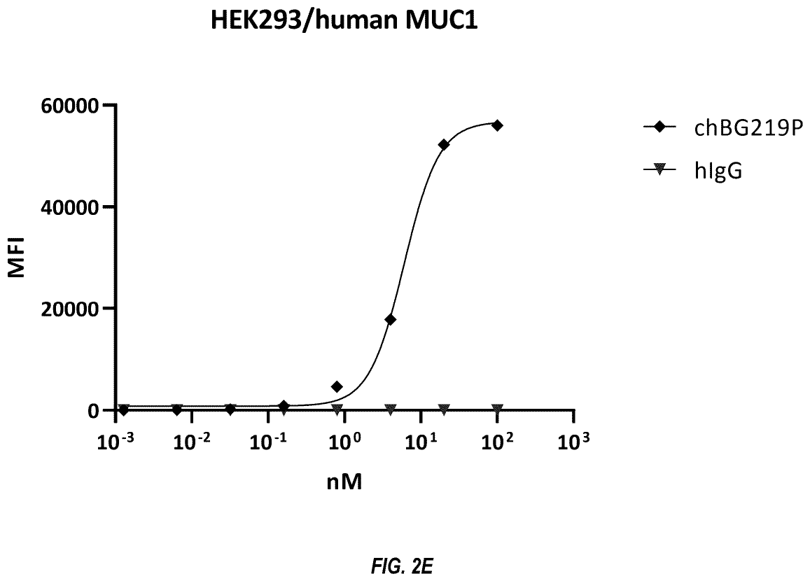

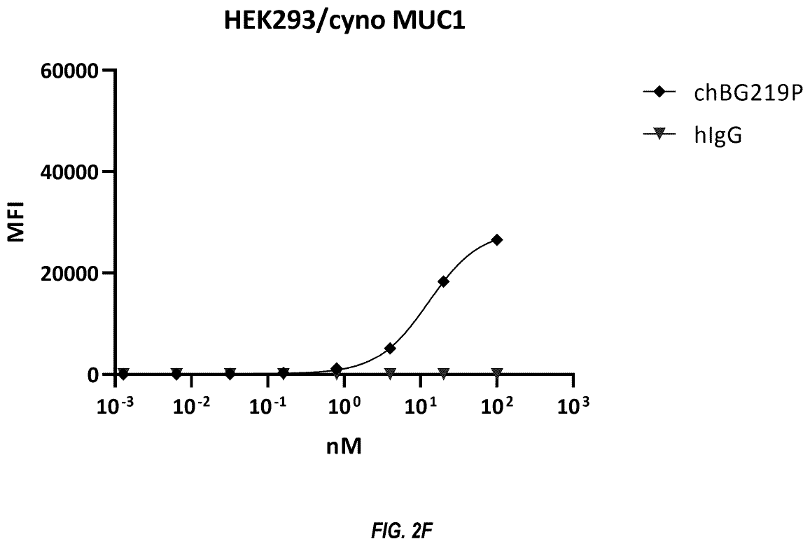

The binding affinity of chimeric anti-MUC1 antibodies to human and cyno MUC1 overexpression cell lines (HEK293/human MUC1 and HEK293/cyno MUC1) by FACS was determined. Briefly, human or cyno MUC1 overexpression cells were incubated with serially diluted purified antibodies, washed, and incubated with anti-human IgG secondary antibody conjugated to APC. After incubation and washing, fluorescence was measured by flow cytometry. The binding affinity profiles of chimeric anti-MUC1 antibodies are shown below in Table 4 and FIGS. 2A-2F (with hIgG1 as negative control). The results demonstrate that all three chimeric anti-MUC1 antibodies have favorable binding affinity to both human and cyno MUC1 expressed in stable expression cell lines.

TABLE 4
Cell binding affinity of chBG138P, chBG346P, and chBG219P

| Ab # | Cell line | EC₅₀ (nM) |
|---|---|---|
| chBG138P | Human MUC1 | 3.37 |
| | Cyno MUC1 | 7.55 |
| chBG346P | Human MUC1 | 3.01 |
| | Cyno MUC1 | 41.76 |
| chBG219P | Human MUC1 | 6.06 |
| | Cyno MUC1 | 12.71 |

### Example 4. Epitope Binning of Anti-MUC1 Antibodies

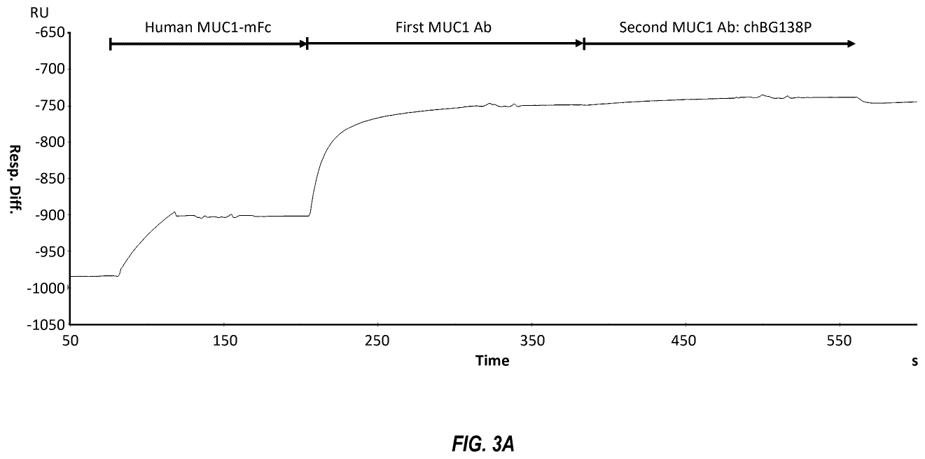

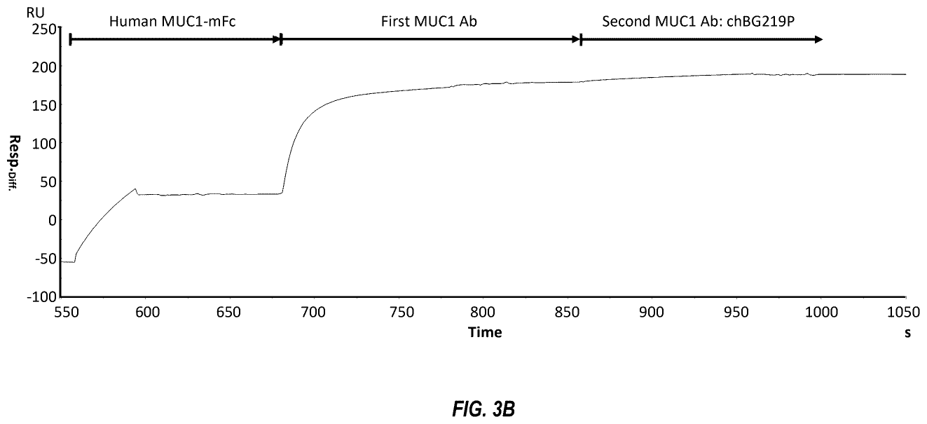

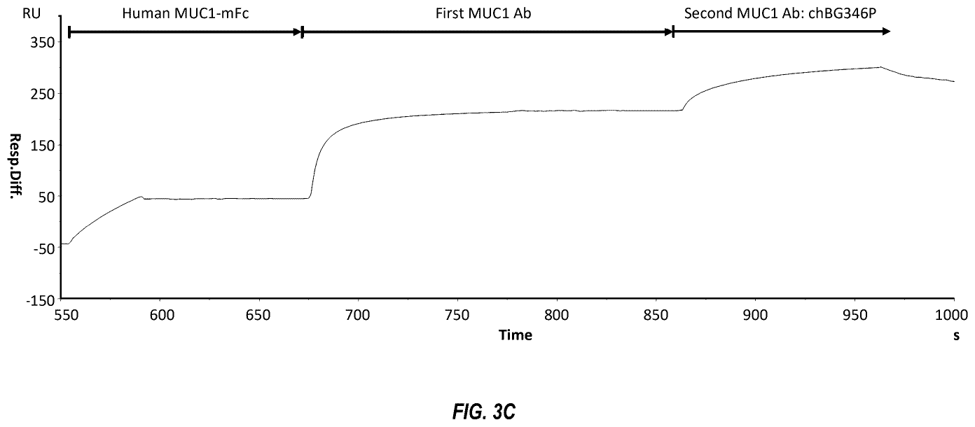

The epitope binning of chimeric anti-MUC1 antibodies was determined by a competitive SPR assay. Briefly, anti-mouse IgG Fc antibody was immobilized on an activated CM5 biosensor chip. Purified huMUC1-SEA-mIgG2a (human MUC1 linked with mouse IgG2a Fc) antigen was flowed over the chip surface and captured by anti-mouse IgG antibody. The reference MUC1-SEA Ab 5F3 (Cancer Immunol Immunother. 2020 July; 69(7):1337-1352) was first injected at saturating antigen binding conditions, followed by injection of chBG138P (FIG. 3A), chBG219P (FIG. 3B), or chBG346P (FIG. 3C). The sensorgrams for epitope binning are shown in FIGS. 3A-3C. As shown in Table 5 below, the three chimeric MUC1 antibodies were grouped into two epitope bins in the MUC1-SEA domain. ChBG138P and chBG219P bind to the same epitope on MUC1-SEA, as does 5F3, which is a different MUC1 epitope from chBG346P. More specifically, chBG138P and chBG219P bind to epitope bin A, while chBG346P binds to bin B.

TABLE 5
Epitope bins of chBG138P, chBG346P, and chBG219P

| Ab # | Bin |
|---|---|
| chBG138P | A |
| chBG346P | B |
| chBG219P | A |

### Example 5. Chimeric Anti-MUC1 Antibodies chBG138P, chBG219P, and chBG346P Show Reduced Interference by Soluble MUC1

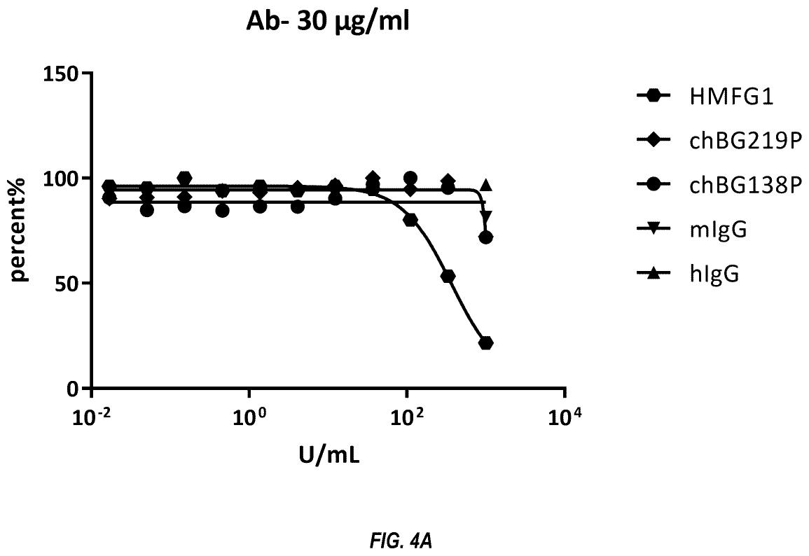

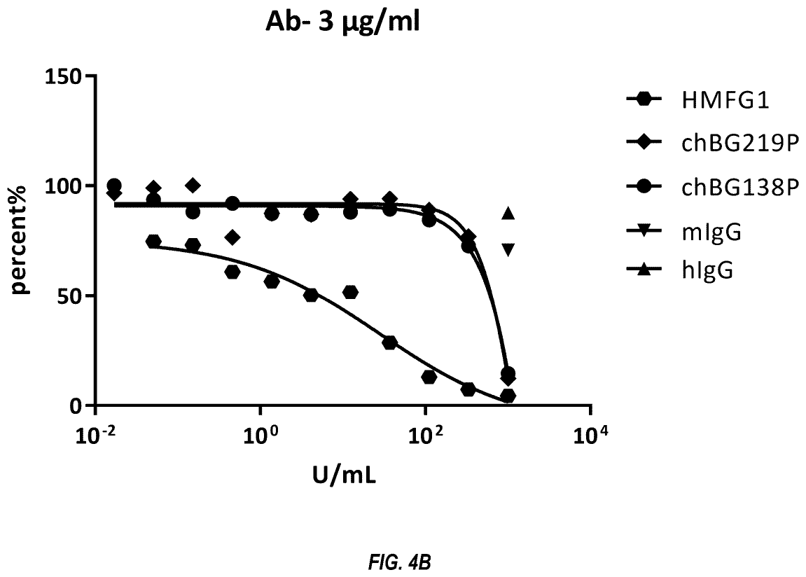

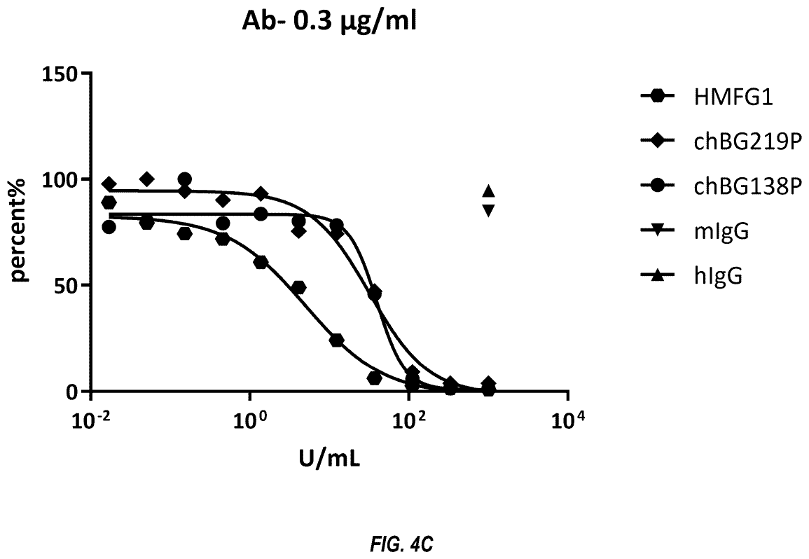

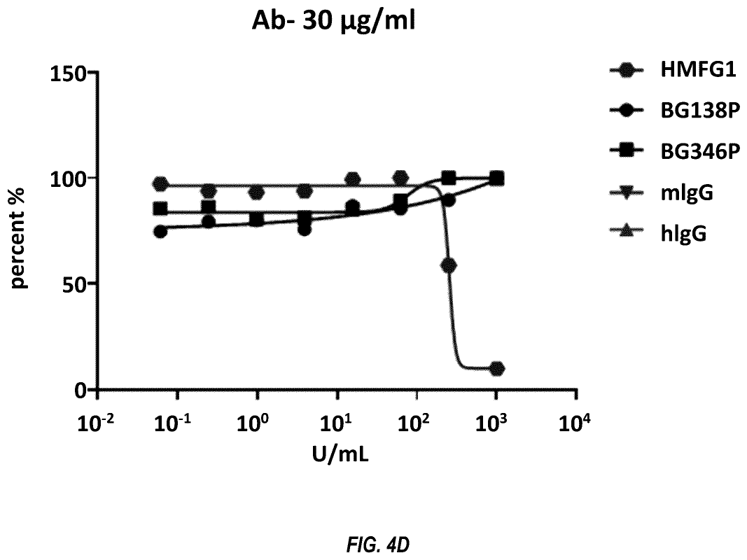

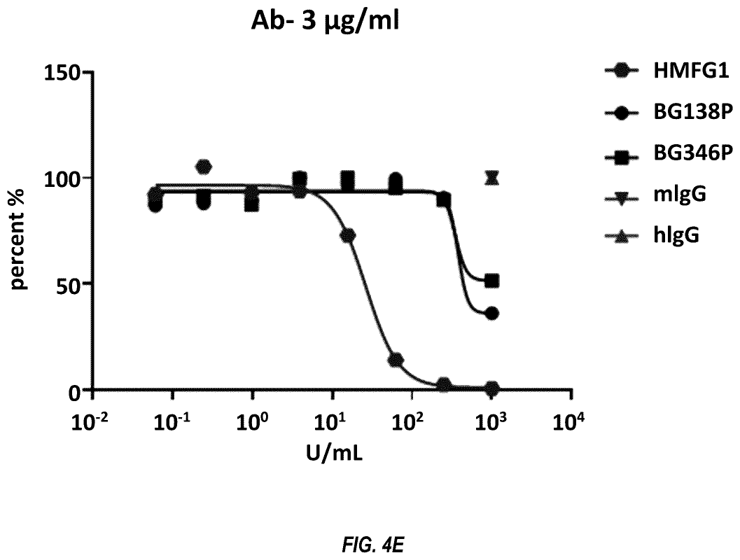

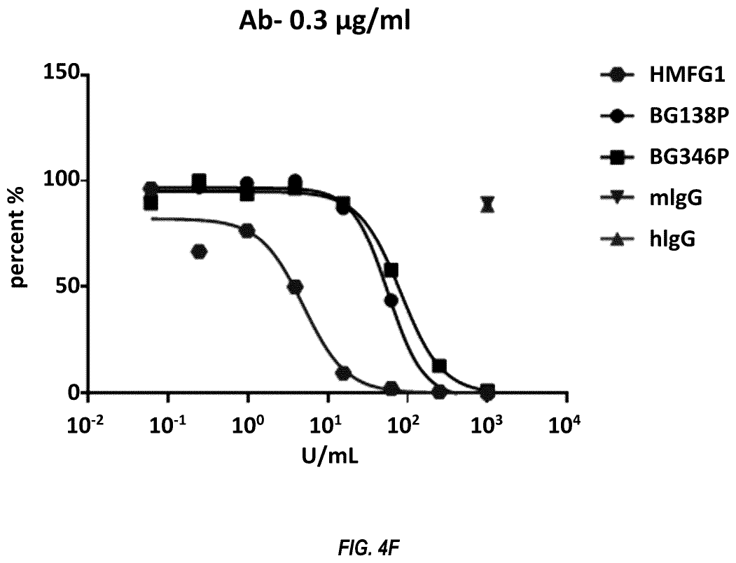

The presence of soluble MUC1 on the specific binding of MUC1 antibodies to MUC1 expressing cells was determined by competitive FACS assay. Briefly, human MUC1 expressing cells were incubated with 30, 3, and 0.3 μg/ml MUC1 antibodies in the presence of serially diluted soluble MUC1 (Shanghai Linc-Bio Science Co. LTD). After washing and incubation with anti-human IgG secondary antibody, fluorescence was measured by flow cytometry. IC₅₀ values for soluble MUC1 blocking MUC1 antibodies binding with MUC1 expressing cells is shown in Table 6, and the blocking curves are shown in FIGS. 4A-4F, in which HMFG1 is a positive control, and mIgG and hIgG1 are negative controls. The profiles indicate that the binding of HMFG to MUC1-expressing cells can be easily interfered with at high, medium, and low antibody concentrations (i.e., 30, 3, 0.3 μg/ml), but chBG138P, chBG219P, and chBG346P binding is only slightly interfered with at the low antibody concentration (i.e., 0.3 μg/ml) (FIGS. 4A-F). Collectively, the profiles in FIG. 4 indicate that the binding of MUC1 antibodies to MUC1-expressing cells showed significantly reduced interference by soluble MUC1 compared to HMFG1 (Abcam), which targets the MUC1 N-terminal.

TABLE 6
IC₅₀ of soluble MUC1 blocking activity
IC₅₀ (U/mL)

| Antibody | 30 μg/ml | 3 μg/ml | 0.3 μg/ml |
|---|---|---|---|
| chBG138P | N.A. | N.A. | 56.8 |
| chBG346P | N.A. | N.A. | 82.1 |
| chBG219P | N.A. | N.A. | 31.8 |

(N.A.: Not available. IC₅₀ data cannot be fitted well and retrieved due to the extremely low interference, as shown from the binding curves in FIGS. 4A, 4B, 4E, and 4F.)

### Example 6. Anti-MUC1 Monoclonal Antibodies Targeting MUC1 Membrane Proximal Region Bind to Cancer Cell Lines but Do Not Bind to Normal T Cells, While MUC1 N-Terminal Targeting Antibodies HMFG1 or 16A can Bind to Normal T Cells

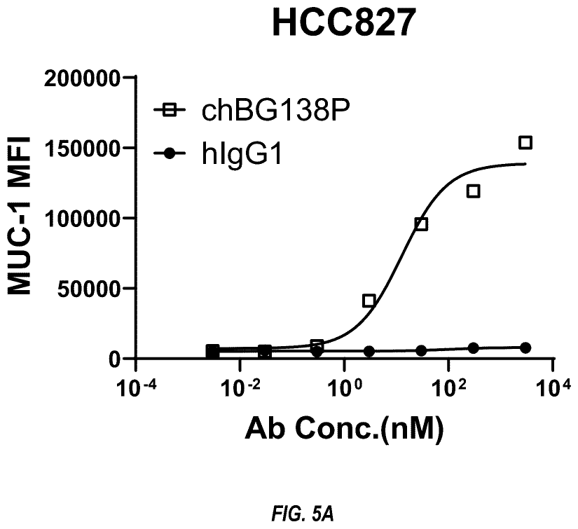

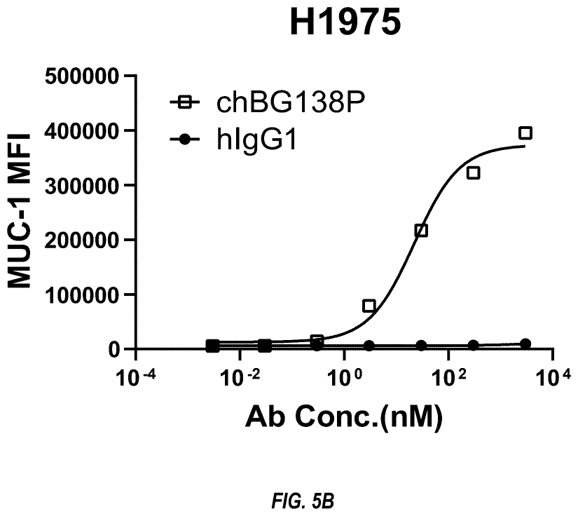

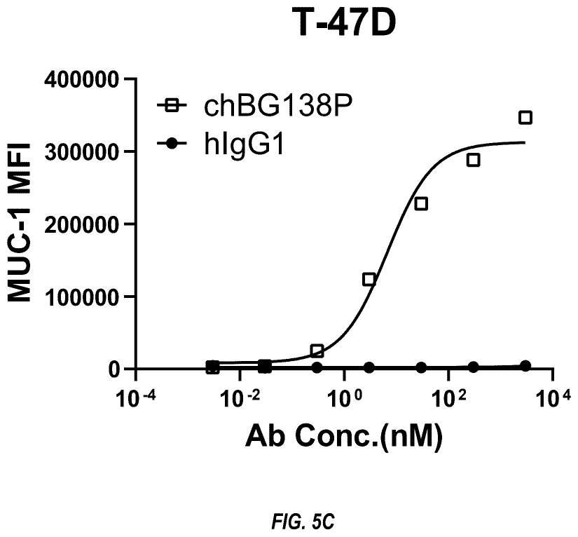

To evaluate whether the anti-MUC1 monoclonal antibodies targeting MUC1 membrane proximal region can differentially bind to MUC1 expressing tumor cells versus MUC1 expressing normal cells, like activated T cells, FACS binding assays were performed. For tumor cell line binding experiments, cells stained with anti-human MUC1 antibody chBG138P or controls (human IgG1) for 1 hour were collected. Then cells were washed twice and followed by staining with a secondary antibody (Alexa Fluor® 647 anti-human IgG Fc) for 30 minutes. Cells were washed and fixed with 1% paraformaldehyde (PFA) in DPBS before FACS analysis. All flow cytometry data were acquired using NovoCyte flow cytometer (ACEA Biosciences, Inc.) and data was analyzed using NovoExpress software. As shown in FIGS. 5A-5C, the chimeric antibody chBG138P, which targets the human MUC1 membrane proximal region, binds to MUC1 expressing tumor cell lines HCC827 (FIG. 5A), H1975 (FIG. 5B), and T-47D (FIG. 5C) in a dose dependent manner (human IgG1 as a negative control), indicating that the MUC1 membrane proximal region targeting antibodies can be used to target cancer cells and thus be applied to treat cancers that express or overexpress MUC1.

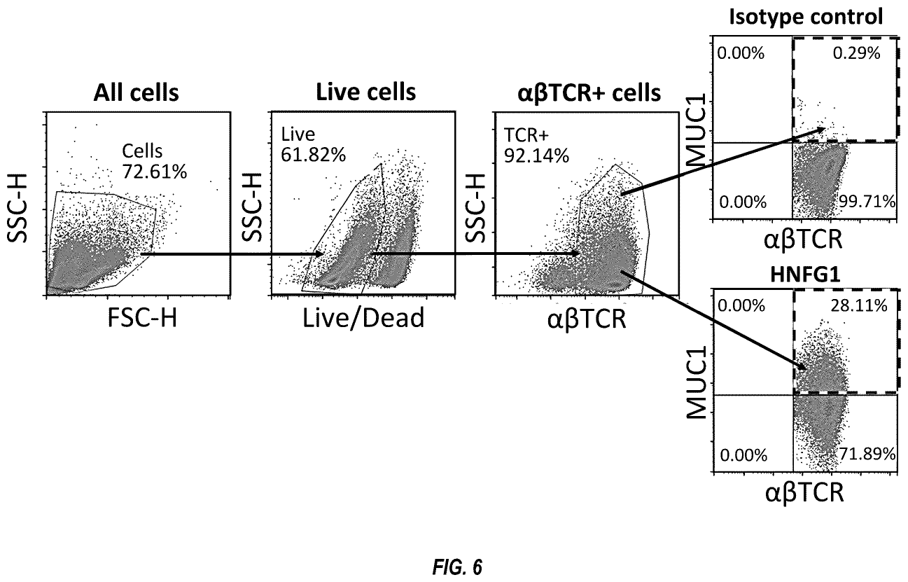

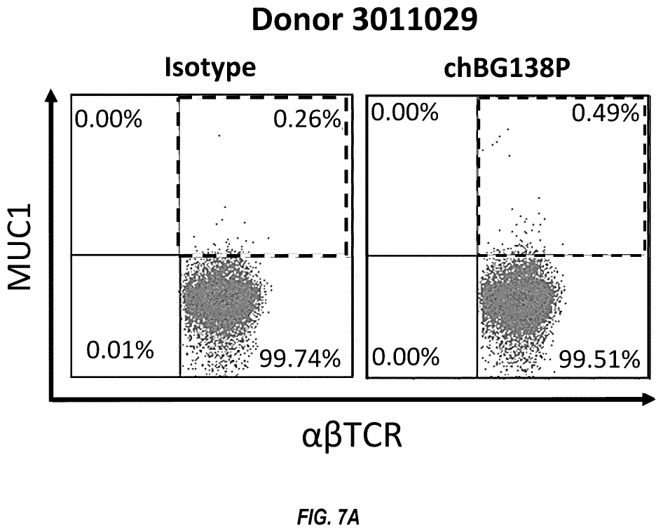

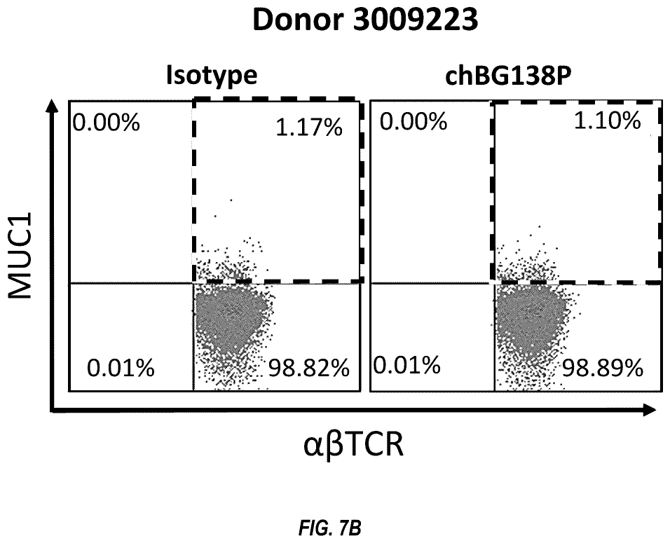

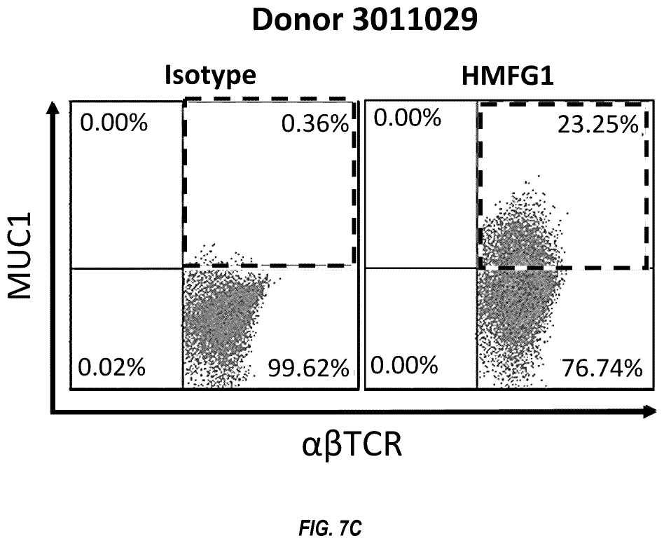

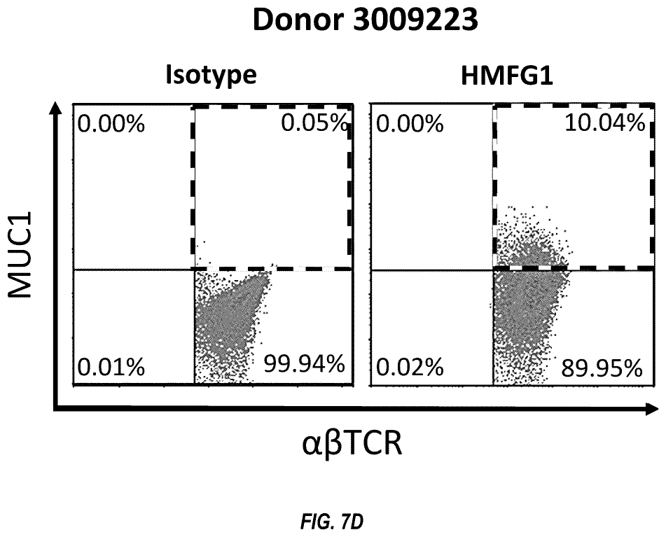

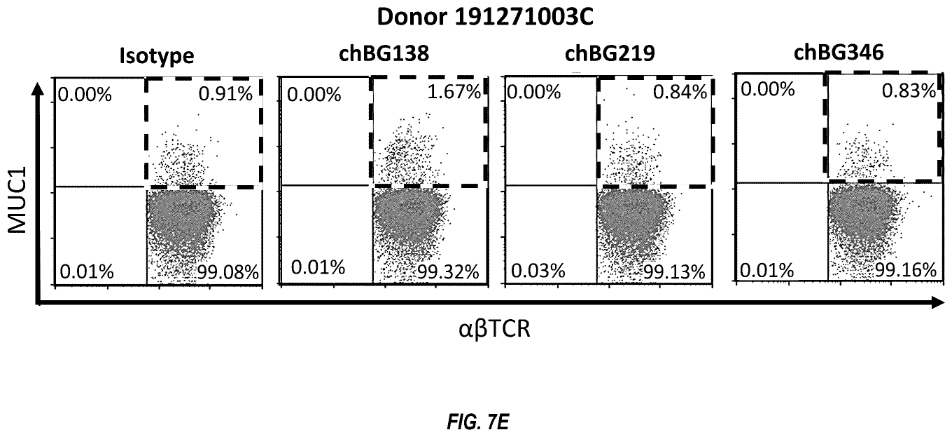

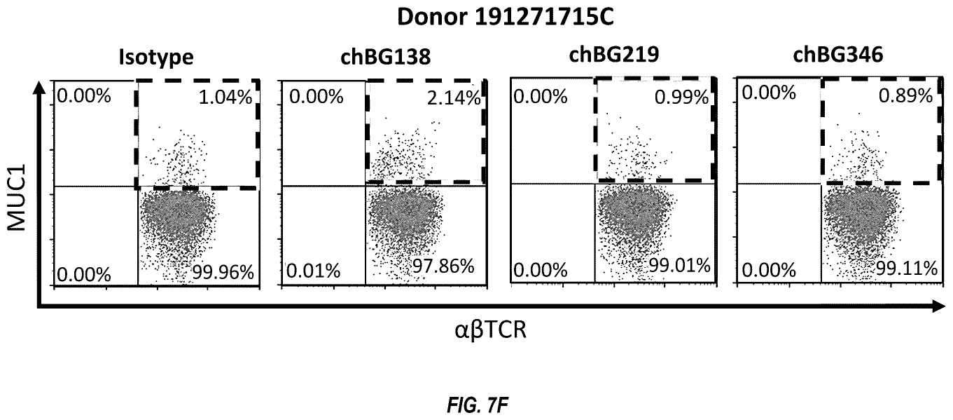

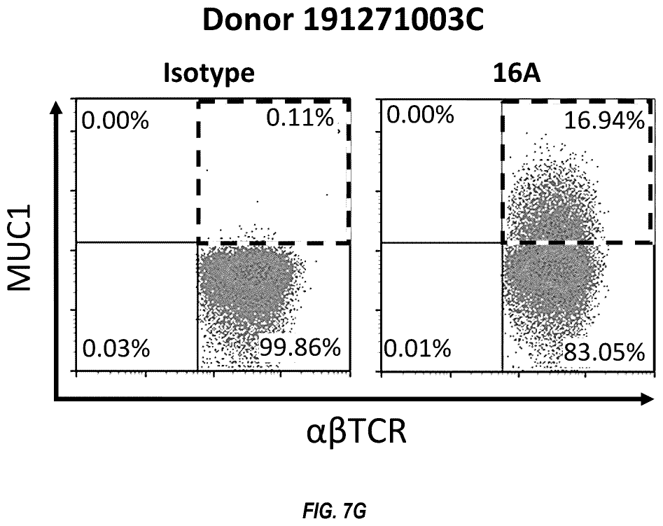

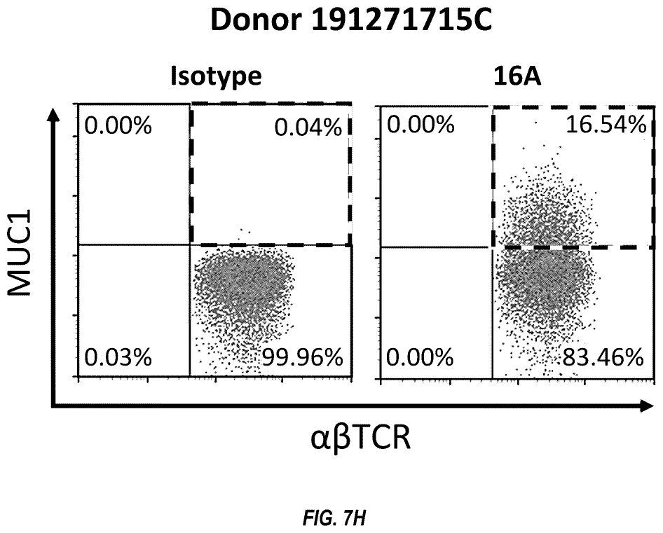

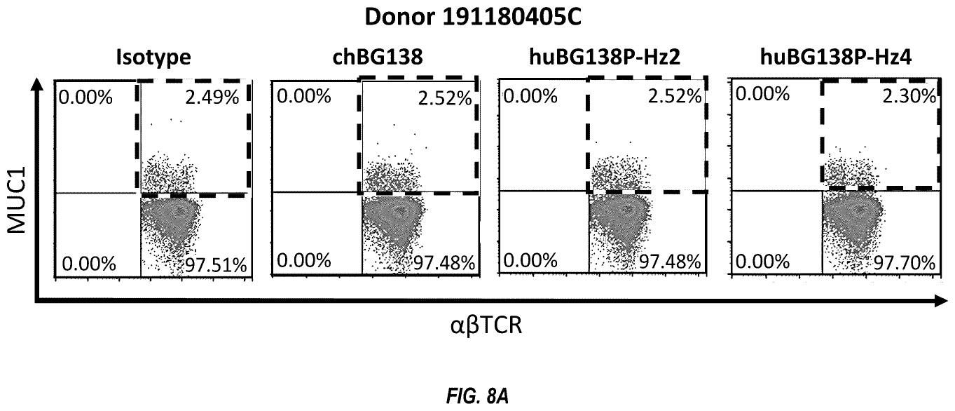

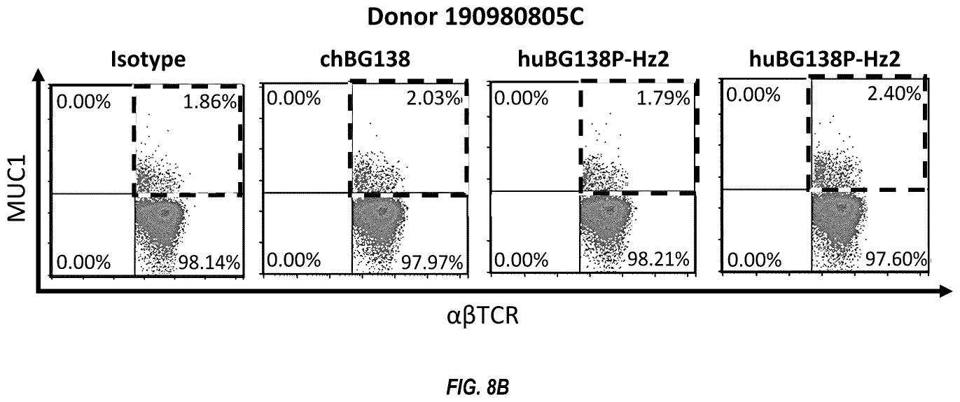

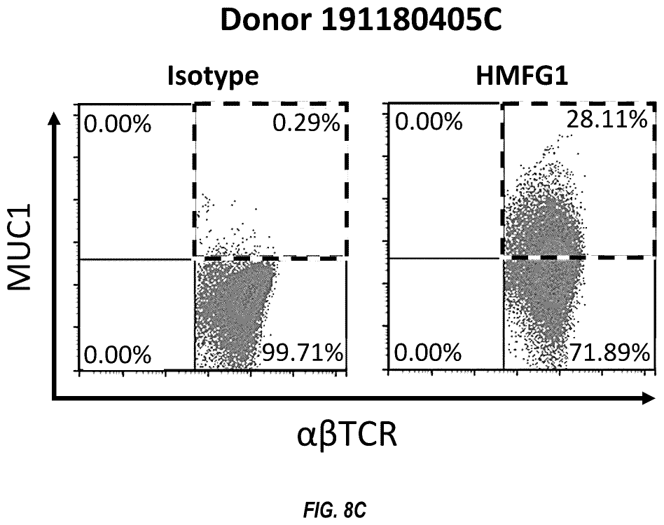

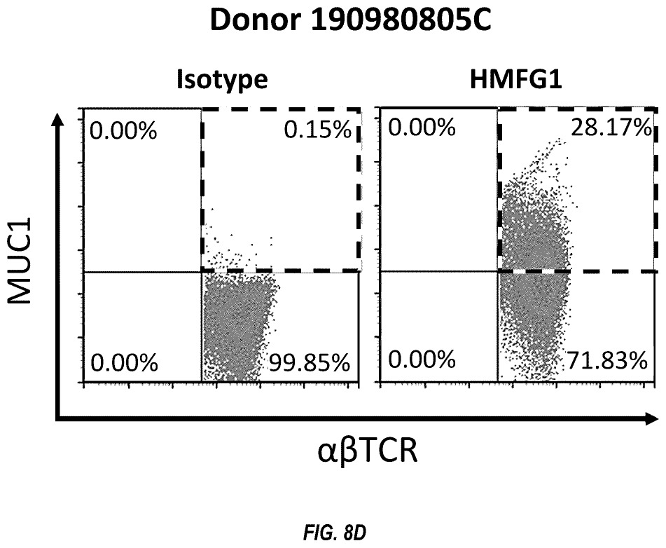

To evaluate whether the monoclonal antibodies targeting MUC1 membrane proximal region can avoid binding to normal cells that express MUC1, an activated T cell binding assay was conducted. Briefly, human peripheral blood mononuclear cells (PBMCs) from six healthy donors purchased from Allcells or Stemcell were stimulated with 1 μg/ml PHA-L for 3 days. Then FACS staining was performed using the stimulated PBMCs. Cell suspensions were pre-incubated with LIVE/DEAD™ Fixable Dead Cell Stain Kit (Invitrogen, REF. #L34964) and Fc receptor blocking solution (100 μg/mL human IgG in FACS buffer), before staining with anti-human antibodies. Cells were washed twice and incubated with 10 μg/mL anti-MUC1 monoclonal antibodies targeting MUC1 membrane proximal region or MUC1 N-terminal targeting antibodies HMFG1 (as positive control, Abcam, cat. #ab215670) or 16A (as positive control, Biolegend, cat. #355608) for 1 hour. Then cells were washed and stained with PE-CY7 anti-human αβ TCR (eBioscience, cat. #25-9986-42), AF647 anti-human IgG Fc (Biolegend, REF. #409320) for 30 minutes. Cells were washed and fixed with 1% PFA in DPBS before FACS analysis. All flow cytometry data were acquired using NovoCyte flow cytometer (ACEA Biosciences, Inc.) and data was analyzed using NovoExpress software. As shown in FIGS. 6 and 7A-7H, none of the chimeric antibodies chBG138P (FIGS. 7A, 7B, 7E, and 7F), chBG219P (FIGS. 7E and 7F), or chBG346P (FIGS. 7E and 7F) binds to normal activated human T cells that express MUC1. However, the MUC1-N terminal targeting antibodies HMFG1 (FIGS. 7C and 7D) and 16A (FIGS. 7G and 7H) bind to a significant portion of activated T cells. FIGS. 8A-8D show that the chimeric (chBG138P) and humanized (huBG138P-Hz2 and huBG138P-Hz4) versions of the antibody BG138P retain similar binding properties with BG138P with no binding to normal activated human T cells that express MUC1 (FIGS. 8A and 8B), while MUC1-N terminal targeting antibody HMFG1 (FIGS. 8C and 8D) binds to normal activated T cells.

The results indicate that, compared to MUC1-N terminal targeting antibodies that bind to both cancer cells and normal T cells, antibodies targeting MUC1 membrane proximal region specifically target cancer cells while sparing normal T cells, and thus may confer an optimized safety profile when used as an anti-tumor therapy in humans.

### Example 7. ChBG138P Induced Internalization of MUC1 in Cancer Cell Lines

chBG138P and human IgG were labeled using pHrodo iFL STP ester (amine-reactive dyes, Invitrogen, Ref. #P36013) following the manufacturer's instructions. Cancer cell lines T-47D, HCC827, and H1975 were incubated with pHrodo-labeled chBG138P (final concentration 10 μg/ml) or human IgG (Human Immunoglobulin for Intravenous Injection, from Hualan) as a negative control at 4° C. for 60 minutes while protected from light. Cells were washed twice with DPBS at 4° C. Half of the cells were kept at 4° C. and half were transferred to 37° C. Cells were incubated at 4° C. or 37° C. for 5 hours. 1 μl/well 7-AAD (7-amino-actinomycin D) was added to all groups. Cells were incubated on ice in darkness for 30 minutes. Cells were washed and fixed with 1% paraformaldehyde (PFA) in DPBS before FACS analysis. All flow cytometry data was acquired using NovoCyte flow cytometer (ACEA Biosciences, Inc.) and data was analyzed using NovoExpress software. Percentage of the pHrodo positive cells were calculated according to manufacturer's instructions (Invitrogen, Ref. #P36013).

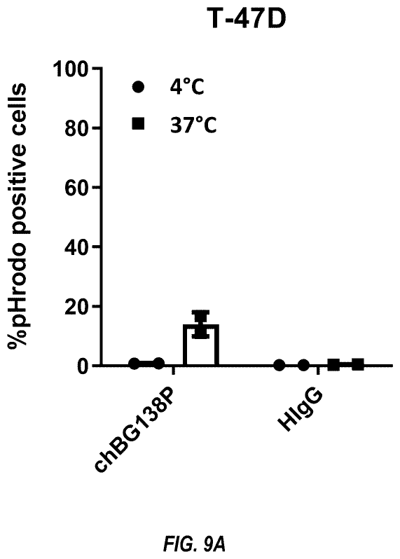

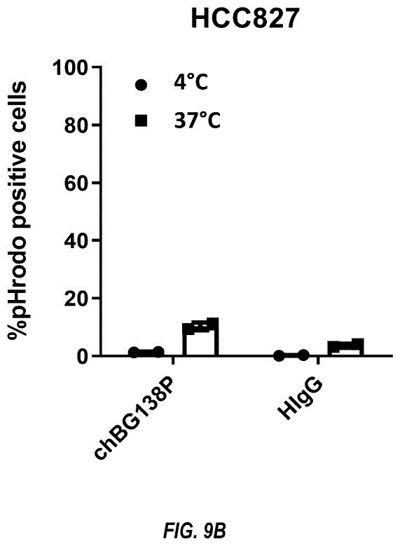

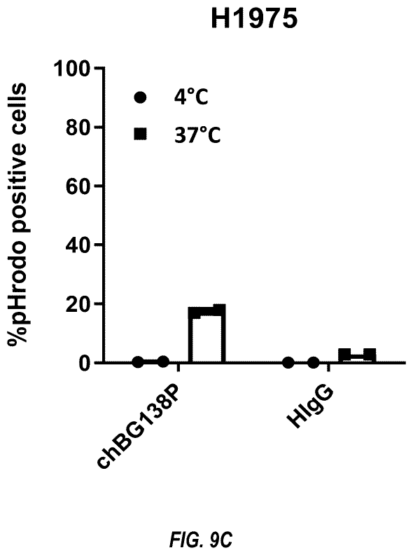

As shown in FIGS. 9A-9C, for all the three cancer cell lines T-47D (9A), HCC827 (9B), and H1975 (9C), around 10-20% of cells were pHrodo positive after incubation for 5 hours at 37° C. The results indicate that MUC1 membrane proximal region targeting antibody chBG138P induced internalization by human cancer-derived cell lines that express MUC1, while the negative control hIgG induced almost no internalization. The internalization property may be beneficial for the design of monoclonal antibody therapy or bi-specific antibody therapy to avoid loss of target on membrane and drug resistance. Additionally, the potential of internalization retains the possibility of the development of antibody-drug conjugates, which include the MUC1 antibodies disclosed herein, for treating MUC1-expressing cancers.

### Example 8. Humanization of the Murine Anti-Human MUC1 Antibody BG138P and BG219P

### Humanization of BG138P

For humanization of murine anti-human MUC1 BG138P antibody, a CDR grafting strategy was employed. Murine BG138P antibody heavy chain and light chain variable domains sequences (abbreviated as VH and VL, respectively) were aligned with human antibody germline sequences by Ig blast tool and human germline frameworks with the highest homology were selected as receptor frameworks for CDR grafting. To maintain the CDR canonical structures, murine version BG138P antibody structure was modeled by Schrodinger software and used for selection of potential back mutation sites to maintain the binding affinity. Each back mutation site was screened by construction of multiple variants without that mutation to evaluate the necessity of that site to be kept in the humanized version. Then, identified critical back mutation sites were combined to construct a second round of humanized variants for affinity characterization.

Specifically, for BG138P VH sequence, IGHV1-3*01 and J region H4 (JH4) germline sequence was selected as the receptor framework. And for BG138P VL sequence, Vκ1-27*01 and J region κ2 was selected as the receptor framework. Antibody variants of the CDR graft-only version (named with suffix “Hz0”), version with all back mutation sites (named with suffix “Bz0”), and multiple variants with reducing each back mutation site (named with suffix “Bz1”-“Bz16”) were constructed with an in-house IgG1/Cκ eukaryotic expression vector, produced with Expi293™ expression system (Thermofisher Scientific), and purified with MabSelect PrismA™ protein A chromatography resin (Cytiva). The antibody variants list and the corresponding VH/VL sequences for this round of screening are summarized in Table 7 (in which “X” indicates the CDR position for grafting).

TABLE 7
Humanization design for back mutation site screening of BG138P

| Name | SEQ ID NO | Note | Sequence |
|---|---|---|---|
| IGHV1- | SEQ ID NO: 34 | Germline | QVQLVQSGAEVKKPGASVKVSCKASGY |
| 3*01 & JH4 | | sequence | TFTXXXXXWVRQAPGQRLEWMGXXX |
| | | with CDRs | XXXXXXXXXXXXXXRVTITRDTSASTA |
| | | represented | YMELSSLRSEDTAVYYCARXXXXXXXX |
| | | as X | XXXWGQGTLVTVSS |
| BG138P- | SEQ ID NO: 35 | VH (only | QVQLVQSGAEVKKPGASVKVSCKASGY |
| Hz0_VH | | mouse | TFTTYTMHWVRQAPGQRLEWMGYINP |
| | | grafted | GSDYTHYNQKFKDRVTITRDTSASTAY |
| | | CDRs) | MELSSLRSEDTAVYYCARQGNYAAYYF |
| | | | DYWGQGTLVTVSS |
| BG138P- | SEQ ID NO: 36 | VH (all | QVQLVQSGAEVKKPGASVKVSCKASGY |
| Bz0_VH | | back | TFTTYTMHWIKQAPGQGLEWIGYINPGS |
| (all back | | mutations) | DYTHYNQKFKDKATLTADKSASTAYME |
| mutations) | | | LYSLRSEDTAVYYCARQGNYAAYYFDY |
| | | | WGQGTLVTVSS |
| BG138P- | SEQ ID NO: 37 | VH variant | QVQLVQSGAEVKKPGASVKVSCKASGY |
| Bz1_VH | | | TFTTYTMHWVKQAPGQGLEWIGYINPG |
| | | | SDYTHYNQKFKDKATLTADKSASTAYM |
| | | | ELYSLRSEDTAVYYCARQGNYAAYYFD |
| | | | YWGQGTLVTVSS |
| BG138P- | SEQ ID NO: 38 | VH variant | QVQLVQSGAEVKKPGASVKVSCKASGY |
| Bz2_VH | | | TFTTYTMHWIRQAPGQGLEWIGYINPGS |
| | | | DYTHYNQKFKDKATLTADKSASTAYME |
| | | | LYSLRSEDTAVYYCARQGNYAAYYFDY |
| | | | WGQGTLVTVSS |
| BG138P- | SEQ ID NO: 39 | VH variant | QVQLVQSGAEVKKPGASVKVSCKASGY |
| Bz3_VH | | | TFTTYTMHWIKQAPGQRLEWIGYINPGS |
| | | | DYTHYNQKFKDKATLTADKSASTAYME |
| | | | LYSLRSEDTAVYYCARQGNYAAYYFDY |
| | | | WGQGTLVTVSS |
| BG138P- | SEQ ID NO: 40 | VH variant | QVQLVQSGAEVKKPGASVKVSCKASGY |
| Bz4_VH | | | TFTTYTMHWIKQAPGQGLEWMGYINPG |
| | | | SDYTHYNQKFKDKATLTADKSASTAYM |
| | | | ELYSLRSEDTAVYYCARQGNYAAYYFD |
| | | | YWGQGTLVTVSS |
| BG138P- | SEQ ID NO: 41 | VH variant | QVQLVQSGAEVKKPGASVKVSCKASGY |
| Bz5_VH | | | TFTTYTMHWIKQAPGQGLEWIGYINPGS |
| | | | DYTHYNQKFKDRATLTADKSASTAYME |
| | | | LYSLRSEDTAVYYCARQGNYAAYYFDY |
| | | | WGQGTLVTVSS |
| BG138P- | SEQ ID NO: 42 | VH variant | QVQLVQSGAEVKKPGASVKVSCKASGY |
| Bz6_VH | | | TFTTYTMHWIKQAPGQGLEWIGYINPGS |
| | | | DYTHYNQKFKDKVTLTADKSASTAYME |
| | | | LYSLRSEDTAVYYCARQGNYAAYYFDY |
| | | | WGQGTLVTVSS |
| BG138P- | SEQ ID NO: 43 | VH variant | QVQLVQSGAEVKKPGASVKVSCKASGY |
| Bz7_VH | | | TFTTYTMHWIKQAPGQGLEWIGYINPGS |
| | | | DYTHYNQKFKDKATITADKSASTAYME |
| | | | LYSLRSEDTAVYYCARQGNYAAYYFDY |
| | | | WGQGTLVTVSS |
| BG138P- | SEQ ID NO: 44 | VH variant | QVQLVQSGAEVKKPGASVKVSCKASGY |
| Bz8_VH | | | TFTTYTMHWIKQAPGQGLEWIGYINPGS |
| | | | DYTHYNQKFKDKATLTRDKSASTAYME |
| | | | LYSLRSEDTAVYYCARQGNYAAYYFDY |
| | | | WGQGTLVTVSS |
| BG138P- | SEQ ID NO: 45 | VH variant | QVQLVQSGAEVKKPGASVKVSCKASGY |
| Bz9_VH | | | TFTTYTMHWIKQAPGQGLEWIGYINPGS |
| | | | DYTHYNQKFKDKATLTADTSASTAYME |
| | | | LYSLRSEDTAVYYCARQGNYAAYYFDY |
| | | | WGQGTLVTVSS |
| BG138P- | SEQ ID NO: 46 | VH variant | QVQLVQSGAEVKKPGASVKVSCKASGY |
| Bz10_VH | | | TFTTYTMHWIKQAPGQGLEWIGYINPGS |
| | | | DYTHYNQKFKDKATLTADKSASTAYME |
| | | | LSSLRSEDTAVYYCARQGNYAAYYFDY |
| | | | WGQGTLVTVSS |
| Vk1- | SEQ ID NO: 47 | Germline | DIQMTQSPSSLSASVGDRVTITCXXXXX |
| 27*01 & JK2 | | sequence | XXXXXXWYQQKPGKVPKLLIYXXXXX |
| | | with CDRs | XXGVPSRFSGSGSGTDFTLTISSLQPEDV |
| | | represented | ATYYCXXXXXXXXXFGQGTKLEIK |
| | | as X | |
| BG138P- | SEQ ID NO: 48 | Vk variant | DIQMTQSPSSLSASVGDRVTITCRASGNI |
| Hz0_Vk | | | HNFLVWYQQKPGKVPKLLIYNAKTLTD |
| | | | GVPSRFSGSGSGTDFTLTISSLQPEDVAT |
| | | | YYCQNFWNVPFTFGQGTKLEIK |
| BG138P- | SEQ ID NO: 49 | Vk variant | DIQMTQSPSSLSASVGDRVTITCRASGNI |
| Bz0_Vk | | | HNFLVWYQQKPGKSPKLLVYNAKTLTD |
| | | | GVPSRFSGSGSGTQYSLTISSLQPEDVAT |
| | | | YYCQNFWNVPFTFGSGTKLEIK |
| BG138P- | SEQ ID NO: 50 | Vk variant | DIQMTQSPSSLSASVGDRVTITCRASGNI |
| Bz11_Vk | | | HNFLVWYQQKPGKVPKLLVYNAKTLT |
| | | | DGVPSRFSGSGSGTQYSLTISSLQPEDVA |
| | | | TYYCQNFWNVPFTFGSGTKLEIK |
| BG138P- | SEQ ID NO: 51 | Vk variant | DIQMTQSPSSLSASVGDRVTITCRASGNI |
| Bz12_Vk | | | HNFLVWYQQKPGKSPKLLIYNAKTLTD |
| | | | GVPSRFSGSGSGTQYSLTISSLQPEDVAT |
| | | | YYCQNFWNVPFTFGSGTKLEIK |
| BG138P- | SEQ ID NO: 52 | Vk variant | DIQMTQSPSSLSASVGDRVTITCRASGNI |
| Bz13_Vk | | | HNFLVWYQQKPGKSPKLLVYNAKTLT |
| | | | DGVPSRFSGSGSGTDYSLTISSLQPEDVA |
| | | | TYYCQNFWNVPFTFGSGTKLEIK |
| BG138P- | SEQ ID NO: 53 | Vk variant | DIQMTQSPSSLSASVGDRVTITCRASGNI |
| Bz14_Vk | | | HNFLVWYQQKPGKSPKLLVYNAKTLT |
| | | | DGVPSRFSGSGSGTQFSLTISSLQPEDVA |
| | | | TYYCQNFWNVPFTFGSGTKLEIK |
| BG138P- | SEQ ID NO: 54 | Vk variant | DIQMTQSPSSLSASVGDRVTITCRASGNI |
| Bz15_Vk | | | HNFLVWYQQKPGKSPKLLVYNAKTLT |
| | | | DGVPSRFSGSGSGTQYTLTISSLQPEDVA |
| | | | TYYCQNFWNVPFTFGSGTKLEIK |
| BG138P- | SEQ ID NO: 55 | Vk variant | DIQMTQSPSSLSASVGDRVTITCRASGNI |
| Bz16_Vk | | | HNFLVWYQQKPGKSPKLLVYNAKTLT |
| | | | DGVPSRFSGSGSGTQYSLTISSLQPEDVA |
| | | | TYYCQNFWNVPFTFGQGTKLEIK |

All generated antibody variants were characterized by SPR affinity to human and cynomolgus MUC1 SEA domain protein (SEQ ID NO: 2) using a Biacore 2000 (Cytiva) system. Variants with larger than 2-fold affinity loss compared to chimeric BG138P (calculated by Kd on single concentration binding kinetics) were selected as Tier 1 back mutation sites, and variants with 1.5-2 fold affinity loss were selected as Tier 2 back mutation sites. The SPR binding affinity data are summarized in Table 8 and Table 9.

TABLE 8
SPR binding affinity of BG138P humanization
variants to human MUC1 SEA protein

| Injection | | | | | | |
|---|---|---|---|---|---|---|
| Variables | | | | 1:1 | | |
| Capture 1 | VH | VL | Analyte 1 | binding | | KD |
| Solution | Sequence | Sequence | Solution | ka | kd | (M) |
| BG138P- | BG138P | BG138P | huMUC1 | 5.26E+05 | 2.26E−04 | 4.30E−10 |
| Chimeric | VH | Vk | SEA | | | |
| BG138P- | Bz0_VH | Bz0_Vk | huMUC1 | 5.49E+05 | 2.55E−04 | 4.64E−10 |
| Bz0 | | | SEA | | | |
| BG138P- | Hz0_VH | Hz0_Vk | huMUC1 | 9.82E+05 | 5.18E−04 | 5.27E−10 |
| Hz0 | | | SEA | | | |
| BG138P- | Bz1_VH | Bz0_Vk | huMUC1 | 5.45E+05 | 2.74E−04 | 5.03E−10 |
| Bz1 | | | SEA | | | |
| BG138P- | Bz2_VH | Bz0_Vk | huMUC1 | 5.74E+05 | 2.60E−04 | 4.53E−10 |
| Bz2 | | | SEA | | | |
| BG138P- | Bz3_VH | Bz0_Vk | huMUC1 | 5.47E+05 | 2.91E−04 | 5.32E−10 |
| Bz3 | | | SEA | | | |
| BG138P- | Bz4_VH | Bz0_Vk | huMUC1 | 5.44E+05 | 2.65E−04 | 4.88E−10 |
| Bz4 | | | SEA | | | |
| BG138P- | Bz5_VH | Bz0_Vk | huMUC1 | 5.61E+05 | 2.67E−04 | 4.75E−10 |
| Bz5 | | | SEA | | | |
| BG138P- | Bz6_VH | Bz0_Vk | huMUC1 | 5.73E+05 | 2.97E−04 | 5.18E−10 |
| Bz6 | | | SEA | | | |
| BG138P- | Bz7_VH | Bz0_Vk | huMUC1 | 5.64E+05 | 2.29E−04 | 4.07E−10 |
| Bz7 | | | SEA | | | |
| BG138P- | Bz8_VH | Bz0_Vk | huMUC1 | 5.00E+05 | 3.04E−04 | 6.08E−10 |
| Bz8 | | | SEA | | | |
| BG138P- | Bz9_VH | Bz0_Vk | huMUC1 | 5.80E+05 | 2.76E−04 | 4.76E−10 |
| Bz9 | | | SEA | | | |
| BG138P- | Bz10_VH | Bz0_Vk | huMUC1 | 5.86E+05 | 2.62E−04 | 4.48E−10 |
| Bz10 | | | SEA | | | |
| BG138P- | Bz0_VH | Bz11_Vk | huMUC1 | 5.59E+05 | 2.87E−04 | 5.13E−10 |
| Bz11 | | | SEA | | | |
| BG138P- | Bz0_VH | Bz12_Vk | huMUC1 | 5.85E+05 | 2.93E−04 | 5.01E−10 |
| Bz12 | | | SEA | | | |
| BG138P- | Bz0_VH | Bz13_Vk | huMUC1 | 5.74E+05 | 2.60E−04 | 4.53E−10 |
| Bz13 | | | SEA | | | |
| BG138P- | Bz0_VH | Bz14_Vk | huMUC1 | 9.35E+05 | 5.69E−04 | 6.08E−10 |
| Bz14 | | | SEA | | | |
| BG138P- | Bz0_VH | Bz15_Vk | huMUC1 | 5.91E+05 | 2.91E−04 | 4.93E−10 |
| Bz15 | | | SEA | | | |
| BG138P- | Bz0_VH | Bz16_Vk | huMUC1 | 5.78E+05 | 2.69E−04 | 4.66E−10 |
| Bz16 | | | SEA | | | |

TABLE 9
SPR binding affinity of BG138P humanization
variants to cyno MUC1 SEA protein

| Injection | | | | | | |
|---|---|---|---|---|---|---|
| Variables | | | | 1:1 | | |
| Capture 1 | VH | VL | Analyte 1 | binding | | KD |
| Solution | Sequence | Sequence | Solution | ka | kd | (M) |
| BG138P- | BG138P | BG138P | cynoMUC1 | 1.07E+06 | 7.12E−03 | 6.63E−09 |
| chimeric | VH | Vk | SEA | | | |
| BG138P- | Bz0_VH | Bz0_Vk | cynoMUC1 | 1.34E+06 | 8.86E−03 | 6.59E−09 |
| Bz0 | | | SEA | | | |
| BG138P- | Hz0_VH | Hz0_Vk | cynoMUC1 | 8.75E+05 | 8.03E−02 | 9.17E−08 |
| Hz0 | | | SEA | | | |
| BG138P- | Bz1_VH | Bz0_Vk | cynoMUC1 | 1.48E+06 | 1.06E−02 | 7.18E−09 |
| Bz1 | | | SEA | | | |
| BG138P- | Bz2_VH | Bz0_Vk | cynoMUC1 | 1.42E+06 | 9.87E−03 | 6.96E−09 |
| Bz2 | | | SEA | | | |
| BG138P- | Bz3_VH | Bz0_Vk | cynoMUC1 | 1.32E+06 | 1.19E−02 | 9.04E−09 |
| Bz3 | | | SEA | | | |
| BG138P- | Bz4_VH | Bz0_Vk | cynoMUC1 | 1.33E+06 | 8.98E−03 | 6.73E−09 |
| Bz4 | | | SEA | | | |
| BG138P- | Bz5_VH | Bz0_Vk | cynoMUC1 | 1.36E+06 | 8.59E−03 | 6.31E−09 |
| Bz5 | | | SEA | | | |
| BG138P- | Bz6_VH | Bz0_Vk | cynoMUC1 | 1.39E+06 | 9.45E−03 | 6.79E−09 |
| Bz6 | | | SEA | | | |
| BG138P- | Bz7_VH | Bz0_Vk | cynoMUC1 | 1.20E+06 | 5.22E−03 | 4.35E−09 |
| Bz7 | | | SEA | | | |
| BG138P- | Bz8_VH | Bz0_Vk | cynoMUC1 | 1.36E+06 | 1.83E−02 | 1.35E−08 |
| Bz8 | | | SEA | | | |
| BG138P- | Bz9_VH | Bz0_Vk | cynoMUC1 | 1.69E+06 | 1.15E−02 | 6.81E−09 |
| Bz9 | | | SEA | | | |
| BG138P- | Bz10_VH | Bz0_Vk | cynoMUC1 | 1.40E+06 | 8.80E−03 | 6.28E−09 |
| Bz10 | | | SEA | | | |
| BG138P- | Bz0_VH | Bz11_Vk | cynoMUC1 | 1.31E+06 | 1.23E−02 | 9.42E−09 |
| Bz11 | | | SEA | | | |
| BG138P- | Bz0_VH | Bz12_Vk | cynoMUC1 | 1.38E+06 | 9.05E−03 | 6.56E−09 |
| Bz12 | | | SEA | | | |
| BG138P- | Bz0_VH | Bz13_Vk | cynoMUC1 | 1.70E+06 | 1.14E−02 | 6.70E−09 |
| Bz13 | | | SEA | | | |
| BG138P- | Bz0_VH | Bz14_Vk | cynoMUC1 | 1.66E+06 | 4.07E−02 | 2.44E−08 |
| Bz14 | | | SEA | | | |
| BG138P- | Bz0_VH | Bz15_Vk | cynoMUC1 | 1.39E+06 | 8.48E−03 | 6.11E−09 |
| Bz15 | | | SEA | | | |
| BG138P- | Bz0_VH | Bz16_Vk | cynoMUC1 | 1.31E+06 | 7.37E−03 | 5.60E−09 |
| Bz16 | | | SEA | | | |

Motif analysis showed amino acids “DG” in the boundary of CDRL2 and FR3 (Kabat No. 56-57) is an aspartic acid isomerization motif and several mutation variants were constructed to remove this post-translational modification (PTM) risk. Four mutations (DG to EG, AG, TG, SG, which variants are named as BG138P-PTM-m1˜m4 in Table 10 and Table 11) were designed and introduced into chimeric BG138P to remove this isomerization motif. SPR binding characterization to human and cynomolgus MUC1 SEA protein were carried out as above, and results are shown in Table 10 and Table 11. All variants showed binding comparable to chimeric BG138P antibody. The mutation m4 (DG to SG) was finally selected for use in a humanized BG138P antibody.

TABLE 10
SPR binding affinity of BG138P PTM removal
variants to human MUC1 SEA protein

| Injection Variables | Analyte 1 | 1:1 | | KD |
|---|---|---|---|---|
| Capture 1 Solution | Solution | binding ka | kd | (M) |
| BG138P-chimeric | huMUC1 SEA | 5.26E+05 | 2.26E−04 | 4.30E−10 |
| BG138P-PTM-m1 | huMUC1 SEA | 5.49E+05 | 2.10E−04 | 3.82E−10 |
| BG138P-PTM-m2 | huMUC1 SEA | 5.26E+05 | 2.07E−04 | 3.95E−10 |
| BG138P-PTM-m3 | huMUC1 SEA | 5.48E+05 | 2.18E−04 | 3.97E−10 |
| BG138P-PTM-m4 | huMUC1 SEA | 5.13E+05 | 2.07E−04 | 4.04E−10 |

TABLE 11
SPR binding affinity of BG138P PTM removal
variants to cyno MUC1 SEA protein

| Injection Variables | Analyte 1 | 1:1 | | KD |
|---|---|---|---|---|
| Capture 1 Solution | Solution | binding ka | kd | (M) |
| BG138P-chimeric | cynoMUC1 | 1.07E+06 | 7.12E−03 | 6.63E−09 |
| | SEA | | | |
| BG138P-PTM-m1 | cynoMUC1 | 1.19E+06 | 7.51E−03 | 6.32E−09 |
| | SEA | | | |
| BG138P-PTM-m2 | cynoMUC1 | 1.02E+06 | 6.99E−03 | 6.85E−09 |
| | SEA | | | |
| BG138P-PTM-m3 | cynoMUC1 | 9.47E+05 | 6.62E−03 | 6.99E−09 |
| | SEA | | | |
| BG138P-PTM-m4 | cynoMUC1 | 9.64E+05 | 6.50E−03 | 6.75E−09 |
| | SEA | | | |

Tier 1 and Tier 2 back mutation sites of VH and VL were combined for the construction of a secondary round of humanization variants, all of which included the “DG” to “SG” PTM removal mutation. The final combination humanized variants (referred as huBG138P-Hz1˜Hz4, or abbreviated as Hz1˜Hz4) are summarized in Table 12, with sequences listed in Table 14.

TABLE 12
Hz1-Hz4 with combination of Tier 1 and Tier 2 back mutation sites

| | Hz8_VH (R71A) | Hz3, 8, 9_VH (R44G, |
|---|---|---|
| | (Tier 1) | R71A, T73K) (Tier 2) |
| VH/VL mutations | (SEQ ID NO: 57) | (SEQ ID NO: 58) |
| Hz14_Vk (F71Y) (Tier 1) | huBG138P-Hz1 | huBG138P-Hz3 |
| (SEQ ID NO: 59) | | |
| Hz11, 13, 14_Vk (V43S, | huBG138P-Hz2 | huBG138P-Hz4 |
| D70Q, F71Y) (Tier 2) | | |
| (SEQ ID NO: 60) | | |

Then an SPR assay was performed to evaluate the binding affinity of huBG138P-Hz1˜Hz4 to human and cyno MUC1 SEA. The SPR assay showed that all variants maintained comparable binding as chimeric BG138P, with data summarized in Table 13.

TABLE 13
SPR binding affinity of BG138P humanized variants
Hz1~Hz4 to human and cyno MUC1 SEA protein

| Injection | | | | |
|---|---|---|---|---|
| Variables Capture | Analyte 1 | | | KD |
| 1 Solution | Solution | ka | kd | (M) |
| BG138P-chimeric | huMUC1 SEA | 7.03E+05 | 1.39E−04 | 1.97E−10 |
| huBG138P-Hz1 | huMUC1 SEA | 6.14E+05 | 1.48E−04 | 2.41E−10 |
| huBG138P-Hz2 | huMUC1 SEA | 9.67E+05 | 2.18E−04 | 2.25E−10 |
| huBG138P-Hz3 | huMUC1 SEA | 7.19E+05 | 1.74E−04 | 2.42E−10 |
| huBG138P-Hz4 | huMUC1 SEA | 9.59E+05 | 2.13E−04 | 2.23E−10 |
| BG138P-chimeric | cynoMUC1 SEA | 1.26E+06 | 7.09E−03 | 5.64E−09 |
| huBG138P-Hz1 | cynoMUC1 SEA | 2.58E+06 | 1.97E−02 | 7.64E−09 |
| huBG138P-Hz2 | cynoMUC1 SEA | 1.52E+06 | 1.01E−02 | 6.67E−09 |
| huBG138P-Hz3 | cynoMUC1 SEA | 1.89E+06 | 1.59E−02 | 8.42E−09 |
| huBG138P-Hz4 | cynoMUC1 SEA | 1.47E+06 | 7.26E−03 | 4.94E−09 |

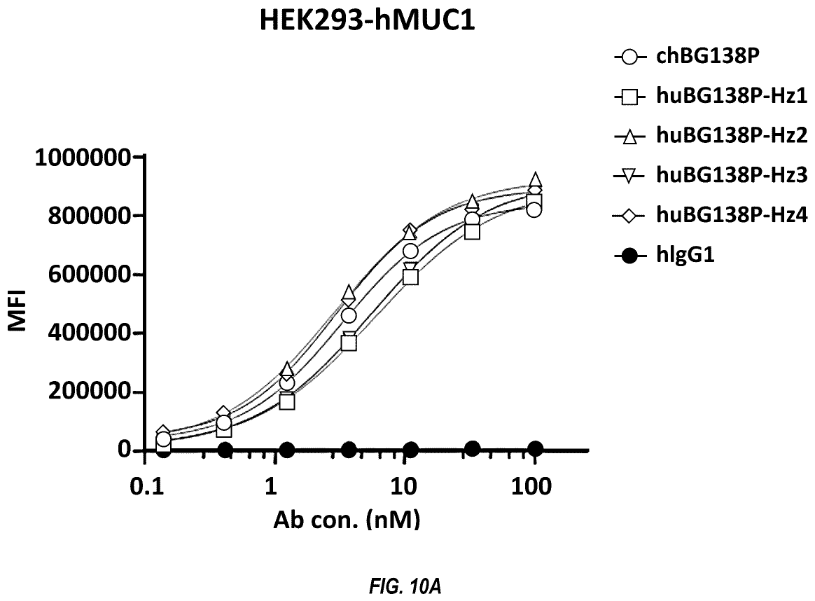

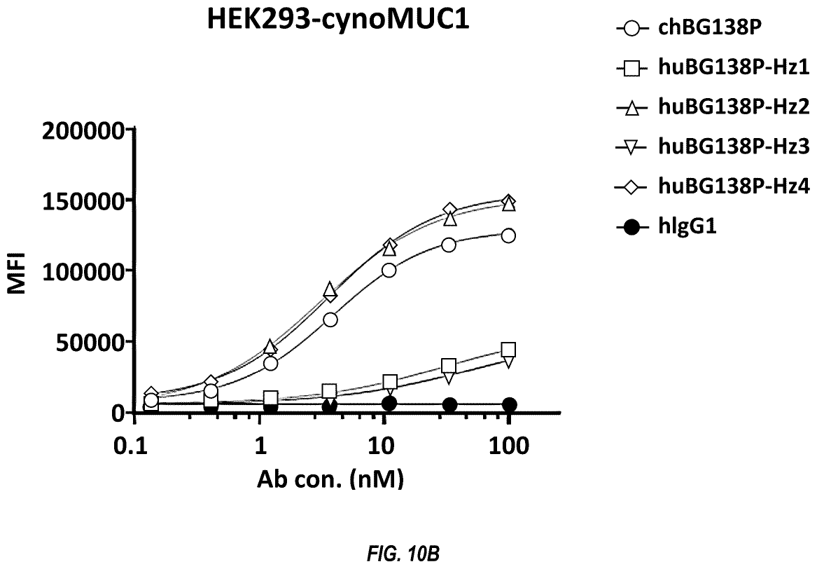

Then a FACS binding (iQue3™ FACS, Sartorius) assay using HEK293-hMUC1 and HEK293-cynoMUC1 was carried out to determine the cell-based binding activity of Hz1˜Hz4. The FACS binding data of chimeric BG138P and its humanized antibodies huBG138P-Hz1˜Hz4, with hIgG1 as control, was processed by GraphPad Prism software and is shown in FIGS. 10A and 10B. Results indicate BG138P-Hz2 and BG138P-Hz4 keep the cell-based binding activity to both human and cyno MUC1 similar to that of chimeric BG138P, and BG138P-Hz1 and BG138P-Hz3 keep similar cell-based binding activity to human MUC1 as that of chimeric BG138P but lower cell-based binding activity to cyno MUC1 than that of chimeric BG138P.

HCDRs, LCDRs, VH and VL amino acid sequences of huBG138P-Hz2 are provided in Table 14.

TABLE 14
Sequence table of humanized variants of BG138P

| Antibody | SEQ ID NO | Note | Sequence |
|---|---|---|---|
| huBG138P- | SEQ ID NO: 4 | HCDR1 | TYTMH |
| Hz2 | | (Kabat) | |
| | SEQ ID NO: 5 | HCDR2 | YINPGSDYTHYNQKFKD |
| | | (Kabat) | |
| | SEQ ID NO: 6 | HCDR3 | QGNYAAYYFDY |
| | | (Kabat) | |
| | SEQ ID NO: 7 | LCDR1 | RASGNIHNFLV |
| | | (Kabat) | |
| | SEQ ID NO: 56 | LCDR2 | NAKTLTS |
| | | (Kabat) | |
| | SEQ ID NO: 9 | LCDR3 | QNFWNVPFT |
| | | (Kabat) | |
| huBG138P- | SEQ ID NO: 57 | VH AA | QVQLVQSGAEVKKPGASVKVSCKASG |
| Hz1 | | | YTFTTYTMHWVRQAPGQRLEWMGYI |
| huBG138P- | | | NPGSDYTHYNQKFKDRVTITADTSAST |
| Hz 2 | | | AYMELSSLRSEDTAVYYCARQGNYAAY |
| | | | YFDYWGQGTLVTVSS |
| huBG138P- | SEQ ID NO: 58 | VH AA | QVQLVQSGAEVKKPGASVKVSCKASG |
| Hz3 | | | YTFTTYTMHWVRQAPGQGLEWMGYI |
| huBG138P- | | | NPGSDYTHYNQKFKDRVTITADKSAST |
| Hz 4 | | | AYMELSSLRSEDTAVYYCARQGNYAAY |
| | | | YFDYWGQGTLVTVSS |
| huBG138P- | SEQ ID NO: 59 | Vk AA | DIQMTQSPSSLSASVGDRVTITCRASGN |
| Hz1 | | | IHNFLVWYQQKPGKVPKLLIYNAKTLT |
| huBG138P- | | | SGVPSRFSGSGSGTDYTLTISSLQPEDVA |
| Hz 3 | | | TYYCQNFWNVPFTFGQGTKLEIK |
| huBG138P- | SEQ ID NO: 60 | Vk AA | DIQMTQSPSSLSASVGDRVTITCRASGN |
| Hz2 | | | IHNFLVWYQQKPGKSPKLLIYNAKTLTS |
| huBG138P- | | | GVPSRFSGSGSGTQYTLTISSLQPEDVAT |
| Hz 4 | | | YYCQNFWNVPFTFGQGTKLEIK |

### Humanization of BG219P

For humanization of BG219P, human germline IgG genes were searched for sequences that share high degrees of homology to the protein sequences of BG219P variable regions by sequence comparison against the human immunoglobulin gene database in IMGT. The human IGHV and IGKV genes that are present in human antibody repertoires with high frequencies and highly homologous to murine BG219P were selected as the templates for humanization.

Humanization was carried out by CDR-grafting followed by critical back mutations being incorporated. The humanized antibodies were engineered as human IgG1 wild type format by using an in-house developed expression vector. In the initial round of humanization, mutations from murine to human amino acid residues in framework regions were guided by 3D structure analysis and the murine framework residues with structural importance for maintaining the canonical structural of CDRs being retained in the first round of the humanization design. BG219P-Bz0, which is the antibody variant of CDRs graft version with all back mutation sites, among all the 19 variants generated, is the variant with theoretical binding capacity approximate to parental murine antibody BG219P.

Specifically, BG219P-Bz0 was generated as described herein. Human germline variable gene IGKV1-39*01 and IGKJ2*01 and human germline variable gene IGHV3-23*01 and IGHJ6*01 were selected as receptor frameworks for BG219P VL and VH sequences. LCDRs of murine BG219P were grafted into the framework of human germline variable gene IGKV1-39*01 and IGKJ2*01 with D17E, A43S, 148V, T69P, and F71Y murine framework residues. The amino acid sequence and DNA sequence of the resulting BG219P-Bz0 VL are shown in Table 16. HCDRs of murine BG219P were grafted into the framework of human germline variable gene IGHV3-23*01 and IGHJ6*01 with S30N, S49A, A93T, and K94R murine framework residues retained. The amino acid sequence and DNA sequence of the resulting BG219P-Bz0 VH are shown in Table 16.

Beginning with the humanized BG219P antibody huBG219P-Bz0, several additional amino acid changes in the CDR region of both VH and VL were made to further improve the biophysical properties for therapeutic use in humans. The considerations included removing post-translational modifications and improving heat stability (Tm) while maintaining binding activities.

More than thirty humanized BG219P (also referred as huBG219P) variants were constructed with in-house IgG1/Cκ eukaryotic expression vectors that contain constant regions of a human wildtype IgG1 and kappa chain, respectively, with easy adapting sub-cloning sites. The variants were produced by transient transfection of the plasmids into ExpiCHO-s cells (Thermofisher Scientific). The conditioned medium was harvested and the variants were purified using a MabSelect™ SuRe column (Cytiva) followed by UF/DF to change the buffer. All the purified antibodies were stored in a −80° C. freezer in small aliquots.

For affinity determination, antibodies were captured by anti-human Fc surface, and used in the affinity assay based on surface plasmon resonance (SPR) technology. The results of SPR-determined binding profiles of anti-MUC1 antibodies are summarized in Table 15. huBG219P-E39 and huBG219P-E43 have similar binding affinities with dissociation constant at 35.2 pM and 30.3 pM, respectively, which are comparable to that of chimeric BG219P (39.8 pM). The sequences of huBG219P-E39 and huBG219P-E43 are provided in Table 16.

TABLE 15
Comparison of huBG219P binding affinities
to MUC1 SEA-Fc by SPR

| Antibody | kₒₙ (M−1s−1) | k_{off} (s−1) | K_{D} (nM) |
|---|---|---|---|
| chBG219P | 1.44E+06 | 5.74E−05 | 3.98E−011 |
| huBG219P-E39 | 1.40E+06 | 4.92E−05 | 3.52E−011 |
| huBG219P-E43 | 1.61E+06 | 4.86E−05 | 3.03E−011 |

TABLE 16
Sequence table

| Ab # | SEQ ID NO | Note | Sequence |
|---|---|---|---|
| huBG219P- | SEQ ID NO: 24 | HCDR1 | DYIMS |
| Bz0 | | Kabat) | |
| | SEQ ID NO: 25 | HCDR2 | TVSSGGDNTYYPDSVKG |
| | | (Kabat) | |
| | SEQ ID NO: 26 | HCDR3 | YGGLGAMDY |
| | | (Kabat) | |
| | SEQ ID NO: 27 | LCDR1 | RASGNIHNYLT |
| | | (Kabat) | |
| | SEQ ID NO: 28 | LCDR2 | NAKTLAD |
| | | (Kabat) | |
| | SEQ ID NO: 29 | LCDR3 | QHFWSVPYT |
| | | (Kabat) | |
| | SEQ ID NO: 61 | VH AA | EVQLLESGGGLVQPGGSLRLSCAAS |
| | | | GFTFNDYIMSWVRQAPGKGLEWVA |
| | | | TVSSGGDNTYYPDSVKGRFTISRDNS |
| | | | KNTLYLQMNSLRAEDTAVYYCTRY |
| | | | GGLGAMDYWGQGTLVTVSS |
| | SEQ ID NO: 62 | VL AA | DIQMTQSPSSLSASVGERVTITCRAS |
| | | | GNIHNYLTWYQQKPGKSPKLLVYN |
| | | | AKTLADGVPSRFSGSGSGPDYTLTIS |
| | | | SLQPEDFATYYCQHFWSVPYTFGQG |
| | | | TKLEIK |
| | SEQ ID NO: 63 | VH DNA | GAAGTGCAGCTGCTCGAGAGCGGG |
| | | | GGCGGCCTCGTGCAACCTGGCGGG |
| | | | AGCCTGAGACTGAGCTGCGCCGCT |
| | | | AGCGGCTTCACCTTCAACGACTAC |
| | | | ATCATGAGCTGGGTGAGACAAGCC |
| | | | CCCGGCAAGGGCCTGGAGTGGGTG |
| | | | GCCACCGTGAGCAGCGGGGGCGAC |
| | | | AACACCTACTACCCCGACAGCGTG |
| | | | AAGGGCAGATTCACCATCAGCAGA |
| | | | GACAACAGCAAGAACACCCTGTAC |
| | | | CTGCAGATGAACAGCCTGAGAGCC |
| | | | GAGGACACCGCCGTGTACTACTGC |
| | | | ACAAGATACGGCGGCCTGGGCGCC |
| | | | ATGGACTACTGGGGCCAAGGCACC |
| | | | CTGGTGACCGTCAGCTCC |
| | SEQ ID NO: 64 | VL DNA | GACATTCAGATGACACAGAGCCCT |
| | | | AGCAGCCTGAGCGCTAGCGTGGGC |
| | | | GAGAGAGTGACCATCACCTGCAGA |
| | | | GCTAGCGGCAACATCCACAACTAC |
| | | | CTGACCTGGTATCAGCAGAAGCCC |
| | | | GGCAAGAGCCCCAAGCTGCTGGTG |
| | | | TACAACGCCAAGACCCTGGCCGAC |
| | | | GGCGTGCCTAGCAGATTCAGCGGC |
| | | | AGCGGCAGCGGCCCCGACTACACC |
| | | | CTGACCATCAGCAGCCTGCAGCCC |
| | | | GAGGACTTCGCCACCTACTACTGTC |
| | | | AGCACTTCTGGAGCGTGCCCTACA |
| | | | CCTTCGGCCAAGGCACCAAGCTGG |
| | | | AGATCAAG |
| huBG219P- | SEQ ID NO: 24 | HCDR1 | DYIMS |
| E39 | | (Kabat) | |
| | SEQ ID NO: 25 | HCDR2 | TVSSGGDNTYYPDSVKG |
| | | (Kabat) | |
| | SEQ ID NO: 26 | HCDR3 | YGGLGAMDY |
| | | (Kabat) | |
| | SEQ ID NO: 27 | LCDR1 | RASGNIHNYLT |
| | | (Kabat) | |
| | SEQ ID NO: 65 | LCDR2 | NAKTLAS |
| | | (Kabat) | |
| | SEQ ID NO: 29 | LCDR3 | QHFWSVPYT |
| | | (Kabat) | |
| | SEQ ID NO: 61 | VH | EVQLLESGGGLVQPGGSLRLSCAAS |
| | | AA | GFTFNDYIMSWVRQAPGKGLEWVAT |
| | | | VSSGGDNTYYPDSVKGRFTISRDNSK |
| | | | NTLYLQMNSLRAEDTAVYYCTRYGG |
| | | | LGAMDYWGQGTLVTVSS |
| | SEQ ID NO: 66 | VL | DIQMTQSPSSLSASVGDRVTITCRAS |
| | | AA | GNIHNYLTWYQQKPGKAPKLLIYNA |
| | | | KTLASGVPSRFSGSGSGTDFTLTISSL |
| | | | QPEDFATYYCQHFWSVPYTFGQGTK |
| | | | LEIK |
| | SEQ ID NO: 63 | VH | GAAGTGCAGCTGCTCGAGAGCGGG |
| | | DNA | GGCGGCCTCGTGCAACCTGGCGGG |
| | | | AGCCTGAGACTGAGCTGCGCCGCT |
| | | | AGCGGCTTCACCTTCAACGACTAC |
| | | | ATCATGAGCTGGGTGAGACAAGCC |
| | | | CCCGGCAAGGGCCTGGAGTGGGTG |
| | | | GCCACCGTGAGCAGCGGGGGCGAC |
| | | | AACACCTACTACCCCGACAGCGTG |
| | | | AAGGGCAGATTCACCATCAGCAGA |
| | | | GACAACAGCAAGAACACCCTGTAC |
| | | | CTGCAGATGAACAGCCTGAGAGCC |
| | | | GAGGACACCGCCGTGTACTACTGC |
| | | | ACAAGATACGGCGGCCTGGGCGCC |
| | | | ATGGACTACTGGGGCCAAGGCACC |
| | | | CTGGTGACCGTCAGCTCC |
| | SEQ ID NO: 67 | VL | GACATTCAGATGACACAGAGCCCT |
| | | DNA | AGCAGCCTGAGCGCTAGCGTGGGC |
| | | | GACAGAGTGACCATCACCTGCAGA |
| | | | GCTAGCGGCAACATCCACAACTAC |
| | | | CTGACCTGGTATCAGCAGAAGCCC |
| | | | GGCAAGGCCCCCAAGCTGCTGATC |
| | | | TACAACGCCAAGACCCTGGCCAGC |
| | | | GGCGTGCCTAGCAGATTCAGCGGC |
| | | | AGCGGCAGCGGCACCGACTTCACC |
| | | | CTGACCATCAGCAGCCTGCAGCCC |
| | | | GAGGACTTCGCCACCTACTACTGTC |
| | | | AGCACTTCTGGAGCGTGCCCTACA |
| | | | CCTTCGGCCAAGGCACCAAGCTGG |
| | | | AGATCAAG |
| huBG219P- | SEQ ID NO: 24 | HCDR1 | DYIMS |
| E43 | | (Kabat) | |
| | SEQ ID NO: 25 | HCDR2 | TVSSGGDNTYYPDSVKG |
| | | (Kabat) | |
| | SEQ ID NO: 26 | HCDR3 | YGGLGAMDY |
| | | (Kabat) | |
| | SEQ ID NO: 27 | LCDR1 | RASGNIHNYLT |
| | | (Kabat) | |
| | SEQ ID NO: 65 | LCDR2 | NAKTLAS |
| | | (Kabat) | |
| | SEQ ID NO: 29 | LCDR3 | QHFWSVPYT |
| | | (Kabat) | |
| | SEQ ID NO: 61 | VH | EVQLLESGGGLVQPGGSLRLSCAAS |
| | | AA | GFTFNDYIMSWVRQAPGKGLEWVAT |
| | | | VSSGGDNTYYPDSVKGRFTISRDNSK |
| | | | NTLYLQMNSLRAEDTAVYYCTRYGG |
| | | | LGAMDYWGQGTLVTVSS |
| | SEQ ID NO: 68 | VL | DIQMTQSPSSLSASVGDRVTITCRAS |
| | | AA | GNIHNYLTWYQQKPGKAPKLLIYNA |
| | | | KTLASGVPSRFSGSGSGTDYTLTISSL |
| | | | QPEDFATYYCQHFWSVPYTFGQGTK |
| | | | LEIK |
| | SEQ ID NO: 63 | VH | GAAGTGCAGCTGCTCGAGAGCGGG |
| | | DNA | GGCGGCCTCGTGCAACCTGGCGGG |
| | | | AGCCTGAGACTGAGCTGCGCCGCT |
| | | | AGCGGCTTCACCTTCAACGACTAC |
| | | | ATCATGAGCTGGGTGAGACAAGCC |
| | | | CCCGGCAAGGGCCTGGAGTGGGTG |
| | | | GCCACCGTGAGCAGCGGGGGCGAC |
| | | | AACACCTACTACCCCGACAGCGTG |
| | | | AAGGGCAGATTCACCATCAGCAGA |
| | | | GACAACAGCAAGAACACCCTGTAC |
| | | | CTGCAGATGAACAGCCTGAGAGCC |
| | | | GAGGACACCGCCGTGTACTACTGC |
| | | | ACAAGATACGGCGGCCTGGGCGCC |
| | | | ATGGACTACTGGGGCCAAGGCACC |
| | | | CTGGTGACCGTCAGCTCC |
| | SEQ ID NO: 69 | VL | GACATTCAGATGACACAGAGCCCT |
| | | DNA | AGCAGCCTGAGCGCTAGCGTGGGC |
| | | | GACAGAGTGACCATCACCTGCAGA |
| | | | GCTAGCGGCAACATCCACAACTAC |
| | | | CTGACCTGGTATCAGCAGAAGCCC |
| | | | GGCAAGGCCCCCAAGCTGCTGATC |
| | | | TACAACGCCAAGACCCTGGCCAGC |
| | | | GGCGTGCCTAGCAGATTCAGCGGC |
| | | | AGCGGCAGCGGCACCGACTACACC |
| | | | CTGACCATCAGCAGCCTGCAGCCC |
| | | | GAGGACTTCGCCACCTACTACTGTC |
| | | | AGCACTTCTGGAGCGTGCCCTACA |
| | | | CCTTCGGCCAAGGCACCAAGCTGG |
| | | | AGATCAAG |

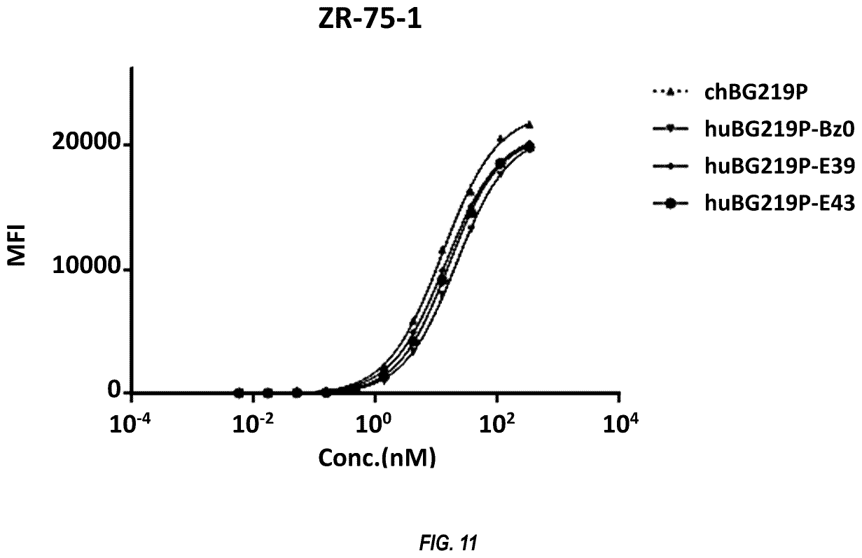

To evaluate the binding activity of anti-MUC1 antibodies to bind native MUC1 on live cells, ZR-75 cells were used for FACS-based binding assay. Live ZR-75 cells were seeded in 96-well plate and were incubated with a series of dilutions of chimeric or humanized BG219Ps. Goat anti-human IgG was used as a second antibody to detect antibody binding to the cell surface. EC₅₀ values for dose-dependent binding to human native MUC1 were determined by fitting the dose-response data to the four-parameter logistic model with GraphPad Prism. As shown in FIG. 11 and Table 17, the humanized BG219P antibodies huBG219P-Bz0, E39 and E43 retained comparable binding affinity to native MUC1 compared with chimeric BG219P.

TABLE 17
Comparison of chBG219P and huBG219P binding
affinities to native MUC1 by FACS

| Ab # | ZR-75 binding EC₅₀(nM) |
|---|---|
| chBG219P | 12.3 |
| huBG219P-bz0 | 20.9 |
| huBG219P-E39 | 14.0 |
| huBG219P-E43 | 15.7 |

### Example 9. Generation of scFv of the Murine Anti-Human MUC1 mAb BG138P, BG219P, and BG346P

To generate scFv constructs, the heavy and light chain variable regions (VH, VL) were linked via an artificial amino acid linker. For clone BG138P, the VH and VL from BG138P-Hz2 were linked by (GGGGS)4 linker (SEQ ID NO: 83) with a histidine tag (i.e., HHHHHH (SEQ ID NO: 85)) fused on the C-terminus, making a final construct VH-(GGGGS)4-VL-HHHHHH, designated as scFv1-78P. For clone BG219P, the VH and VL of murine BG219P were used to generate VH-(GGGGS)4-VL-HHHHHH, and additional humanization work was based on this format, details of which are disclosed below. For clone BG346P, VH and VL of murine BG346P were initially linked by (GGGGS)3 linker (SEQ ID NO: 84) to create VH-(GGGGS)3-VL-HHHHHH, and additional engineering work based on this construct is disclosed below.

BG138P scFv Humanization and Engineering

To test if there was room to further improve the physical properties of scFv1-78P, forty constructs with an additional single mutation either in VL or VH were tested for their yield, purity, and thermo-stability. For the experiments, scFv1-78P was fused on a C terminus Fc tag creating scFv-Fc (referred as scFv_{wild type}-Fc), and the series of scFv1-78P mutants were also fused on a C terminus Fc tag creating scFv-Fc (referred as scFvₘᵤₜₐₙₜₛ-Fc). Proteins were overexpressed in Expi293™ cell (Thermo Fisher) and purified using MabSelect SuRe™ column (Cytiva). Yield and purity of resultant scFvₘᵤₜₐₙₜₛ-Fc were compared with scFv_{wild type}-Fc. Twenty-one scFvₘᵤₜₐₙₜₛ-Fc showed better yield compared with scFv_{wild type}-Fc. Two scFvₘᵤₜₐₙₜₛ-Fc showed both better yield and purity; however, neither of them showed better thermo-stability compared with scFv_{wild type}-Fc.

BG219P scFv Humanization and Engineering

The VH and VL of murine BG219P were linked to generate an scFv construct as described above. For humanization, the framework of murine VH and VL were compared to a human germline database. The closest human germlines IGHV3-21*01+IGHJ4 and IGKV1-33*01+IGKJ2 were selected for humanization. Six CDRs in murine scFv were grafted directly into the selected human germline without additional mutations in the framework, resulting in construct scFv2-14P. Affinity and physical property characterization are disclosed below.

To remove a potential post-translational deamidation site in the CDR-H2 region of scFv2-14P without affecting the affinity of the scFv, the vulnerable Asp was mutated to Glu to mimic the structure of the CDR-H2 loop of scFv2-14P. The resulting mutant scFv2-57P showed comparable yield from Expi293™ cells and similar affinity to soluble recombinant MUC1 protein and a MUC1+ cell line as compared with scFv2-14P. Data is shown in Table 18.

TABLE 18
Post-translational site removal of scFv2-14P

| | | Yield | ELISA on Muc1 | | Cell binding on | |
|---|---|---|---|---|---|---|
| scFv2-14P | | compared | SEA domain | | Muc1 + HEK293 | |
| version | Mutation | to WT | Top | EC₅₀ | MFI | EC₅₀ |
| scFv2-14P | N.A. | 1 | 1.026 | 0.0319 | 707411 | 8.646 |
| scFv2-57P | H-D61E | 1.5 | 1.049 | 0.03369 | 611161 | 5.582 |

(N.A.: not applicable. No more mutations added.)

BG346P scFv Humanization and Engineering

The VH and VL from murine BG346P were linked via an artificial linker (GGGGS)3 (SEQ ID NO: 84) to generate murine scFv BG346P. Humanization of murine BG346P scFv was carried out using a framework swap strategy. CDR residues of BG346P were grafted into a series of frameworks listed in Table 19 with additional mutations on the framework as listed.

TABLE 19
Framework used in humanization of BG346P scFv

| BG346P | | | Addition | Addition |
|---|---|---|---|---|
| version | VH | VL | mutations VL | mutations VH |
| scFv3- | IGHV1- | IGKV1- | N.A. | N.A. |
| 001 | 69 | 39*01 | | |
| scFv3- | IGHV1- | IGKV2- | N.A. | N.A. |
| 002 | 69 | 28*01 | | |
| scFv3- | IGHV1- | IGKV3- | N.A. | N.A. |
| 003 | 69 | 20*01 | | |
| scFv3- | IGHV1- | IGKV4- | N.A. | N.A. |
| 004 | 69 | 1*01 | | |
| scFv3- | IGHV2- | IGKV1- | N.A. | N.A. |
| 005 | 05 | 39*01 | | |
| scFv3- | IGHV2- | IGKV2- | N.A. | N.A. |
| 006 | 05 | 28*01 | | |
| scFv3- | IGHV2- | IGKV3- | N.A. | N.A. |
| 007 | 05 | 20*01 | | |
| scFv3- | IGHV2- | IGKV4- | N.A. | N.A. |
| 008 | 05 | 1*01 | | |
| scFv3- | IGHV3- | IGKV1- | N.A. | N.A. |
| 009 | 23 | 39*01 | | |
| scFv3- | IGHV3- | IGKV2- | N.A. | N.A. |
| 010 | 23 | 28*01 | | |
| scFv3- | IGHV3- | IGKV3- | N.A. | N.A. |
| 011 | 23 | 20*01 | | |
| scFv3- | IGHV3- | IGKV4- | N.A. | N.A. |
| 012 | 23 | 1*01- | | |
| scFv3- | IGVH4- | IGKV1- | N.A. | N.A. |
| 013 | 39 | 39*01 | | |
| scFv3- | IGVH4- | IGKV2- | N.A. | N.A. |
| 014 | 39 | 28*01 | | |
| scFv3- | IGVH4- | IGKV3- | N.A. | N.A. |
| 015 | 39 | 20*01 | | |
| scFv3- | IGVH4- | IGKV4- | N.A. | N.A. |
| 016 | 39 | 1*01 | | |
| scFv3- | IGHV5- | IGKV1- | N.A. | N.A. |
| 017 | 51 | 39*01 | | |
| scFv3- | IGHV5- | IGKV2- | N.A. | N.A. |
| 018 | 51 | 28*01 | | |
| scFv3- | IGHV5- | IGKV3- | N.A. | N.A. |
| 019 | 51 | 20*01 | | |
| scFv3- | IGHV5- | IGKV4- | N.A. | N.A. |
| 020 | 51 | 1*01 | | |
| scFv3- | IGHV1- | IGKV1- | 39A, 38K, 43S, | 34M, 35N, 50E, |
| 021 | 69 | 39*01 | 48V, 53N, 55A, | 59N, 60N, 67A, |
| | | | 56D, 71Y, 72S | 69L, 71V, 73K, |
| scFv3- | IGHV1- | IGKV2- | 39A, 36Y, 38K, | 34M, 35N, 50E, |
| 022 | 69 | 28*01 | 48V, 54L, 55A, | 59N, 60N, 67A, |
| | | | 56D, 71Y, 72S | 69L, 71V, 73K, |
| scFv3- | IGHV1- | IGKV3- | 43S, 48V, 53N, | 34M, 35N, 50E, |
| 023 | 69 | 20*01 | 54L, 56D, 58V, | 59N, 60N, 67A, |
| | | | 71Y, 72S | 69L, 71V, 73K, |
| scFv3- | IGHV1- | IGKV4- | 43S, 48V, 53N, | 34M, 35N, 50E, |
| 024 | 69 | 1*01 | 54L, 55A, 56D, | 59N, 60N, 67A, |
| | | | 71Y, 72S | 69L, 71V, 73K, |
| scFv3- | IGHV2- | IGKV1- | 39A, 38K, 43S, | 34M, 35N, 37V, |
| 025 | 05 | 39*01 | 48V, 53N, 55A, | 44G, 49G, 50E, |
| | | | 56D, 71Y, 72S | 58N, 59N, 60N, |
| | | | | 61Q, 63F, 67A, |
| | | | | 69L, 71V, 73K, |
| | | | | 76S, 77T, 78A, |
| | | | | 79Y, 82L |
| scFv3- | IGHV2- | IGKV2- | 39A, 36Y, 38K, | 34M, 35N, 37V, |
| 026 | 05 | 28*01 | 48V, 54L, 55A, | 44G, 49G, 50E, |
| | | | 56D, 71Y, 72S | 58N, 59N, 60N, |
| | | | | 61Q, 63F, 67A, |
| | | | | 69L, 71V, 73K, |
| | | | | 76S, 77T, 78A, |
| | | | | 79Y, 82L |
| scFv3- | IGHV2- | IGKV3- | 43S, 48V, 53N, | 34M, 35N, 37V, |
| 027 | 05 | 20*01 | 54L, 56D, 58V, | 44G, 49G, 50E, |
| | | | 71Y, 72S | 58N, 59N, 60N, |
| | | | | 61Q, 63F, 67A, |
| | | | | 69L, 71V, 73K, |
| | | | | 76S, 77T, 78A, |
| | | | | 79Y, 82L |
| scFv3- | IGHV2- | IGKV4- | 43S, 48V, 53N, | 34M, 35N, 37V, |
| 028 | 05 | 1*01 | 54L, 55A, 56D, | 44G, 49G, 50E, |
| | | | 71Y, 72S | 58N, 59N, 60N, |
| | | | | 61Q, 63F, 67A, |
| | | | | 69L, 71V, 73K, |
| | | | | 76S, 77T, 78A, |
| | | | | 79Y, 82L |
| scFv3- | IGHV3- | IGKV1- | 39A, 38K, 43S, | 34M, 35N, 49G, |
| 029 | 23 | 39*01 | 48V, 53N, 55A, | 50E, 58N, 59N, |
| | | | 56D, 71Y, 72S | 60N, 61Q, 63F, |
| | | | | 67A, 69L, 70T, |
| | | | | 71V, 73K, 76S, |
| | | | | 78A, 82L |
| scFv3- | IGHV3- | IGKV2- | 39A, 36Y, 38K, | 34M, 35N, 49G, |
| 030 | 23 | 28*01 | 48V, 54L, 55A, | 50E, 58N, 59N, |
| | | | 56D, 71Y, 72S | 60N, 61Q, 63F, |
| | | | | 67A, 69L, 70T, |
| | | | | 71V, 73K, 76S, |
| | | | | 78A, 82L |
| scFv3- | IGHV3- | IGKV3- | 43S, 48V, 53N, | 34M, 35N, 49G, |
| 031 | 23 | 20*01 | 54L, 56D, 58V, | 50E, 58N, 59N, |
| | | | 71Y, 72S | 60N, 61Q, 63F, |
| | | | | 67A, 69L, 70T, |
| | | | | 71V, 73K, 76S, |
| | | | | 78A, 82L |
| scFv3- | IGHV3- | IGKV4- | 43S, 48V, 53N, | 34M, 35N, 49G, |
| 032 | 23 | 1*01 | 54L, 55A, 56D, | 50E, 58N, 59N, |
| | | | 71Y, 72S | 60N, 61Q, 63F, |
| | | | | 67A, 69L, 70T, |
| | | | | 71V, 73K, 76S, |
| | | | | 78A, 82L |
| scFv3- | IGVH4- | IGKV1- | 39A, 38K, 43S, | 34M, 35N, 37V, |
| 033 | 39 | 39*01 | 48V, 53N, 55A, | 50E, 58N, 59N, |
| | | | 56D, 71Y, 72S | 61Q, 63F, 67A, |
| | | | | 69L, 70T, 73K, |
| | | | | 76S, 77T, 78A, |
| | | | | 79Y |
| scFv3- | IGVH4- | IGKV2- | 39A, 36Y, 38K, | 34M, 35N, 37V, |
| 034 | 39 | 28*01 | 48V, 54L, 55A, | 50E, 58N, 59N, |
| | | | 56D, 71Y, 72S | 61Q, 63F, 67A, |
| | | | | 69L, 70T, 73K, |
| | | | | 76S, 77T, 78A, |
| | | | | 79Y |
| scFv3- | IGVH4- | IGKV3- | 43S, 48V, 53N, | 34M, 35N, 37V, |
| 035 | 39 | 20*01 | 54L, 56D, 58V, | 50E, 58N, 59N, |
| | | | 71Y, 72S | 61Q, 63F, 67A, |
| | | | | 69L, 70T, 73K, |
| | | | | 76S, 77T, 78A, |
| | | | | 79Y |
| scFv3- | IGVH4- | IGKV4- | 43S, 48V, 53N, | 34M, 35N, 37V, |
| 036 | 39 | 1*01 | 54L, 55A, 56D, | 50E, 58N, 59N, |
| | | | 71Y, 72S | 61Q, 63F, 67A, |
| | | | | 69L, 70T, 73K, |
| | | | | 76S, 77T, 78A, |
| | | | | 79Y |
| scFv3- | IGHV5- | IGKV1- | 39A, 38K, 43S, | 34M, 35N, 50E, |
| 037 | 51 | 39*01 | 48V, 53N, 55A, | 58N, 59N, 60N, |
| | | | 56D, 71Y, 72S | 61Q, 67A, 69L, |
| | | | | 70T, 71V, 82L |
| scFv3- | IGHV5- | IGKV2- | 39A, 36Y, 38K, | 34M, 35N, 50E, |
| 038 | 51 | 28*01 | 48V, 54L, 55A, | 58N, 59N, 60N, |
| | | | 56D, 71Y, 72S | 61Q, 67A, 69L, |
| | | | | 70T, 71V, 82L |
| scFv3- | IGHV5- | IGKV3- | 43S, 48V, 53N, | 34M, 35N, 50E, |
| 039 | 51 | 20*01 | 54L, 56D, 58V, | 58N, 59N, 60N, |
| | | | 71Y, 72S | 61Q, 67A, 69L, |
| | | | | 70T, 71V, 82L |
| scFv3- | IGHV5- | IGKV4- | 43S, 48V, 53N, | 34M, 35N, 50E, |
| 040 | 51 | 1*01 | 54L, 55A, 56D, | 58N, 59N, 60N, |
| | | | 71Y, 72S | 61Q, 67A, 69L, |
| | | | | 70T, 71V, 82L |

(N.A.: not applicable. No more mutations added.)

Humanized scFv constructs listed above were overexpressed in Expi293™ cells (Thermo Fisher). To select the stable scFv constructs, the supernatant of Expi293™ cells with scFv protein overexpressed were kept at 60° C. for two hours. The heated scFv proteins were tested for their ability to bind with recombinant MUC1 SEA domain. After the heat stress experiment, the stable scFv scFv3-65P was selected for further engineering. A longer artificial linker (GGGGS)4 (SEQ ID NO: 83) was introduced into scFv3-65P, substituting for the original (GGGGS)3 (SEQ ID NO: 84) linker, which allowed for better scFv expression. The resulting scFv was named scFv3-96P. In the scFv3-96P background, amino acids with potential post-translational modification risk were substituted with amino acids with similar side chains to generate constructs scFv3-98P, scFv3-99P, and scFv3-00P˜scFv3-07P. Additionally, to minimize immunogenicity, murine back mutations introduced in scFv3-65P were mutated back to human amino acids in the context of scFv3-96P, generating constructs\scFv3-08P˜scFv3-19P. The relative affinities of scFv3-98P, scFv3-99P, and scFv3-00P˜scFv3-19P were compared with scFv3-96P, and any beneficial mutations were then selected. Data is presented in Table 20.

TABLE 20
Additional mutations tested in humanized BG346P

| Protein ID | Addition mutations | Relative EC₅₀ to scFv3-96P |
|---|---|---|
| scFv3-054 | H-W33H | 1.00 |
| scFv3-055 | H-W33L | 1498.80 |
| scFv3-056 | H-M34I | N.A. |
| scFv3-057 | H-M34L | 0.71 |
| scFv3-058 | H-D54E | 0.75 |
| scFv3-059 | H-D54S | 1.93 |
| scFv3-060 | H-D54Q | 2.71 |
| scFv3-061 | H-S55A | 5.01 |
| scFv3-062 | K-W92H | 0.88 |
| scFv3-063 | K-W92L | 12062.90 |
| scFv3-064 | H-E1Q | 1.32 |
| scFv3-065 | H-S16A | 1.42 |
| scFv3-066 | H-A67V | 0.85 |
| scFv3-067 | H-L69M | N.A |
| scFv3-068 | H-V71R | 0.80 |
| scFv3-069 | H-K73T | 0.73 |
| scFv3-070 | H-A78V | 0.51 |
| scFv3-071 | K-K38Q | 0.48 |
| scFv3-072 | K-S43A | 0.74 |
| scFv3-073 | K-V48I | 0.62 |
| scFv3-074 | K-S72T | 0.80 |
| scFv3-075 | K-Y71F | 1.76 |

After each individual mutation was tested for its effect on affinity, mutations that were not harmful to scFv affinity were picked and combined. For amino acids with PTM risk, the goal was to remove them as much as possible without affecting affinity. For the murine mutations, the goal was to transfer as many of them back to human amino acids as possible to minimize immunogenicity. In the end, the construct with the least PTM risk and the least number of murine mutations that maintained affinity was picked as the final scFv, which was BG346P-scFv3-20P (or abbreviated as scFv3-20P), as shown in Table 21.

TABLE 21
Combined mutations tested in humanized BG346P

| | | Relative EC₅₀ to |
|---|---|---|
| | | scFv3-96P shown |
| Protein ID | Additional mutations | by ELISA |
| scFv3-20P | H1Q, H16A, H34I, H69M, H78A | 1.00 |
| scFv3-077 | H1Q, H16A, H34L, H69M, H78A | 0.90 |
| scFv3-078 | H1Q, H16A, H34I, H69M, H78V | 1.01 |
| scFv3-079 | H1Q, H16A, H34L, H69M, H78V | 0.91 |
| scFv3-080 | H1E, H16S, H34I, H69L, H78A | 0.97 |
| scFv3-081 | H1E, H16S, H34L, H69L, H78A | 0.94 |
| scFv3-082 | H1E, H16S, H34I, H69L, H78V | 1.03 |
| scFv3-083 | H1E, H16S, H34L, H69L, H78V | 0.93 |
| scFv3-084 | H1E, H16S, H34I, H69M, H78A | 1.02 |
| scFv3-085 | H1E, H16S, H34L, H69M, H78A | 0.93 |
| scFv3-086 | H1E, H16S, H34I, H69M, H78V | 1.02 |

The sequences of the four scFvs are set forth in Table 22.

TABLE 22
Sequences of selected scFv fragments

| Name | SEQ ID NO | Note | Sequence |
|---|---|---|---|
| scFv1- | SEQ ID NO: 4 | HCDR1 | TYTMH |
| 78P | | (Kabat) | |
| | SEQ ID NO: 5 | HCDR2 | YINPGSDYTHYNQKFKD |
| | | (Kabat) | |
| | SEQ ID NO: 6 | HCDR3 | QGNYAAYYFDY |
| | | (Kabat) | |
| | SEQ ID NO: 7 | LCDR1 | RASGNIHNFLV |
| | | (Kabat) | |
| | SEQ ID NO: 56 | LCDR2 | NAKTLTS |
| | | (Kabat) | |
| | SEQ ID NO: 9 | LCDR3 | QNFWNVPFT |
| | | (Kabat) | |
| | SEQ ID NO: 57 | VH AA | QVQLVQSGAEVKKPGASVKVSCKAS |
| | | | GYTFTTYTMHWVRQAPGQRLEWMG |
| | | | YINPGSDYTHYNQKFKDRVTITADTS |
| | | | ASTAYMELSSLRSEDTAVYYCARQGN |
| | | | YAAYYFDYWGQGTLVTVSS |
| | SEQ ID NO: 60 | VL AA | DIQMTQSPSSLSASVGDRVTITCRASG |
| | | | NIHNFLVWYQQKPGKSPKLLIYNAKT |
| | | | LTSGVPSRFSGSGSGTQYTLTISSLQPE |
| | | | DVATYYCQNFWNVPFTFGQGTKLEIK |
| | SEQ ID NO: 70 | VH-VL | QVQLVQSGAEVKKPGASVKVSCKAS |
| | | full | GYTFTTYTMHWVRQAPGQRLEWMG |
| | | length | YINPGSDYTHYNQKFKDRVTITADTS |
| | | AA | ASTAYMELSSLRSEDTAVYYCARQGN |
| | | | YAAYYFDYWGQGTLVTVSSGGGGS |
| | | | GGGGSGGGGSGGGGSDIQMTQSPSS |
| | | | LSASVGDRVTITCRASGNIHNFLVWY |
| | | | QQKPGKSPKLLIYNAKTLTSGVPSRFS |
| | | | GSGSGTQYTLTISSLQPEDVATYYCQN |
| | | | FWNVPFTFGQGTKLEIK |
| scFv2- | SEQ ID NO: 24 | HCDR1 | DYIMS |
| 14P | | (Kabat) | |
| | SEQ ID NO: 25 | HCDR2 | TVSSGGDNTYYPDSVKG |
| | | (Kabat) | |
| | SEQ ID NO: 26 | HCDR3 | YGGLGAMDY |
| | | (Kabat) | |
| | SEQ ID NO: 27 | LCDR1 | RASGNIHNYLT |
| | | (Kabat) | |
| | SEQ ID NO: 65 | LCDR2 | NAKTLAS |
| | | (Kabat) | |
| | SEQ ID NO: 29 | LCDR3 | QHFWSVPYT |
| | | (Kabat) | |
| | SEQ ID NO: 71 | VH AA | EVQLVESGGGLVKPGGSLRLSCAASG |
| | | | FTFSDYIMSWVRQAPGKGLEWVSTVS |
| | | | SGGDNTYYPDSVKGRFTISRDNAKNS |
| | | | LYLQMNSLRAEDTAVYYCARYGGLG |
| | | | AMDYWGQGTLVTVSS |
| | SEQ ID NO: 72 | VL AA | DIQMTQSPSSLSASVGDRVTITCRASG |
| | | | NIHNYLTWYQQKPGKVPKLLIYNAKT |
| | | | LASGVPSRFSGSGSGTDFTLTISSLQPE |
| | | | DVATYYCQHFWSVPYTFGQGTRLEIK |
| | SEQ ID NO: 73 | VH-VL | EVQLVESGGGLVKPGGSLRLSCAASG |
| | | full | FTFSDYIMSWVRQAPGKGLEWVSTVS |
| | | length | SGGDNTYYPDSVKGRFTISRDNAKNS |
| | | AA | LYLQMNSLRAEDTAVYYCARYGGLG |
| | | | AMDYWGQGTLVTVSSGGGGSGGGGS |
| | | | GGGGSGGGGSDIQMTQSPSSLSASVG |
| | | | DRVTITCRASGNIHNYLTWYQQKPGK |
| | | | VPKLLIYNAKTLASGVPSRFSGSGSGT |
| | | | DFTLTISSLQPEDVATYYCQHFWSVPY |
| | | | TFGQGTRLEIK |
| scFv2- | SEQ ID NO: 24 | HCDR1 | DYIMS |
| 57P | | (Kabat) | |
| | SEQ ID NO: 74 | HCDR2 | TVSSGGDNTYYPESVKG |
| | | (Kabat) | |
| | SEQ ID NO: 26 | HCDR3 | YGGLGAMDY |
| | | (Kabat) | |
| | SEQ ID NO: 27 | LCDR1 | RASGNIHNYLT |
| | | (Kabat) | |
| | SEQ ID NO: 65 | LCDR2 | NAKTLAS |
| | | (Kabat) | |
| | SEQ ID NO: 29 | LCDR3 | QHFWSVPYT |
| | | (Kabat) | |
| | SEQ ID NO: 75 | VH AA | EVQLVESGGGLVKPGGSLRLSCAASG |
| | | | FTFSDYIMSWVRQAPGKGLEWVSTVS |
| | | | SGGDNTYYPESVKGRFTISRDNAKNS |
| | | | LYLQMNSLRAEDTAVYYCARYGGLG |
| | | | AMDYWGQGTLVTVSS |
| | SEQ ID NO: 76 | VLAA | DIQMTQSPSSLSASVGDRVTITCRASG |
| | | | NIHNYLTWYQQKPGKVPKLLIYNAKT |
| | | | LASGVPSRFSGSGSGTDFTLTISSLQPE |
| | | | DVATYYCQHFWSVPYTFGQGTRLEIK |
| | SEQ ID NO: 77 | VH-VL | EVQLVESGGGLVKPGGSLRLSCAASG |
| | | full | FTFSDYIMSWVRQAPGKGLEWVSTVS |
| | | length | SGGDNTYYPESVKGRFTISRDNAKNS |
| | | AA | LYLQMNSLRAEDTAVYYCARYGGLG |
| | | | AMDYWGQGTLVTVSSGGGGSGGGGS |
| | | | GGGGSGGGGSDIQMTQSPSSLSASVG |
| | | | DRVTITCRASGNIHNYLTWYQQKPGK |
| | | | VPKLLIYNAKTLASGVPSRFSGSGSGT |
| | | | DFTLTISSLQPEDVATYYCQHFWSVPY |
| | | | TFGQGTRLEIK |
| scFv3- | SEQ ID NO: 78 | HCDR1 | NYWIN |
| 20P | | (Kabat) | |
| | SEQ ID NO: 79 | HCDR2 | EIDPSDAYTNNNQKFQG |
| | | (Kabat) | |
| | SEQ ID NO: 16 | HCDR3 | GRRTGFAY |
| | | (Kabat) | |
| | SEQ ID NO: 17 | LCDR1 | RASENIYSNLA |
| | | (Kabat) | |
| | SEQ ID NO: 18 | LCDR2 | AATNLAD |
| | | (Kabat) | |
| | SEQ ID NO: 19 | LCDR3 | QHFWGTPYT |
| | | (Kabat) | |
| | SEQ ID NO: 80 | VH AA | QVQLVQSGAEVKKPGASVKVSCKAS |
| | | | GYTFTNYWINWVRQAPGQGLEWMG |
| | | | EIDPSDAYTNNNQKFQGRVTMTVDTS |
| | | | TSTAYMELSSLRSEDTAVYYCARGRR |
| | | | TGFAYWGQGTLVTVSS |
| | SEQ ID NO: 81 | VL AA | DIQMTQSPSSLSASVGDRVTITCRASE |
| | | | NIYSNLAWYQQKPGKAPKLLIYAATN |
| | | | LADGVPSRFSGSGSGTDYTLTISSLQPE |
| | | | DFATYYCQHFWGTPYTFGQGTKLEIK |
| | SEQ ID NO: 82 | VH-VL | QVQLVQSGAEVKKPGASVKVSCKAS |
| | | full | GYTFTNYWINWVRQAPGQGLEWMG |
| | | length | EIDPSDAYTNNNQKFQGRVTMTVDTS |
| | | AA | TSTAYMELSSLRSEDTAVYYCARGRR |
| | | | TGFAYWGQGTLVTVSSGGGGSGGGG |
| | | | SGGGGSGGGGSDIQMTQSPSSLSASV |
| | | | GDRVTITCRASENIYSNLAWYQQKPG |
| | | | KAPKLLIYAATNLADGVPSRFSGSGSG |
| | | | TDYTLTISSLQPEDFATYYCQHFWGTP |
| | | | YTFGQGTKLEIK |

TABLE 23
Sequences of amino acid linker for scFv

| SEQ ID NO | Note | Sequence |
|---|---|---|
| SEQ ID NO: 83 | AA linker | GGGGSGGGGSGGGGSGGGGS |
| SEQ ID NO: 84 | AA linker | GGGGSGGGGSGGGGS |

Affinity Determination of scFv1-78P, scFv2-14P, scFv2-57P, and scFv3-20P

Binding affinities and kinetics constants of purified scFv fragments were determined by surface plasmon resonance (Biacore 8K) at 25° C. The MUC1-SEA-mIgG2a was coupled onto a CM5 Biocore sensor derivatized via amine coupling with a monoclonal rabbit anti-mouse Fc antibody (Cat No. 29215281). The scFv fragments scFv1-78P, scFv2-14P, scFv2-57P, and scFv3-20P were then flowed through via a rate of 30 μl/min. Association of scFv to MUC1 protein was monitored for 2 minutes and disassociation of scFv fragments in buffer 1×HBS (Cytiva, Cat: BR100669) was monitored for 10 mins.

Ka and Kd were determined by fitting the real-time sensorgram to a 1:1 binding model using Biacore™ Insight Evaluation software. Binding disassociation equilibrium constants (K_{D}) were calculated from kinetics rate as: K_{D}(M)=kd/ka. Binding kinetic parameters for the scFv proteins to MUC1 protein are shown in Table 24.

TABLE 24
Biacore binding affinities of scFv at 25° C.

| Antibody | Ka(Ms−1) | Kd(S−1) | KD(Molar) |
|---|---|---|---|
| scFv1-78P | 1.85e+6 | 2.60e−3 | 1.40e−9 |
| scFv2-14P | 8.15e+5 | 4.25e−4 | 5.22e−10 |
| scFv2-57P | 3.97e+5 | 4.35e−4 | 1.10e−9 |
| scFv3-20P | 3.01e+5 | 2.02e−2 | 6.71e−8 |

### Example 10. Binding Activity of scFv Fragments to Native MUC1

To evaluate the binding ability of anti-MUC1 scFvs to MUC1 on the surface of living cells, HEK293 cells were engineered to overexpress MUC1 full length protein as described above. For the assay, 1×10⁶ HEK293-MUC1 cells were seeded into 96-well plates. To generate scFv dose response curves, serially diluted scFv proteins (4.2 pm to 250 nm) were added to cells and the bound scFv was detected using His-tag antibody ifluor 488 (Cat No. A01800). MFI (median fluorescence intensity) of each cell population was measured using Flow Cytometry Instruments—iQue® 3 (Sartorius) and EC₅₀ and Emax of each scFv were determined using four parameter logistic model. The results are shown in Table 25.

The results indicate that all scFvs maintained the affinity to the MUC1 full-length protein on the cell surface. scFv1-78P, scFv2-14P, and scFv2-57P showed better affinity compared with scFv3-20P.

TABLE 25
FACS and MFI of dose dependent binding
of scFv fragments on native MUC1

| Antibody | EC₅₀ (nM) | MFI |
|---|---|---|
| scFv1-78P | 3.873 | 309523 |
| scFv2-14P | 5.083 | 401851 |
| scFv2-57P | 5.247 | 402602 |
| scFv3-20P | 16.15 | 226489 |

### Example 11. Epitope Mapping of the Humanized scFv Fragments

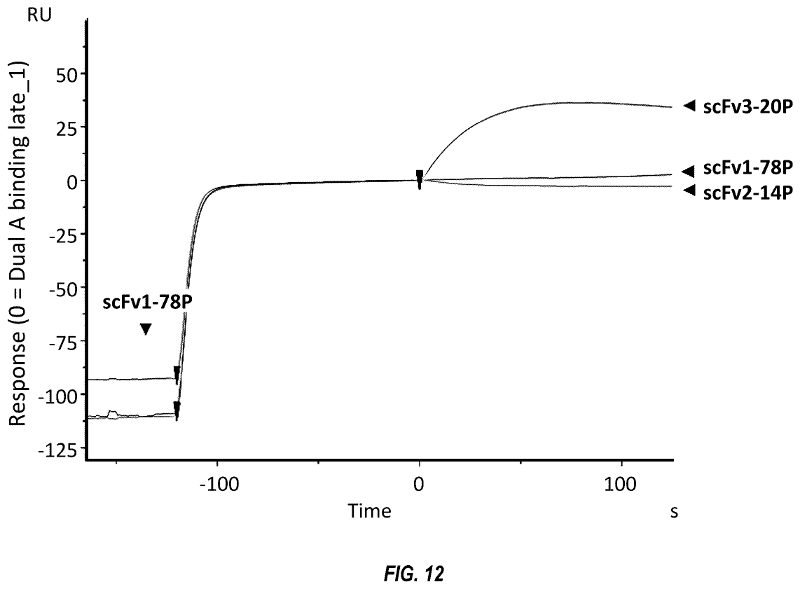

To assess whether the three scFv fragments scFv1-78P, scFv2 (scFv2-14P and scFv2-57P are collectively referred to as “scFv2” in this examples because they have the same epitope bin), and scFv3-20P are able to compete with one another for binding to their respective epitopes on MUC1, a binding competition assay was conducted using the tandem method. Briefly, MUC1-SEA-mIgG2a was immobilized on a CM5 Biocore sensor derivatized via amine coupling with a monoclonal rabbit anti-mouse Fc antibody (Cat No. 29215281). The first scFv fragments were then flowed through at a rate of 10 μl/min. Capture of the first scFv by MUC1-SEA-mIgG2a was monitored for 1.5 min. Then, the second scFv fragments were flowed through at a rate of 10 μl/min, and the capture was monitored for 2 min. Competition between scFv1-78P and scFv2 (scFv2-14P, scFv2-57P) is shown in FIG. 12, while scFv3-20P belongs to another binning.

The results indicate that the epitopes of scFv1-78P, scFv2-14P, and scFv2-57P are at least partially overlapped, while the epitope of scFv3-20P is distinct from the other three.

### Example 12. Physical Properties of the Humanized scFv Fragments

Hydrophobicity, thermostability, and aggregation propensity of humanized scFv fragments were assessed as follows.

### Hydrophobicity Assessment

To determine the hydrophobicity of scFv1-78P, scFv2-14P, scFv2-57P, and scFv3-20P using HIC, 50 μg of sample at 1 mg/ml was diluted with a mobile phase A solution (1.5 M ammonium sulfate, 50 mm sodium phosphate, pH 7.0) to achieve a final ammonium sulfate concentration of about 1M before analysis. A mabpac HIC-10 column from Thermo Fisher was used with a linear gradient of mobile phase A and mobile phase B solution (50 mm sodium phosphate, pH 7.0) over 29 min at a flow rate of 0.5 mg/min. Peak retention times were monitored at A280 absorbance. The results are summarized in Table 26. scFv1-78P (RT, 14.2 min) and scFv3-20P (RT, 9.7 min) showed more hydrophilicity than did scFv2-14P (RT, 20.5 min) and scFv2-57P (RT, 20.6 min). All four scFv domains showed acceptable hydrophobicity.

TABLE 26
Retention time from HIC column

| Sample Name | Retention Time (min) |
|---|---|
| scFv1-78P | 14.2 |
| scFv2-14P | 20.5 |
| scFv2-57P | 20.6 |
| scFv3-20P | 9.7 |

### Thermostability Assessment

The thermal stability of scFv1-78P, scFv2-14P, scFv2-57P, and scFv3-20P, described by the thermal unfolding transition midpoint Tm (° C.) (melting temperature), was measured by extrinsic fluorescence. The Tm was determined using QuantStudio™ 6 Flex System from Applied Biosystems. 20 μl of a 1 mg/ml sample was mixed with 20 μl of 40XSYPRO orange. The plate was scanned from 25° C. to 95° C. at a rate of 0.9° C./min. The Tm was assigned using the first derivative of the raw data from the QuantStudio™ 6 Flex System Analysis software. The results are summarized in Table 27. scFv1-78P (70.0° C.) and scFv3-20P (69.8° C.) showed ˜10° C. higher of Tm than scFv2-14P (60.0° C.) and scFv2-57P (60.2° C.). All four scFv domains showed acceptable thermal stability.

TABLE 27
Tm value from thermal shift assay

| Sample Name | Tm (° C.) |
|---|---|
| scFv1-78P | 70.0 |
| scFv2-14P | 60.0 |
| scFv2-57P | 60.2 |
| scFv3-20P | 69.8 |

### Aggregation Propensity Assessment

To determine the aggregation propensity of scFv1-78P, scFv2-14P, scFv2-57P, and scFv3-20P, static light scattering intensity was measured using an Uncle™ system (Unchained Labs). During the measurement, ˜8.8 μl of protein sample at 1 mg/ml was loaded into a cuvette; the samples were held at 25° C. for 120 s, and then ramped to 95° C. at the rate of 0.3° C./min. The scattering data was collected at an angle of 90° with a laser wavelength of 266 nm. Tagg (aggregation temperature) was analyzed and calculated by Uncle Analysis Software. The results are summarized in Table 28. scFv3-20P (63.8° C.) showed much higher Tagg than did scFv1-78P (56.3° C.), scFv2-14P (54.5° C.), and scFv2-57P (53.1° C.). All four scFv domains showed acceptable Tagg.

TABLE 28
Tagg value from SLS

| Sample Name | Tagg (° C.) |
|---|---|
| scFv1-78P | 56.3 |
| scFv2-14P | 54.5 |
| scFv2-57P | 53.1 |
| scFv3-20P | 63.8 |

1. An anti-MUC1 antibody or antigen-binding fragment thereof that specifically binds to human MUC1, the antibody or antigen-binding fragment thereof comprising:
(i). a heavy chain variable region that comprises (a) a HCDR1 (Heavy Chain Complementarity Determining Region 1) of SEQ ID NO: 4, (b) a HCDR2 of SEQ ID NO: 5, and (c) a HCDR3 of SEQ ID NO: 6, and a light chain variable region that comprises: (d) a LCDR1 of SEQ ID NO: 7, (e) a LCDR2 of SEQ ID NO: 8, and (f) a LCDR3 of SEQ ID NO: 9;
(ii). a heavy chain variable region that comprises (a) a HCDR1 of SEQ ID NO: 4, (b) a HCDR2 of SEQ ID NO: 5, and (c) a HCDR3 of SEQ ID NO: 6, and a light chain variable region that comprises: (d) a LCDR1 of SEQ ID NO: 7, (e) a LCDR2 of SEQ ID NO: 56, and (f) a LCDR3 of SEQ ID NO: 9;
(iii). a heavy chain variable region that comprises (a) a HCDR1 of SEQ ID NO: 14, (b) a HCDR2 of SEQ ID NO: 15, and (c) a HCDR3 of SEQ ID NO: 16, and a light chain variable region that comprises: (d) a LCDR1 of SEQ ID NO: 17, (e) a LCDR2 of SEQ ID NO: 18, and (f) a LCDR3 of SEQ ID NO: 19;
(iv). a heavy chain variable region that comprises (a) a HCDR1 of SEQ ID NO: 78, (b) a HCDR2 of SEQ ID NO: 79, and (c) a HCDR3 of SEQ ID NO: 16, and a light chain variable region that comprises: (d) a LCDR1 of SEQ ID NO: 17, (e) a LCDR2 of SEQ ID NO: 18, and (f) a LCDR3 of SEQ ID NO: 19;
(v). a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (e) a LCDR2 of SEQ ID NO: 28, and (f) a LCDR3 of SEQ ID NO: 29;
(vi). a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (e) a LCDR2 of SEQ ID NO: 65, and (f) a LCDR3 of SEQ ID NO: 29; or
(vii). a heavy chain variable region that comprises (a) a HCDR1 of SEQ ID NO: 24, (b) a HCDR2 of SEQ ID NO: 74, and (c) a HCDR3 of SEQ ID NO: 26, and a light chain variable region that comprises: (d) a LCDR1 of SEQ ID NO: 27, (e) a LCDR2 of SEQ ID NO: 65, and (f) a LCDR3 of SEQ ID NO: 29.

2. The anti-MUC1 antibody or antigen-binding fragment thereof of claim 1, comprising:
(i). a heavy chain variable region (VH) comprising an amino acid sequence at least 90, 91, 92, 93, 94, 95, 96, 97, 98, or 99% identical to SEQ ID NO: 10, and a light chain variable region (VL) comprising an amino acid sequence at least 90, 91, 92, 93, 94, 95, 96, 97, 98, or 99% identical to SEQ ID NO: 11;
(ii). a heavy chain variable region (VH) comprising an amino acid sequence at least 90, 91, 92, 93, 94, 95, 96, 97, 98, or 99% identical to SEQ ID NO: 20, and a light chain variable region (VL) comprising an amino acid sequence at least 90, 91, 92, 93, 94, 95, 96, 97, 98, or 99% identical to SEQ ID NO: 21;
(iii). a heavy chain variable region (VH) comprising an amino acid sequence at least 90, 91, 92, 93, 94, 95, 96, 97, 98, or 99% identical to SEQ ID NO: 30, and a light chain variable region (VL) comprising an amino acid sequence at least 90, 91, 92, 93, 94, 95, 96, 97, 98, or 99% identical to SEQ ID NO: 31;
(iv). a heavy chain variable region (VH) comprising an amino acid sequence at least 90, 91, 92, 93, 94, 95, 96, 97, 98, or 99% identical to SEQ ID NO: 57, and a light chain variable region (VL) comprising an amino acid sequence at least 90, 91, 92, 93, 94, 95, 96, 97, 98, or 99% identical to SEQ ID NO: 59;
(v). a heavy chain variable region (VH) comprising an amino acid sequence at least 90, 91, 92, 93, 94, 95, 96, 97, 98, or 99% identical to SEQ ID NO: 57, and a light chain variable region (VL) comprising an amino acid sequence at least 90, 91, 92, 93, 94, 95, 96, 97, 98, or 99% identical to SEQ ID NO: 60;
(vi). a heavy chain variable region (VH) comprising an amino acid sequence at least 90, 91, 92, 93, 94, 95, 96, 97, 98, or 99% identical to SEQ ID NO: 58, and a light chain variable region (VL) comprising an amino acid sequence at least 90, 91, 92, 93, 94, 95, 96, 97, 98, or 99% identical to SEQ ID NO: 59;
(vii). a heavy chain variable region (VH) comprising an amino acid sequence at least 90, 91, 92, 93, 94, 95, 96, 97, 98, or 99% identical to SEQ ID NO: 58, and a light chain variable region (VL) comprising an amino acid sequence at least 90, 91, 92, 93, 94, 95, 96, 97, 98, or 99% identical to SEQ ID NO: 60;
(viii). a heavy chain variable region (VH) comprising an amino acid sequence at least 90, 91, 92, 93, 94, 95, 96, 97, 98, or 99% identical to SEQ ID NO: 61, and a light chain variable region (VL) comprising an amino acid sequence at least 90, 91, 92, 93, 94, 95, 96, 97, 98, or 99% identical to SEQ ID NO: 62;
(ix). a heavy chain variable region (VH) comprising an amino acid sequence at least 90, 91, 92, 93, 94, 95, 96, 97, 98, or 99% identical to SEQ ID NO: 61, and a light chain variable region (VL) comprising an amino acid sequence at least 90, 91, 92, 93, 94, 95, 96, 97, 98, or 99% identical to SEQ ID NO: 66;
(x). a heavy chain variable region (VH) comprising an amino acid sequence at least 90, 91, 92, 93, 94, 95, 96, 97, 98, or 99% identical to SEQ ID NO: 61, and a light chain variable region (VL) comprising an amino acid sequence at least 90, 91, 92, 93, 94, 95, 96, 97, 98, or 99% identical to SEQ ID NO: 68;
(xi). a heavy chain variable region (VH) comprising an amino acid sequence at least 90, 91, 92, 93, 94, 95, 96, 97, 98, or 99% identical to SEQ ID NO: 71, and a light chain variable region (VL) comprising an amino acid sequence at least 90, 91, 92, 93, 94, 95, 96, 97, 98, or 99% identical to SEQ ID NO: 72;
(xii). a heavy chain variable region (VH) comprising an amino acid sequence at least 90, 91, 92, 93, 94, 95, 96, 97, 98, or 99% identical to SEQ ID NO: 75, and a light chain variable region (VL) comprising an amino acid sequence at least 90, 91, 92, 93, 94, 95, 96, 97, 98, or 99% identical to SEQ ID NO: 76; or
(xiii). a heavy chain variable region (VH) comprising an amino acid sequence at least 90, 91, 92, 93, 94, 95, 96, 97, 98, or 99% identical to SEQ ID NO: 80, and a light chain variable region (VL) comprising an amino acid sequence at least 90, 91, 92, 93, 94, 95, 96, 97, 98, or 99% identical to SEQ ID NO: 81.

3. The anti-MUC1 antibody or antigen-binding fragment thereof of claim 2, comprising:
(i). a heavy chain variable region (VH) that comprises SEQ ID NO: 10, and a light chain variable region (VL) that comprises SEQ ID NO: 11;
(ii). a heavy chain variable region (VH) that comprises SEQ ID NO: 20, and a light chain variable region (VL) that comprises SEQ ID NO: 21;
(iii). a heavy chain variable region (VH) that comprises SEQ ID NO: 30, and a light chain variable region (VL) that comprises SEQ ID NO: 31;
(iv). a heavy chain variable region (VH) that comprises SEQ ID NO: 57, and a light chain variable region (VL) that comprises SEQ ID NO: 59;
(v). a heavy chain variable region (VH) that comprises SEQ ID NO: 57, and a light chain variable region (VL) that comprises SEQ ID NO: 60;
(vi). a heavy chain variable region (VH) that comprises SEQ ID NO: 58, and a light chain variable region (VL) that comprises SEQ ID NO: 59;
(vii). a heavy chain variable region (VH) that comprises SEQ ID NO: 58, and a light chain variable region (VL) that comprises SEQ ID NO: 60;
(viii). a heavy chain variable region (VH) that comprises SEQ ID NO: 61, and a light chain variable region (VL) that comprises SEQ ID NO: 62;
(ix). a heavy chain variable region (VH) that comprises SEQ ID NO: 61, and a light chain variable region (VL) that comprises SEQ ID NO: 66;
(x). a heavy chain variable region (VH) that comprises SEQ ID NO: 61, and a light chain variable region (VL) that comprises SEQ ID NO: 68;
(xi). a heavy chain variable region (VH) that comprises SEQ ID NO: 71, and a light chain variable region (VL) that comprises SEQ ID NO: 72;
(xii). a heavy chain variable region (VH) that comprises SEQ ID NO: 75, and a light chain variable region (VL) that comprises SEQ ID NO: 76; or
(xiii). a heavy chain variable region (VH) that comprises SEQ ID NO: 80, and a light chain variable region (VL) that comprises SEQ ID NO: 81.

4. The anti-MUC1 antibody or antigen-binding fragment thereof of claim 3, wherein one, two, three, four, five, six, seven, eight, nine, or ten amino acids within SEQ ID NO: 10, 11, 20, 21, 30, 31, 57, 58, 59, 60, 61, 62, 66, 68, 71, 72, 75, 76, 80, or 81 have been inserted, deleted, or substituted.

5. The anti-MUC1 antibody or antigen-binding fragment thereof of any one of claims 1 to 4, which is a monoclonal antibody, a chimeric antibody, a humanized antibody, a human engineered antibody, a single chain antibody (scFv), a Fab fragment, a Fab′ fragment, or a F(ab′)2 fragment.

6. The antibody or antigen-binding fragment thereof of claim 5, wherein the antibody or antigen-binding fragment thereof comprises a scFv comprising a VH comprising an amino acid sequence at least 90, 91, 92, 93, 94, 95, 96, 97, 98, or 99% identical to SEQ ID NO: 57, SEQ ID NO: 71, SEQ ID NO: 75, or SEQ ID NO: 80, and a VL comprising an amino acid sequence at least 90, 91, 92, 93, 94, 95, 96, 97, 98, or 99% identical to SEQ ID NO: 60, SEQ ID NO: 72, SEQ ID NO: 76, or SEQ ID NO: 81.

7. The antibody or antigen-binding fragment thereof of claim 6, wherein the antibody or antigen-binding fragment thereof comprises a scFv comprising a VH comprising of SEQ ID NO: 57, SEQ ID NO: 71, SEQ ID NO: 75, or SEQ ID NO: 80, and a VL comprising SEQ ID NO: 60, SEQ ID NO: 72, SEQ ID NO: 76, or SEQ ID NO: 81.

8. The antibody or antigen-binding fragment thereof of claim 7, wherein the antibody or antigen-binding fragment thereof comprises a scFv comprising:
(i). a VH comprising an amino acid sequence at least 90, 91, 92, 93, 94, 95, 96, 97, 98, or 99% identical to SEQ ID NO: 57, and a VL comprising an amino acid sequence at least 90, 91, 92, 93, 94, 95, 96, 97, 98, or 99% identical to SEQ ID NO: 60;
(ii). a VH comprising an amino acid sequence at least 90, 91, 92, 93, 94, 95, 96, 97, 98, or 99% identical to SEQ ID NO: 71, and a VL comprising an amino acid sequence at least 90, 91, 92, 93, 94, 95, 96, 97, 98, or 99% identical to SEQ ID NO: 72;
(iii). a VH comprising an amino acid sequence at least 90, 91, 92, 93, 94, 95, 96, 97, 98, or 99% identical to SEQ ID NO: 75, and a VL comprising an amino acid sequence at least 90, 91, 92, 93, 94, 95, 96, 97, 98, or 99% identical to SEQ ID NO: 76; or
(iv). a VH comprising an amino acid sequence at least 90, 91, 92, 93, 94, 95, 96, 97, 98, or 99% identical to SEQ ID NO: 80, and a VL comprising an amino acid sequence at least 90, 91, 92, 93, 94, 95, 96, 97, 98, or 99% identical to SEQ ID NO: 81.

9. The antibody or antigen-binding fragment thereof of claim 8, wherein the antibody or antigen-binding fragment thereof comprises a scFv comprising:
(i). a VH comprising SEQ ID NO: 57, and a VL comprising SEQ ID NO: 60;
(ii). a VH comprising SEQ ID NO: 71, and a VL comprising SEQ ID NO: 72;
(iii). a VH comprising SEQ ID NO: 75, and a VL comprising SEQ ID NO: 76; or
(iv). a VH comprising SEQ ID NO: 80, and a VL comprising SEQ ID NO: 81.

10. The antibody or antigen-binding fragment thereof of claim 9, wherein one, two, three, four, five, six, seven, eight, nine, or ten amino acids within SEQ ID NO: 57, 60, 71, 72, 75, 76, 80, or 81 have been inserted, deleted or substituted.

11. The antibody or antigen-binding fragment thereof of claim 10, wherein the VH and VL of the scFv are connected via an amino acid linker.

12. The antibody or antigen-binding fragment thereof of claim 11, wherein the amino acid linker comprises SEQ ID NO: 83 or 84.

13. The antibody or antigen-binding fragment thereof of claim 12, wherein the scFv comprises SEQ ID NO: 70, SEQ ID NO: 73, SEQ ID NO: 77, or SEQ ID NO: 82.

14. The anti-MUC1 antibody or antigen-binding fragment thereof of any one of claims 1-13, wherein the antibody or antigen-binding fragment thereof has antibody dependent cellular cytotoxicity (ADCC) or complement dependent cytotoxicity (CDC).

15. The anti-MUC1 antibody or antigen-binding fragment thereof of any one of claims 1-13, wherein the antibody or antigen-binding fragment thereof has reduced glycosylation or no glycosylation or is hypofucosylated.

16. The anti-MUC1 antibody or antigen-binding fragment thereof of any one of claims 1-13, wherein the antibody or antigen-binding fragment thereof comprises increased bisecting GlcNac structures.

17. The anti-MUC1 antibody or antigen-binding fragment thereof of any one of claims 1-13, wherein the Fc domain is an IgG1.

18. A pharmaceutical composition comprising the anti-MUC1 antibody or antigen-binding fragment thereof of any one of claims 1-17, and a pharmaceutically acceptable carrier.

19. An isolated nucleic acid that encodes the anti-MUC1 antibody or antigen-binding fragment thereof of any one of claims 1-17.

20. A vector comprising the nucleic acid of claim 19.

21. A host cell comprising the nucleic acid of claim 19 or the vector of claim 20.

22. A process for producing an anti-MUC1 antibody or antigen-binding fragment thereof, comprising cultivating the host cell of claim 21 and recovering the antibody or antigen-binding fragment from the culture.


SEQUENCE LISTING (85 sequences)

SEQ ID NO: 1 - protein, 1255 aa, Homo sapiens
  REGION 1..1255: note = misc_feature - Human MUC1 (full length)
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 2 - protein, 120 aa, Homo sapiens
  REGION 1..120: note = misc_feature - Human MUC1 SEA domain
LSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAG

SEQ ID NO: 3 - protein, 449 aa, Macaca fascicularis
  REGION 1..449: note = misc_feature - Cyno MUC1 (Full length)
MTPGTQSPFFLLLILTVLTAATVPEPTTVVTGSGHTNSTPGGEKETSATQRSSMPISTKNAVSMTSRLSSHSPVSGSSTTQGQDVTLALAMESATGSATTLGHVVTSAPDTSAAPGSTGPPAHVVTSAPDTSAAPGSTGPPAHVVTSAPDTVTSASDSASGSASTLVHSTTSARATTTPASKSTPFSIPSHHSDTPTTLASHSTKTDASSTHHSTVPPFTSSNHSTSPQLSLGVSFFFLSFHISNLQFNSSLEDPSTNYYQQLQRDISELFLQIYKQGDFLGLSNIMFRPGSVVVQSTLVFREGTTNVHDVETQFNQRKTEAASRYNLTISDISVRDVPFPFSAQTGAGVPGWGIALLVLVCVLVVMAIVYFIALAVCQCRQKNYRQLDIFPARDAYHPMSEYPTYHTHGRYAPAGGTNRSPYEEVSAGNGGSSLSYTNPAVAATSANL

SEQ ID NO: 4 - protein, 5 aa, synthetic construct
  REGION 1..5: note = Synthetic: BG138P, huBG138P-Hz2, scFv1-78P, HCDR1 (Kabat)
TYTMH

SEQ ID NO: 5 - protein, 17 aa, synthetic construct
  REGION 1..17: note = Synthetic: BG138P, huBG138P-Hz2, scFv1-78P, HCDR2 (Kabat)
YINPGSDYTHYNQKFKD

SEQ ID NO: 6 - protein, 11 aa, synthetic construct
  REGION 1..11: note = Synthetic: BG138P, huBG138P-Hz2, scFv1-78P, HCDR3 (Kabat)
QGNYAAYYFDY

SEQ ID NO: 7 - protein, 11 aa, synthetic construct
  REGION 1..11: note = Synthetic: BG138P, huBG138P-Hz2, scFv1-78P, LCDR1 (Kabat)
RASGNIHNFLV

SEQ ID NO: 8 - protein, 7 aa, synthetic construct
  REGION 1..7: note = Synthetic: BG138P, LCDR2 (Kabat)
NAKTLTD

SEQ ID NO: 9 - protein, 9 aa, synthetic construct
  REGION 1..9: note = Synthetic: BG138P, huBG138P-Hz2, scFv1-78P, LCDR3 (Kabat)
QNFWNVPFT

SEQ ID NO: 10 - protein, 120 aa, synthetic construct
  REGION 1..120: note = Synthetic: BG138P, VH AA
QVQLLQSGAELARPGASVKMSCKASGYTFTTYTMHWIKQRPGQGLEWIGYINPGSDYTHYNQKFKDKATLTADKSSSTAYMHLYSLTSEDSAVYYCARQGNYAAYYFDYWGQGTTLTVSS

SEQ ID NO: 11 - protein, 107 aa, synthetic construct
  REGION 1..107: note = Synthetic: BG138P, VL AA
DIQMTQSPASLSASVGETVTITCRASGNIHNFLVWYQQKQGKSPQLLVYNAKTLTDGVPSRFSGSGSGTQYSLKIKSLQPEDFGTYYCQNFWNVPFTFGSGTKLEIK

SEQ ID NO: 12 - DNA, 360 bp, synthetic construct
  misc_feature 1..360: note = Synthetic: BG138P, VH DNA
  source 1..360: mol_type = other DNA
caggtccagttgttacagtctggggctgaactggcaagacctggggcctcagtgaagatgtcctgcaaggcttctggttacacctttactacctacacgatgcactggataaaacagaggcctgggcagggtctggaatggattggatacattaatcctggcagtgattatactcattacaatcagaaattcaaagacaaggccacattgactgcagacaaatcctccagcacagcctacatgcacctgtacagcctgacatctgaggactctgcagtctattactgtgcaagacagggcaactatgccgcgtactactttgactactggggccaaggcaccactctcacagtctcctca

SEQ ID NO: 13 - DNA, 321 bp, synthetic construct
  misc_feature 1..321: note = Synthetic: BG138P, VL DNA
  source 1..321: mol_type = other DNA
gacatccagatgactcagtctccagcctccctatctgcatctgtgggagaaactgtcaccatcacatgtcgagcaagtgggaatattcacaattttttagtatggtatcagcagaaacagggaaaatctcctcagctcctggtctataatgcaaaaaccttaacagatggtgtgccatcaaggttcagtggcagtggatcaggaacacaatattctctcaagatcaagagcctgcagcctgaagattttgggacttattactgtcaaaatttttggaatgttccattcacgttcggctcggggacaaagttggaaataaaa

SEQ ID NO: 14 - protein, 5 aa, synthetic construct
  REGION 1..5: note = Synthetic: BG346P, HCDR1 (Kabat)
NYWMN

SEQ ID NO: 15 - protein, 17 aa, synthetic construct
  REGION 1..17: note = Synthetic: BG346P, HCDR2 (Kabat)
EIDPSDSYTNNNQKFKD

SEQ ID NO: 16 - protein, 8 aa, synthetic construct
  REGION 1..8: note = Synthetic: BG346P, scFv3-20P, HCDR3 (Kabat)
GRRTGFAY

SEQ ID NO: 17 - protein, 11 aa, synthetic construct
  REGION 1..11: note = Synthetic: BG346P, scFv3-20P, LCDR1 (Kabat)
RASENIYSNLA

SEQ ID NO: 18 - protein, 7 aa, synthetic construct
  REGION 1..7: note = Synthetic: BG346P, scFv3-20P, LCDR2 (Kabat)
AATNLAD

SEQ ID NO: 19 - protein, 9 aa, synthetic construct
  REGION 1..9: note = Synthetic: BG346P, scFv3-20P, LCDR3 (Kabat)
QHFWGTPYT

SEQ ID NO: 20 - protein, 117 aa, synthetic construct
  REGION 1..117: note = Synthetic: BG346P, VH AA
EVQLQQSGAELVKPGASVKISCKASGYTFTNYWMNWVRQRPGQGLEWIGEIDPSDSYTNNNQKFKDKATLTVDKSSSTAYMQLSSLTSEDSAVYYCARGRRTGFAYWGQGTLVTVSS

SEQ ID NO: 21 - protein, 107 aa, synthetic construct
  REGION 1..107: note = Synthetic: BG346P, VL AA
DIQMTQSPASLSVSVGETVTITCRASENIYSNLAWYQKKQGKSPQLLVYAATNLADGVPSRFSGSGSGTQYSLKINSLQSEDFGNYYCQHFWGTPYTFGGGTKLEIK

SEQ ID NO: 22 - DNA, 351 bp, synthetic construct
  misc_feature 1..351: note = Synthetic: BG346P, VH DNA
  source 1..351: mol_type = other DNA
gaggtccagcttcagcagtctggggctgagctggtgaagcctggggcttcagtgaagatatcctgcaaggcgtctggctacacgttcaccaactactggatgaactgggtgaggcagaggcctggacaaggccttgagtggatcggagagatcgatccttctgatagttatactaacaacaatcaaaagttcaaggacaaggccacattgactgtagacaaatcctccagcacagcctacatgcagctcagcagcctgacatctgaggactctgcggtctattactgtgcaagagggaggaggacggggtttgcttactggggccaagggactctggtcactgtctcgagc

SEQ ID NO: 23 - DNA, 321 bp, synthetic construct
  misc_feature 1..321: note = Synthetic: BG346P, VL DNA
  source 1..321: mol_type = other DNA
gacatccagatgacccagtctccagcctccctatctgtatctgtgggagaaactgtcaccatcacatgtcgagcaagtgagaatatttacagtaatttagcatggtatcagaagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggaattattactgtcaacatttttggggtactccgtacacgttcggaggggggaccaagctggaaataaaa

SEQ ID NO: 24 - protein, 5 aa, synthetic construct
  REGION 1..5: note = Synthetic: BG219P, huBG219P-Bz0, huBG219P-E39, huBG219P-E43, scFv2-14P, scFv2-57P, HCDR1 (Kabat)
DYIMS

SEQ ID NO: 25 - protein, 17 aa, synthetic construct
  REGION 1..17: note = Synthetic: BG219P, huBG219P-Bz0, huBG219P-E39, huBG219P-E43, scFv2-14P, HCDR2 (Kabat)
TVSSGGDNTYYPDSVKG

SEQ ID NO: 26 - protein, 9 aa, synthetic construct
  REGION 1..9: note = Synthetic: BG219P, huBG219P-Bz0, huBG219P-E39, huBG219P-E43, scFv2-14P, scFv2-57P, HCDR3 (Kabat)
YGGLGAMDY

SEQ ID NO: 27 - protein, 11 aa, synthetic construct
  REGION 1..11: note = Synthetic: BG219P, huBG219P-Bz0, huBG219P-E39, huBG219P-E43, scFv2-14P, scFv2-57P, LCDR1 (Kabat)
RASGNIHNYLT

SEQ ID NO: 28 - protein, 7 aa, synthetic construct
  REGION 1..7: note = Synthetic: BG219P, huBG219P-Bz0, LCDR2 (Kabat)
NAKTLAD

SEQ ID NO: 29 - protein, 9 aa, synthetic construct
  REGION 1..9: note = Synthetic: BG219P, huBG219P-Bz0, huBG219P-E39, huBG219P-E43, scFv2-14P, scFv2-57P, LCDR3 (Kabat)
QHFWSVPYT

SEQ ID NO: 30 - protein, 118 aa, synthetic construct
  REGION 1..118: note = Synthetic: BG219P, VH AA
EVQLVESGGGLVKPGGSLKLSCAASGFTFNDYIMSWVRQTPEKRLEWVATVSSGGDNTYYPDSVKGRFTISRDNAKNNLYLQMSSLRSEDTALYYCTRYGGLGAMDYWGQGTSVTVSS

SEQ ID NO: 31 - protein, 107 aa, synthetic construct
  REGION 1..107: note = Synthetic: BG219P, VL AA
DIVMTQSPASLSASVGETVTITCRASGNIHNYLTWYQQKQGKSPQLLVYNAKTLADGVPSRFSGSGSGPQYSLKINSLQPEDFGSYYCQHFWSVPYTFGGGTRLEIK

SEQ ID NO: 32 - DNA, 350 bp, synthetic construct
  misc_feature 1..350: note = Synthetic: BG219P, VH DNA
  source 1..350: mol_type = other DNA
gaagtgcagctggtggagtctgggggaggcttagtgaagcctggagggtccctgaaactctcctgtgcagcctctggattcactttcaatgactatatcatgtcttgggttcgccagactccggagaagaggctggagtgggtcgcaaccgttagtagtggtggtgataacacctactatccagatagtgtgaagggtcgattcaccatctccagagacaatgccaagaacaacctgtacctgcaaatgagcagtctgaggtctgaggacacggccttgtattactgtacaagatatggggggttaggggctatggactactggggtcaaggaacctcagtcaccgtctc

SEQ ID NO: 33 - DNA, 321 bp, synthetic construct
  misc_feature 1..321: note = Synthetic: BG219P, VL DNA
  source 1..321: mol_type = other DNA
gacattgtgatgacccagtctccagcctccctatctgcatctgtgggagaaactgtcaccatcacatgtcgagcaagtgggaatattcacaattatttaacatggtatcagcagaaacagggaaaatctcctcagctcctggtctataatgcaaaaaccttagcagatggtgtgccatcaaggttcagtggcagtggatcaggaccacaatattctctcaagatcaacagcctgcagcctgaagattttgggagttattactgtcaacatttttggagtgttccgtacacgttcggaggggggaccagactggaaataaaa

SEQ ID NO: 34 - protein, 120 aa, synthetic construct
  REGION 1..120: note = Synthetic: IGHV1-3*01 & JH4
  SITE 31..35: note = Germline sequence with CDRs
  SITE 50..66: note = Germline sequence with CDRs
  SITE 99..109: note = Germline sequence with CDRs
QVQLVQSGAEVKKPGASVKVSCKASGYTFTXXXXXWVRQAPGQRLEWMGXXXXXXXXXXXXXXXXXRVTITRDTSASTAYMELSSLRSEDTAVYYCARXXXXXXXXXXXWGQGTLVTVSS

SEQ ID NO: 35 - protein, 120 aa, synthetic construct
  REGION 1..120: note = Synthetic: BG138P- Hz0_VH, VH (only mouse grafted CDRs?
QVQLVQSGAEVKKPGASVKVSCKASGYTFTTYTMHWVRQAPGQRLEWMGYINPGSDYTHYNQKFKDRVTITRDTSASTAYMELSSLRSEDTAVYYCARQGNYAAYYFDYWGQGTLVTVSS

SEQ ID NO: 36 - protein, 120 aa, synthetic construct
  REGION 1..120: note = Synthetic: BG138P- Bz0_VH (all back mutations), VH (all back mutations)
QVQLVQSGAEVKKPGASVKVSCKASGYTFTTYTMHWIKQAPGQGLEWIGYINPGSDYTHYNQKFKDKATLTADKSASTAYMELYSLRSEDTAVYYCARQGNYAAYYFDYWGQGTLVTVSS

SEQ ID NO: 37 - protein, 120 aa, synthetic construct
  REGION 1..120: note = Synthetic: BG138P- Bz1_VH, VH variant
QVQLVQSGAEVKKPGASVKVSCKASGYTFTTYTMHWVKQAPGQGLEWIGYINPGSDYTHYNQKFKDKATLTADKSASTAYMELYSLRSEDTAVYYCARQGNYAAYYFDYWGQGTLVTVSS

SEQ ID NO: 38 - protein, 120 aa, synthetic construct
  REGION 1..120: note = Synthetic: BG138P- Bz2_VH, VH variant
QVQLVQSGAEVKKPGASVKVSCKASGYTFTTYTMHWIRQAPGQGLEWIGYINPGSDYTHYNQKFKDKATLTADKSASTAYMELYSLRSEDTAVYYCARQGNYAAYYFDYWGQGTLVTVSS

SEQ ID NO: 39 - protein, 120 aa, synthetic construct
  REGION 1..120: note = Synthetic: BG138P- Bz3_VH, VH variant
QVQLVQSGAEVKKPGASVKVSCKASGYTFTTYTMHWIKQAPGQRLEWIGYINPGSDYTHYNQKFKDKATLTADKSASTAYMELYSLRSEDTAVYYCARQGNYAAYYFDYWGQGTLVTVSS

SEQ ID NO: 40 - protein, 120 aa, synthetic construct
  REGION 1..120: note = Synthetic: BG138P- Bz4_VH, VH variant
QVQLVQSGAEVKKPGASVKVSCKASGYTFTTYTMHWIKQAPGQGLEWMGYINPGSDYTHYNQKFKDKATLTADKSASTAYMELYSLRSEDTAVYYCARQGNYAAYYFDYWGQGTLVTVSS

SEQ ID NO: 41 - protein, 120 aa, synthetic construct
  REGION 1..120: note = Synthetic: BG138P- Bz5_VH, VH variant
QVQLVQSGAEVKKPGASVKVSCKASGYTFTTYTMHWIKQAPGQGLEWIGYINPGSDYTHYNQKFKDRATLTADKSASTAYMELYSLRSEDTAVYYCARQGNYAAYYFDYWGQGTLVTVSS

SEQ ID NO: 42 - protein, 120 aa, synthetic construct
  REGION 1..120: note = Synthetic: BG138P- Bz6_VH, VH variant
QVQLVQSGAEVKKPGASVKVSCKASGYTFTTYTMHWIKQAPGQGLEWIGYINPGSDYTHYNQKFKDKVTLTADKSASTAYMELYSLRSEDTAVYYCARQGNYAAYYFDYWGQGTLVTVSS

SEQ ID NO: 43 - protein, 120 aa, synthetic construct
  REGION 1..120: note = Synthetic: BG138P- Bz7_VH, VH variant
QVQLVQSGAEVKKPGASVKVSCKASGYTFTTYTMHWIKQAPGQGLEWIGYINPGSDYTHYNQKFKDKATITADKSASTAYMELYSLRSEDTAVYYCARQGNYAAYYFDYWGQGTLVTVSS

SEQ ID NO: 44 - protein, 120 aa, synthetic construct
  REGION 1..120: note = Synthetic: BG138P- Bz8_VH, VH variant
QVQLVQSGAEVKKPGASVKVSCKASGYTFTTYTMHWIKQAPGQGLEWIGYINPGSDYTHYNQKFKDKATLTRDKSASTAYMELYSLRSEDTAVYYCARQGNYAAYYFDYWGQGTLVTVSS

SEQ ID NO: 45 - protein, 120 aa, synthetic construct
  REGION 1..120: note = Synthetic: BG138P- Bz9_VH, VH variant
QVQLVQSGAEVKKPGASVKVSCKASGYTFTTYTMHWIKQAPGQGLEWIGYINPGSDYTHYNQKFKDKATLTADTSASTAYMELYSLRSEDTAVYYCARQGNYAAYYFDYWGQGTLVTVSS

SEQ ID NO: 46 - protein, 120 aa, synthetic construct
  REGION 1..120: note = Synthetic: BG138P- Bz10_VH, VH variant
QVQLVQSGAEVKKPGASVKVSCKASGYTFTTYTMHWIKQAPGQGLEWIGYINPGSDYTHYNQKFKDKATLTADKSASTAYMELSSLRSEDTAVYYCARQGNYAAYYFDYWGQGTLVTVSS

SEQ ID NO: 47 - protein, 107 aa, synthetic construct
  REGION 1..107: note = Synthetic: Vk1-27*01&JK2
  SITE 24..34: note = Germline sequence with CDRs
  SITE 50..56: note = Germline sequence with CDRs
  SITE 89..97: note = Germline sequence with CDRs
DIQMTQSPSSLSASVGDRVTITCXXXXXXXXXXXWYQQKPGKVPKLLIYXXXXXXXGVPSRFSGSGSGTDFTLTISSLQPEDVATYYCXXXXXXXXXFGQGTKLEIK

SEQ ID NO: 48 - protein, 107 aa, synthetic construct
  REGION 1..107: note = Synthetic: BG138P- Hz0_Vk, Vk variant
DIQMTQSPSSLSASVGDRVTITCRASGNIHNFLVWYQQKPGKVPKLLIYNAKTLTDGVPSRFSGSGSGTDFTLTISSLQPEDVATYYCQNFWNVPFTFGQGTKLEIK

SEQ ID NO: 49 - protein, 107 aa, synthetic construct
  REGION 1..107: note = Synthetic: BG138P- Bz0_Vk, Vk variant
DIQMTQSPSSLSASVGDRVTITCRASGNIHNFLVWYQQKPGKSPKLLVYNAKTLTDGVPSRFSGSGSGTQYSLTISSLQPEDVATYYCQNFWNVPFTFGSGTKLEIK

SEQ ID NO: 50 - protein, 107 aa, synthetic construct
  REGION 1..107: note = Synthetic: BG138P- Bz11_Vk, Vk variant
DIQMTQSPSSLSASVGDRVTITCRASGNIHNFLVWYQQKPGKVPKLLVYNAKTLTDGVPSRFSGSGSGTQYSLTISSLQPEDVATYYCQNFWNVPFTFGSGTKLEIK

SEQ ID NO: 51 - protein, 107 aa, synthetic construct
  REGION 1..107: note = Synthetic: BG138P- Bz12_Vk, Vk variant
DIQMTQSPSSLSASVGDRVTITCRASGNIHNFLVWYQQKPGKSPKLLIYNAKTLTDGVPSRFSGSGSGTQYSLTISSLQPEDVATYYCQNFWNVPFTFGSGTKLEIK

SEQ ID NO: 52 - protein, 107 aa, synthetic construct
  REGION 1..107: note = Synthetic: BG138P- Bz13_Vk, Vk variant
DIQMTQSPSSLSASVGDRVTITCRASGNIHNFLVWYQQKPGKSPKLLVYNAKTLTDGVPSRFSGSGSGTDYSLTISSLQPEDVATYYCQNFWNVPFTFGSGTKLEIK

SEQ ID NO: 53 - protein, 107 aa, synthetic construct
  REGION 1..107: note = Synthetic: BG138P- Bz14_Vk, Vk variant
DIQMTQSPSSLSASVGDRVTITCRASGNIHNFLVWYQQKPGKSPKLLVYNAKTLTDGVPSRFSGSGSGTQFSLTISSLQPEDVATYYCQNFWNVPFTFGSGTKLEIK

SEQ ID NO: 54 - protein, 107 aa, synthetic construct
  REGION 1..107: note = Synthetic: BG138P- Bz15_Vk, Vk variant
DIQMTQSPSSLSASVGDRVTITCRASGNIHNFLVWYQQKPGKSPKLLVYNAKTLTDGVPSRFSGSGSGTQYTLTISSLQPEDVATYYCQNFWNVPFTFGSGTKLEIK

SEQ ID NO: 55 - protein, 107 aa, synthetic construct
  REGION 1..107: note = Synthetic: BG138P- Bz16_Vk, Vk variant
DIQMTQSPSSLSASVGDRVTITCRASGNIHNFLVWYQQKPGKSPKLLVYNAKTLTDGVPSRFSGSGSGTQYSLTISSLQPEDVATYYCQNFWNVPFTFGQGTKLEIK

SEQ ID NO: 56 - protein, 7 aa, synthetic construct
  REGION 1..7: note = Synthetic: huBG138P-Hz2, scFv1-78P, LCDR2 (Kabat)
NAKTLTS

SEQ ID NO: 57 - protein, 120 aa, synthetic construct
  REGION 1..120: note = Synthetic: huBG138P-Hz1 huBG138P-Hz 2, scFv1-78P, VH AA
QVQLVQSGAEVKKPGASVKVSCKASGYTFTTYTMHWVRQAPGQRLEWMGYINPGSDYTHYNQKFKDRVTITADTSASTAYMELSSLRSEDTAVYYCARQGNYAAYYFDYWGQGTLVTVSS

SEQ ID NO: 58 - protein, 120 aa, synthetic construct
  REGION 1..120: note = Synthetic: huBG138P-Hz3 huBG138P-Hz 4, VH AA
QVQLVQSGAEVKKPGASVKVSCKASGYTFTTYTMHWVRQAPGQGLEWMGYINPGSDYTHYNQKFKDRVTITADKSASTAYMELSSLRSEDTAVYYCARQGNYAAYYFDYWGQGTLVTVSS

SEQ ID NO: 59 - protein, 107 aa, synthetic construct
  REGION 1..107: note = Synthetic: huBG138P-Hz1 huBG138P-Hz 3, Vk AA
DIQMTQSPSSLSASVGDRVTITCRASGNIHNFLVWYQQKPGKVPKLLIYNAKTLTSGVPSRFSGSGSGTDYTLTISSLQPEDVATYYCQNFWNVPFTFGQGTKLEIK

SEQ ID NO: 60 - protein, 107 aa, synthetic construct
  REGION 1..107: note = Synthetic: huBG138P-Hz2 huBG138P-Hz 4, scFv1-78P, Vk AA
DIQMTQSPSSLSASVGDRVTITCRASGNIHNFLVWYQQKPGKSPKLLIYNAKTLTSGVPSRFSGSGSGTQYTLTISSLQPEDVATYYCQNFWNVPFTFGQGTKLEIK

SEQ ID NO: 61 - protein, 118 aa, synthetic construct
  REGION 1..118: note = Synthetic: huBG219P-Bz0, huBG219P-E39, huBG219P-E43, VH AA
EVQLLESGGGLVQPGGSLRLSCAASGFTFNDYIMSWVRQAPGKGLEWVATVSSGGDNTYYPDSVKGRFTISRDNSKNTLYLQMNSLRAEDTAVYYCTRYGGLGAMDYWGQGTLVTVSS

SEQ ID NO: 62 - protein, 107 aa, synthetic construct
  REGION 1..107: note = Synthetic: huBG219P-Bz0, VL AA
DIQMTQSPSSLSASVGERVTITCRASGNIHNYLTWYQQKPGKSPKLLVYNAKTLADGVPSRFSGSGSGPDYTLTISSLQPEDFATYYCQHFWSVPYTFGQGTKLEIK

SEQ ID NO: 63 - DNA, 354 bp, synthetic construct
  misc_feature 1..354: note = Synthetic: huBG219P-Bz0, huBG219P-E39, huBG219P-E43, VH DNA
  source 1..354: mol_type = other DNA
gaagtgcagctgctcgagagcgggggcggcctcgtgcaacctggcgggagcctgagactgagctgcgccgctagcggcttcaccttcaacgactacatcatgagctgggtgagacaagcccccggcaagggcctggagtgggtggccaccgtgagcagcgggggcgacaacacctactaccccgacagcgtgaagggcagattcaccatcagcagagacaacagcaagaacaccctgtacctgcagatgaacagcctgagagccgaggacaccgccgtgtactactgcacaagatacggcggcctgggcgccatggactactggggccaaggcaccctggtgaccgtcagctcc

SEQ ID NO: 64 - DNA, 321 bp, synthetic construct
  misc_feature 1..321: note = Synthetic: huBG219P-Bz0, VL DNA
  source 1..321: mol_type = other DNA
gacattcagatgacacagagccctagcagcctgagcgctagcgtgggcgagagagtgaccatcacctgcagagctagcggcaacatccacaactacctgacctggtatcagcagaagcccggcaagagccccaagctgctggtgtacaacgccaagaccctggccgacggcgtgcctagcagattcagcggcagcggcagcggccccgactacaccctgaccatcagcagcctgcagcccgaggacttcgccacctactactgtcagcacttctggagcgtgccctacaccttcggccaaggcaccaagctggagatcaag

SEQ ID NO: 65 - protein, 7 aa, synthetic construct
  REGION 1..7: note = Synthetic: huBG219P-E39, huBG219P-E43, scFv2-14P, scFv2-57P, LCDR2 (Kabat)
NAKTLAS

SEQ ID NO: 66 - protein, 107 aa, synthetic construct
  REGION 1..107: note = Synthetic: huBG219P-E39, VL AA
DIQMTQSPSSLSASVGDRVTITCRASGNIHNYLTWYQQKPGKAPKLLIYNAKTLASGVPSRFSGSGSGTDFTLTISSLQPEDFATYYCQHFWSVPYTFGQGTKLEIK

SEQ ID NO: 67 - DNA, 321 bp, synthetic construct
  misc_feature 1..321: note = Synthetic: huBG219P-E39, VL DNA
  source 1..321: mol_type = other DNA
gacattcagatgacacagagccctagcagcctgagcgctagcgtgggcgacagagtgaccatcacctgcagagctagcggcaacatccacaactacctgacctggtatcagcagaagcccggcaaggcccccaagctgctgatctacaacgccaagaccctggccagcggcgtgcctagcagattcagcggcagcggcagcggcaccgacttcaccctgaccatcagcagcctgcagcccgaggacttcgccacctactactgtcagcacttctggagcgtgccctacaccttcggccaaggcaccaagctggagatcaag

SEQ ID NO: 68 - protein, 107 aa, synthetic construct
  REGION 1..107: note = Synthetic: huBG219P-E43, VL AA
DIQMTQSPSSLSASVGDRVTITCRASGNIHNYLTWYQQKPGKAPKLLIYNAKTLASGVPSRFSGSGSGTDYTLTISSLQPEDFATYYCQHFWSVPYTFGQGTKLEIK

SEQ ID NO: 69 - DNA, 321 bp, synthetic construct
  misc_feature 1..321: note = Synthetic: huBG219P-E43, VL DNA
  source 1..321: mol_type = other DNA
gacattcagatgacacagagccctagcagcctgagcgctagcgtgggcgacagagtgaccatcacctgcagagctagcggcaacatccacaactacctgacctggtatcagcagaagcccggcaaggcccccaagctgctgatctacaacgccaagaccctggccagcggcgtgcctagcagattcagcggcagcggcagcggcaccgactacaccctgaccatcagcagcctgcagcccgaggacttcgccacctactactgtcagcacttctggagcgtgccctacaccttcggccaaggcaccaagctggagatcaag

SEQ ID NO: 70 - protein, 247 aa, synthetic construct
  REGION 1..247: note = Synthetic: scFv1-78P, VH-VL full length AA
QVQLVQSGAEVKKPGASVKVSCKASGYTFTTYTMHWVRQAPGQRLEWMGYINPGSDYTHYNQKFKDRVTITADTSASTAYMELSSLRSEDTAVYYCARQGNYAAYYFDYWGQGTLVTVSSGGGGSGGGGSGGGGSGGGGSDIQMTQSPSSLSASVGDRVTITCRASGNIHNFLVWYQQKPGKSPKLLIYNAKTLTSGVPSRFSGSGSGTQYTLTISSLQPEDVATYYCQNFWNVPFTFGQGTKLEIK

SEQ ID NO: 71 - protein, 118 aa, synthetic construct
  REGION 1..118: note = Synthetic: scFv2-14P, VH AA
EVQLVESGGGLVKPGGSLRLSCAASGFTFSDYIMSWVRQAPGKGLEWVSTVSSGGDNTYYPDSVKGRFTISRDNAKNSLYLQMNSLRAEDTAVYYCARYGGLGAMDYWGQGTLVTVSS

SEQ ID NO: 72 - protein, 107 aa, synthetic construct
  REGION 1..107: note = Synthetic: scFv2-14P, VL AA
DIQMTQSPSSLSASVGDRVTITCRASGNIHNYLTWYQQKPGKVPKLLIYNAKTLASGVPSRFSGSGSGTDFTLTISSLQPEDVATYYCQHFWSVPYTFGQGTRLEIK

SEQ ID NO: 73 - protein, 245 aa, synthetic construct
  REGION 1..245: note = Synthetic: scFv2-14P, VH-VL full length AA
EVQLVESGGGLVKPGGSLRLSCAASGFTFSDYIMSWVRQAPGKGLEWVSTVSSGGDNTYYPDSVKGRFTISRDNAKNSLYLQMNSLRAEDTAVYYCARYGGLGAMDYWGQGTLVTVSSGGGGSGGGGSGGGGSGGGGSDIQMTQSPSSLSASVGDRVTITCRASGNIHNYLTWYQQKPGKVPKLLIYNAKTLASGVPSRFSGSGSGTDFTLTISSLQPEDVATYYCQHFWSVPYTFGQGTRLEIK

SEQ ID NO: 74 - protein, 17 aa, synthetic construct
  REGION 1..17: note = Synthetic: scFv2-57P, HCDR2 (Kabat)
TVSSGGDNTYYPESVKG

SEQ ID NO: 75 - protein, 118 aa, synthetic construct
  REGION 1..118: note = Synthetic: scFv2-57P, VH AA
EVQLVESGGGLVKPGGSLRLSCAASGFTFSDYIMSWVRQAPGKGLEWVSTVSSGGDNTYYPESVKGRFTISRDNAKNSLYLQMNSLRAEDTAVYYCARYGGLGAMDYWGQGTLVTVSS

SEQ ID NO: 76 - protein, 107 aa, synthetic construct; identical to SEQ ID NO: 72
  REGION 1..107: note = Synthetic: scFv2-57P, VL AA

SEQ ID NO: 77 - protein, 245 aa, synthetic construct
  REGION 1..245: note = Synthetic: scFv2-57P, VH-VL full length AA
EVQLVESGGGLVKPGGSLRLSCAASGFTFSDYIMSWVRQAPGKGLEWVSTVSSGGDNTYYPESVKGRFTISRDNAKNSLYLQMNSLRAEDTAVYYCARYGGLGAMDYWGQGTLVTVSSGGGGSGGGGSGGGGSGGGGSDIQMTQSPSSLSASVGDRVTITCRASGNIHNYLTWYQQKPGKVPKLLIYNAKTLASGVPSRFSGSGSGTDFTLTISSLQPEDVATYYCQHFWSVPYTFGQGTRLEIK

SEQ ID NO: 78 - protein, 5 aa, synthetic construct
  REGION 1..5: note = Synthetic: scFv3-20P, HCDR1 (Kabat)
NYWIN

SEQ ID NO: 79 - protein, 17 aa, synthetic construct
  REGION 1..17: note = Synthetic: scFv3-20P, HCDR2 (Kabat)
EIDPSDAYTNNNQKFQG

SEQ ID NO: 80 - protein, 117 aa, synthetic construct
  REGION 1..117: note = Synthetic: scFv3-20P, VH AA
QVQLVQSGAEVKKPGASVKVSCKASGYTFTNYWINWVRQAPGQGLEWMGEIDPSDAYTNNNQKFQGRVTMTVDTSTSTAYMELSSLRSEDTAVYYCARGRRTGFAYWGQGTLVTVSS

SEQ ID NO: 81 - protein, 107 aa, synthetic construct
  REGION 1..107: note = Synthetic: scFv3-20P, VL AA
DIQMTQSPSSLSASVGDRVTITCRASENIYSNLAWYQQKPGKAPKLLIYAATNLADGVPSRFSGSGSGTDYTLTISSLQPEDFATYYCQHFWGTPYTFGQGTKLEIK

SEQ ID NO: 82 - protein, 244 aa, synthetic construct
  REGION 1..244: note = Synthetic: scFv3-20P, VH-VL full length AA
QVQLVQSGAEVKKPGASVKVSCKASGYTFTNYWINWVRQAPGQGLEWMGEIDPSDAYTNNNQKFQGRVTMTVDTSTSTAYMELSSLRSEDTAVYYCARGRRTGFAYWGQGTLVTVSSGGGGSGGGGSGGGGSGGGGSDIQMTQSPSSLSASVGDRVTITCRASENIYSNLAWYQQKPGKAPKLLIYAATNLADGVPSRFSGSGSGTDYTLTISSLQPEDFATYYCQHFWGTPYTFGQGTKLEIK

SEQ ID NO: 83 - protein, 20 aa, synthetic construct
  REGION 1..20: note = Synthetic: AA linker
GGGGSGGGGSGGGGSGGGGS

SEQ ID NO: 84 - protein, 15 aa, synthetic construct
  REGION 1..15: note = Synthetic: AA linker
GGGGSGGGGSGGGGS

SEQ ID NO: 85 - protein, 6 aa, synthetic construct
  REGION 1..6: note = Synthetic:His tag
HHHHHH